(19) United States
(12) Patent Application Publication
(10) Pub. No.: US 2025/0115633 A1
(43) Pub. Date: Apr. 10, 2025

(54) PHOTODYNAMIC THERAPY AND DIAGNOSIS

(71) Applicant: RMW CHO Group Limited, Hong Kong (CN)

(72) Inventors: Sebastian M. Marcuccio, Melbourne (AU); Andrew N. Stephens, Melbourne (AU); Christopher D. Donner, Melbourne (AU); Honsue Cho, Melbourne (AU); Sacha Novakovic, Melbourne (AU); Ramesh Ailuri, Melbourne (AU)

(21) Appl. No.: 18/713,559

(22) PCT Filed: Nov. 28, 2022
(86) PCT No.: PCT/EP2022/083553

§ 371 Date: May 24, 2024

(30) Foreign Application Priority Data
Nov. 26, 2021 (GB) GB2117134.3
Jun. 1, 2022 (GB) GB2208170.7

(51) Int. Cl. C07H 15/26; A61K 31/675; A61K 31/7056; A61K 41/00; A61K 45/06; A61K 49/00; C07D 487/22

(52) U.S. Cl. CPC C07H 15/26; A61K 31/675; A61K 31/7056; A61K 41/0071; A61K 45/06; A61K 49/0036; C07D 487/22

(57) ABSTRACT

The present invention relates to chlorin e4 analogues and their pharmaceutically acceptable salts, and compositions comprising chlorin e4 analogues and their pharmaceutically acceptable salts. Chlorin e4 analogues and pharmaceutically acceptable salts thereof are suitable for use in photodynamic therapy, cytoluminescent therapy and photodynamic diagnosis, for example, for treating or C detecting a tumour, or for antiviral treatment. The present invention also relates to the use of chlorin e4 analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

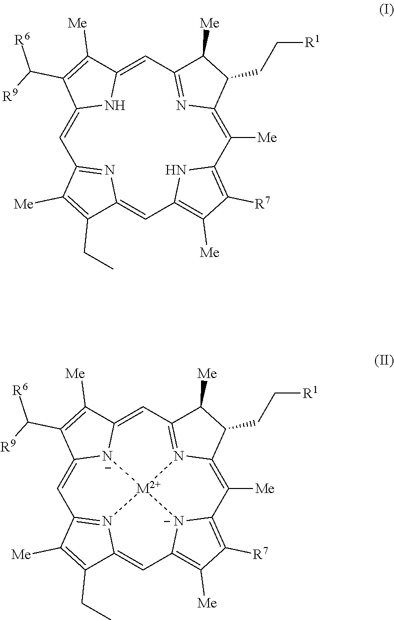

### TECHNICAL FIELD

The present invention relates to chlorin e4 analogues and their pharmaceutically acceptable salts, and compositions comprising chlorin e4 analogues and their pharmaceutically acceptable salts. Chlorin e4 analogues and pharmaceutically acceptable salts thereof are suitable for use in photodynamic therapy, cytoluminescent therapy and photodynamic diagnosis, for example, for treating or detecting a tumour, or for antiviral treatment. The present invention also relates to the use of chlorin e4 analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

The structure of ‘phyllochlorin’ is shown below:

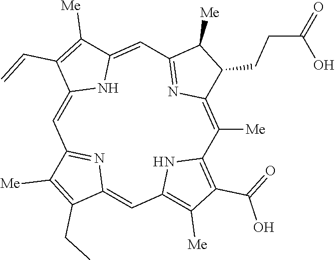 [SMILES: C=Cc1c(C)c2cc3nc(c(C)c4[nH]c(cc5nc(cc1[nH]2)C(C)=C5CC)c(C)c4C(=O)O)[C@@H](CCC(=O)O)[C@@H]3C]

### Chlorin e4 (CAS 550-52-7)

### (7S,8S)-7-(2-carboxyethyl)-18-ethyl-2,5,8,12,17-pentamethyl-13-vinyl-7H,8H-porphyrin-3-carboxylic acid

### BACKGROUND ART

Porphyrins and their analogues are known photosensitive chemical compounds, which can absorb light photons and emit them at higher wavelengths. There are many applications for such unique properties and PDT (photodynamic therapy) is one of them.

Presently, there are two generations of photosensitizers for PDT. The first generation comprises heme porphyrins (blood derivatives), and the second for the most part are chlorophyll analogues. The later compounds are known as chlorins and bacteriochlorins.

Chlorin e4 has been shown to display good photosensitive activity. It was indicated that chlorin e4 has a protective effect against indomethacin-induced gastric lesions in rats and TAA- or CCl4-induced acute liver injuries in mice. It was therefore suggested that chlorin e4 may be a promising new drug candidate for anti-gastrelcosis and liver injury protection. WO 2009/040411 suggests the use of a chlorin e4 zinc complex in photodynamic therapy and WO 2014/091241 suggests the use of chlorin e4 disodium in photodynamic therapy.

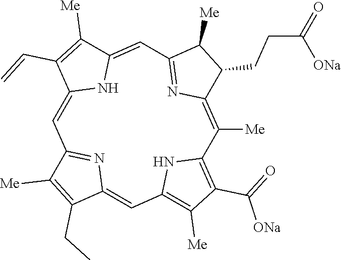

### Chlorin e4 Disodium Salt

However, there is an ongoing need for better photosensitizers. There is a need for compounds that have a high singlet oxygen quantum yield and for compounds that have a strong photosensitizing ability, preferably in organic and aqueous media. There is also a need for compounds that have a high fluorescence quantum yield. In addition, there is a need for compounds and/or compositions which have a higher phototoxicity, a lower dark toxicity, good stability, good solubility, and/or are easily purified.

### SUMMARY OF THE INVENTION

A first aspect of the present invention provides a compound of formula (I) or a complex of formula (II):

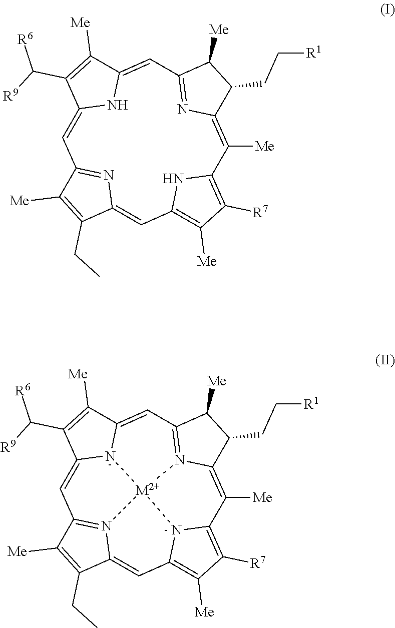

or a pharmaceutically acceptable salt thereof, wherein:
—R¹ is selected from —CH₂OR², —CH₂SR², —CH₂S(O)R², —CH₂S(O)₂R², —CH₂N(R²)₂,
—R², —C(O)—OR³, —C(O)—SR³, —C(O)—N(R³)₂, —C(S)—OR³, —C(S)—SR³ or —C(S)—N(R³)₂; —R², each independently, is selected from —H, —C(O)R⁴, —C(O)—OR⁴, —C(O)—SR⁴, so —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(Rβ)₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}]
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X;
—R⁷ is selected from —C(O)—OR³, —C(O)—SR³, —C(O)—N(R³)₂, —C(S)—OR³, —C(S)—SR³ or —C(S)—N(R³)₂;
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂⁻ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is hydrogen or methyl;
n is 1, 2, 3, 4, 5 or 6;
X is a halo group;
Y is a counter anion;
Z is a counter cation; and
M²⁺ is a metal cation.

The first aspect of the present invention also provides a compound of formula (I) or a complex of formula (II):

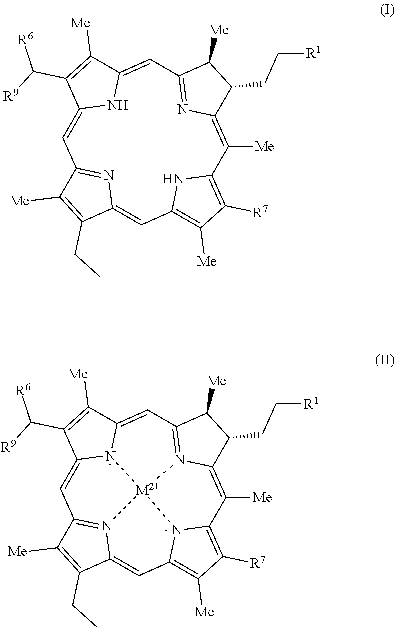

or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]y, —R^{α}—[P(R⁵)₃]Y, —R^{α}—[R⁸]y, —R^{α}—[N(R⁵)₂(R^{5′})], —R^{α}—[P(R⁵)₂(R^{5′})] or —R^{α}—[R^{8′}];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂⁻, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —SR², —S(O)R², —S(O)₂R², or —X;
—R⁷ is selected from —C(O)—OR³, —C(O)—SR³, —C(O)—N(R³)₂, —C(S)—OR³, —C(S)—SR³ or —C(S)—N(R³)₂;
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is hydrogen or methyl;
n is 1, 2, 3, 4, 5 or 6;
X is a halo group;
Y is a counter anion;
Z is a counter cation; and
M²⁺ is a metal cation.

The first aspect of the present invention also provides a compound of formula (I) or a complex of formula (II):

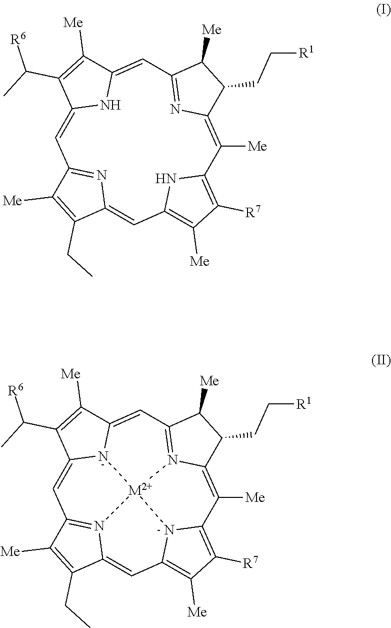

wherein
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁸]Y;
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁸]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁶ is selected from —OR², —SR², —S(O)R², —S(O)₂R², or —X;
—R⁷ is selected from —C(O)—OR³, —C(O)—SR³, —C(O)—N(R³)₂, —C(S)—OR³, —C(S)—SR³ or —C(S)—N(R³)₂;
—R⁸ is —[NC₅H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
Y is a counter ion;
X is a halo group;
M²⁺ is a metal ion;
   or a pharmaceutically acceptable salt thereof.

A second aspect of the present invention provides a compound of formula (I) or a complex of formula (II) according to the first aspect of the invention, for use in medicine.

In one embodiment of the first or second aspect of the present invention, when —R¹ and —R⁷ are both selected from —CO₂H, —CO₂Me or —CONHR³, then —R⁶ is not bromo, —OH, —NH—(C₁-C₁₂ alkyl), —O—(C₁-C₁₂ alkyl), —O—(CH₂)₂—OMe, —O—CO—CF₃, —O-phenyl, —O—CH₂-phenyl, or —O-cyclopentyl.

In the context of the present specification, a “hydrocarbyl” substituent group or a hydrocarbyl moiety in a substituent group only includes carbon and hydrogen atoms but, unless stated otherwise, does not include any heteroatoms, such as N, O, S, P or Se in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O, S, P or Se in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₆₀ hydrocarbyl group, more typically a C₁-C₄, hydrocarbyl group, more typically a C₁-C₂₀ hydrocarbyl group. More typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O, S, P or Se in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O, S, P or Se in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl, thiomorpholinyl, oxetanyl, thietanyl, pyrazolidinyl, imidazolidinyl, dioxolanyl, oxathiolanyl, thianyl and dioxanyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

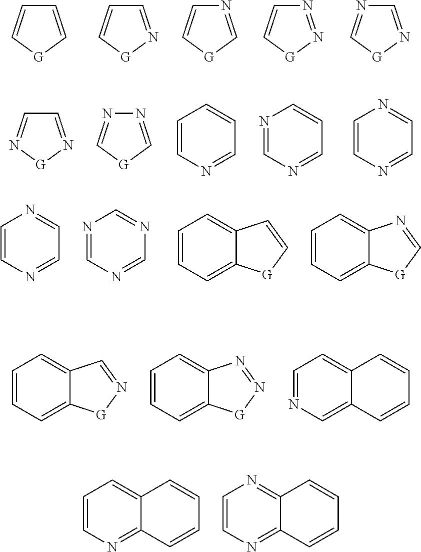

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety (such as —R^{β}):
(i) each hydrogen atom may optionally be replaced by a monovalent substituent independently selected from halo; —CN; —NO₂; —N₃; —R^{x}; —OH; —OR^{x}; —R^{y}-halo; —R^{y}—CN; —R^{y}—NO₂; —R^{y}—N₃; —R^{y}—R^{x}; —R^{y}—OH; —R^{y}—OR^{x}; —SH; —SR^{x}; —SOR^{x}; —SO₂H; —SO₂R^{x}; —SO₂NH₂; —SO₂NHR^{x}; —SO₂N(R^{x})₂; —R^{y}—SH; —R^{y}—SR^{x}; —R^{y}—SOR^{x}; —R^{y}—SO₂H; —R^{y}—SO₂R^{x}; —R^{y}—SO₂NH₂; —R^{y}—SO₂NHR^{x}; —R^{y}—SO₂N(R^{x})₂; —NH₂; —NHR^{x}; —N(R^{x})₂; —N⁺(R^{x})₃; —R^{y}—NH₂; —R^{y}—NHR^{x}; —R^{y}—N(R^{x})₂; —R^{y}—N⁺(R^{x})₃; —CHO; —COR^{x}; —COOH; —COOR^{x}; —OCOR^{x}; —R^{y}—CHO; —R^{y}—COR^{x}; —R^{y}—COOH; —R^{y}—COOR^{x}; or —R^{y}—OCOR^{x}; and/or
(ii) any two hydrogen atoms attached to the same carbon atom may optionally be replaced by a π-bonded substituent independently selected from oxo (═O), ═S, ═NH, or ═NR^{x}; and/or
(iii) any two hydrogen atoms attached to the same or different atoms, within the same optionally substituted group or moiety, may optionally be replaced by a bridging substituent independently selected from —O—, —S—, —NH—, —N(R^{x})—, —N⁺(R^{x})₂— or —R^{y}—;
   wherein each —R^{y}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{x} groups; and
   wherein each —R^{x} is independently selected from a C₁-C₆ alkyl, C₂-C₆ alkenyl, C₂-C₆ alkynyl or C₂-C₆ cyclic group, or wherein any two or three —R^{x} attached to the same nitrogen atom may, together with the nitrogen atom to which they are attached, form a C₂-C₇ cyclic group, and wherein any —R^{x} may optionally be substituted with one or more C₁-C₄ alkyl, C₁-C₄ haloalkyl, —O(C₁-C₄ alkyl), —O(C₁-C₄ haloalkyl), halo, —OH, —NH₂, —CN, or oxo (═O) groups.

Typically a substituted group comprises 1, 2, 3 or 4 substituents, more typically 1, 2 or 3 substituents, more typically 1 or 2 substituents, and more typically 1 substituent.

Unless stated otherwise, any divalent bridging substituent (e.g. —O—, —S—, —NH—, —N(R^{x})—, —N⁺(R^{x})₂— or —R^{y}—) of an optionally substituted group or moiety must only be attached to the specified group or moiety and may not be attached to a second group or moiety, so even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

Where reference is made to a hydrocarbyl or other group including one or more heteroatoms N, O, S, P or Se in its carbon skeleton, or where reference is made to a carbon atom of a hydrocarbyl or other group being replaced by an N, O, S, P or Se atom, what is intended is that:

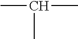

is replaced by

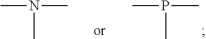

—CH₂— is replaced by —NH—, —PH—, —O—, —S— or —Se—;
—CH₃ is replaced by —NH₂, —PH₂, —OH, —SH or —SeH;
—CH═ is replaced by —N═ or —P═;
CH₂═ is replaced by NH═, PH═, O═, S═ or Se═; or
CH≡ is replaced by N≡ or P≡;
   provided that the resultant group comprises at least one carbon atom. For example, methoxy, dimethylamino and aminoethyl groups are considered to be hydrocarbyl groups including one or more heteroatoms N, O, S, P or Se in their carbon skeleton.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O, S, P or Se, are to be counted as carbon atoms when calculating the number of carbon atoms in a Cₓ-C_{y} group. For example, a morpholinyl group is to be considered a C₆ heterocyclic group, not a C₄ heterocyclic group.

The π electrons of the chlorin ring are delocalised and therefore the chlorin ring can be depicted by more than one resonance structure. Resonance structures are different ways of drawing the same compound. Two of the resonance structures of the chlorin ring are depicted directly below:

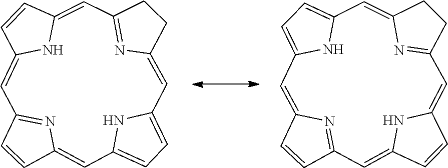 [SMILES: C1=Cc2cc3ccc(cc4nc(cc5ccc(cc1n2)[nH]5)CC4)[nH]3 ; C1=Cc2cc3ccc(cc4nc(cc5ccc(cc1n2)[nH]5)CC4)[nH]3]

Typically a complex comprises a central metal atom or ion known as the coordination centre and a bound molecule or ion which is known as a ligand. In the present specification, the bond between the coordination centre and the ligand is depicted as shown in the complex on the below left (where the attraction between an anionic ligand and a central metal cation is represented by four dashed lines), but equivalently it could be depicted as shown in the complex on the below right (where the attraction between a ligand molecule and a central metal atom is represented by two covalent bonds and two dashed lines):

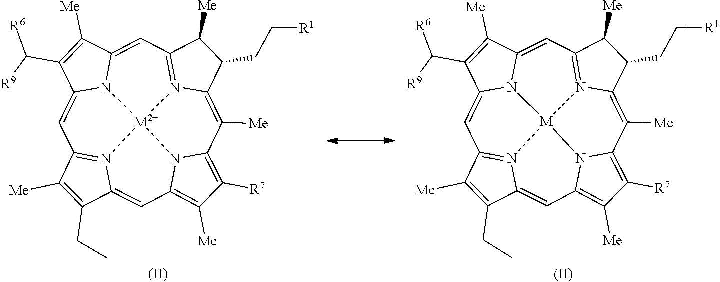

As used herein —[NC₅H₅]Y refers to:

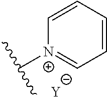

In one embodiment of the first or second aspect of the present invention, X is a halo group selected from fluoro, chloro, bromo, or iodo. In one embodiment, X is chloro or bromo.

In one embodiment of the first or second aspect of the present invention, there is provided a compound of formula (I).

In one embodiment of the first or second aspect of the present invention, Y is a counter anion selected from halides (for example fluoride, chloride, bromide, or iodide) or other inorganic anions (for example bisulfate, hexafluorophosphate (PF6), nitrate, perchlorate, phosphate, or sulfate) or organic anions (for example acetate, ascorbate, aspartate, benzoate, besylate (benzenesulfonate), bicarbonate, bis(trifluoromethanesulfonyl)imide (TFSI), bitartrate, butyrate, camsylate (camphorsulfonate), carbonate, citrate, decanoate, edetate, esylate (ethanesulfonate), fumarate, galactarate, gluceptate, gluconate, glutamate, glycolate, hexanoate, β-hydroxybutyrate, 2-hydroxyethanesulfonate, hydroxymaleate, hydroxynaphthoate, isethionate, lactate, lactobionate, malate, maleate, mandelate, mesylate (methanesulfonate), methylsulfate, mucate, napsylate (naphthalene-2-sulfonate), octanoate, oleate, ornithinate, pamoate, pantothenate, polygalacturonate, propanoate, propionate, salicylate, stearate, succinate, tartrate, teoclate, tetrakis[3,5-bis(trifluoromethyl)phenyl]borate (BARF), tetrakis(pentafluorophenyl)borate (F5-TPB), tetraphenylborate (TPB), tosylate (toluene-p-sulfonate), or triflate (trifluoromethanesulfonate)).

In another embodiment of the first or second aspect of the present invention, Y is a counter anion selected from halides (for example fluoride, chloride, bromide, or iodide) or other inorganic anions (for example bisulfate, nitrate, perchlorate, phosphate, or sulfate) or organic anions (for example acetate, aspartate, benzoate, besylate (benzenesulfonate), butyrate, camsylate (camphorsulfonate), citrate, esylate (ethanesulfonate), fumarate, galactarate, gluconate, glutamate, glycolate, 2-hydroxyethanesulfonate, hydroxymaleate, lactate, malate, maleate, mandelate, mesylate (methanesulfonate), napsylate (naphthalene-2-sulfonate), ornithinate, pamoate, pantothenate, propanoate, salicylate, succinate, tartrate, tosylate (toluene-p-sulfonate), or triflate (trifluoromethanesulfonate)). In one embodiment, Y is fluoride, chloride, bromide or iodide. In one embodiment, Y is chloride or bromide.

In one embodiment of the first or second aspect of the present invention, Z is a counter cation selected from inorganic cations (for example lithium, sodium, potassium, magnesium, calcium or ammonium cation) or organic cations (for example amine cations (for example choline or meglumine cation) or amino acid cations (for example arginine cation).

In one embodiment of the first or second aspect of the present invention, M²⁺ is a metal cation selected from Zn²⁺, Cu²⁺, Fe²⁺, Pd²⁺ or Pt²⁺. In one embodiment, M²⁺ is Zn²⁺.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂. In one embodiment, —R¹ is selected from —C(O)—OR³, —C(O)—SR³, —C(O)—N(R³)₂ or —C(S)—N(R³)₂. In one embodiment, —R¹ is selected from —C(O)—OR³, —C(O)—SR³ or —C(O)—N(R³)₂.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂, and each —R³ is C₁-C₄ alkyl (preferably methyl). In one embodiment, —R¹ is selected from —C(O)—OR³, —C(O)—SR³, —C(O)—N(R³)₂ or —C(S)—N(R³)₂, and each —R³ is C₁-C₄ alkyl (preferably methyl). In one embodiment, —R¹ is selected from —C(O)—OR³, —C(O)—SR³ or —C(O)—N(R³)₂, and each —R³ is C₁-C₄ alkyl (preferably methyl). In one embodiment, —R¹ is —C(O)—OR³ and —R³ is C₁-C₄ alkyl (preferably methyl).

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂, and each —R³ is selected from —R^{α}—OR^{β}, —R^{α}—SR^{β}, —R^{α}—S(O)Rβ or —R^{α}—S(O)₂R^{β}, and —R^{β} is a saccharidyl group. In one embodiment, —R¹ is selected from —C(O)—OR³, —C(O)—SR³ or —C(O)—N(R³)₂, and each —R³ is selected from —R^{α}—OR^{β}, —R^{α}—SR^{β}, —R^{α}—S(O)Rβ or —R^{α}—S(O)₂R^{β}, and —R^{β} is a saccharidyl group. In one embodiment, —R¹ is selected from —C(O)—OR³ or —C(O)—SR³, and —R³ is selected from —R^{α}—ORβ or —R^{α}—SR^{β}, and —R^{β} is a saccharidyl group. Typically in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—OR^{β}, —R^{α}—SR^{β}, —R^{α}—S(O)Rβ or —R^{α}—S(O)₂R^{β}, and —R^{β} is a saccharidyl group, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—OR^{β}, —R^{α}—SR^{β}, —R^{α}—S(O)Rβ or —R^{α}—S(O)₂R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β}, —R^{α}—SR^{β}, —R^{α}—S(O)Rβ or —R^{α}—S(O)₂R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—ORβ or —R^{α}—SR^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β}, —R^{α}—SR^{β}, —R^{α}—S(O)Rβ or —R^{α}—S(O)₂R^{β}, and —R^{β} is a saccharidyl group, and —R^{3′} is C₁-C₄ alkyl (preferably methyl). In one embodiment, —R¹ is —C(O)—N(R³)(R^{3′}), wherein —R³ is selected from —R^{α}—ORβ or —R^{α}—SR^{β}, and —R^{β} is a saccharidyl group, and —R^{3′} is C₁-C₄ alkyl (preferably methyl). Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

An —R^{3′} group refers to an —R³ group attached to the same atom as another —R³ group. —R³ and —R^{3′} may be the same or different. Preferably —R³ and —R^{3′} are different.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—R^{β} or —R^{β}, and —R^{β} is a saccharidyl group, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). Typically in these embodiments, —R^{α}— is a C₁-C₁₂ alkylene group (preferably a C₁-C₈ alkylene group, or a C₁-C₆ alkylene group), a —(CH₂CH₂O)ₘ— group or a —(CH₂CH₂S)ₘ— group, all optionally substituted, wherein m is 1, 2, 3 or 4.

In any of the embodiments in the four preceding paragraphs, the saccharidyl group may optionally be substituted, for example, with a protecting group such as acetyl or a natural amino acid such as valine. Amino acids can be attached to saccharidyl groups, for example, by forming an ester between a carboxylic acid group of the amino acid and a hydroxyl group of the saccharidyl group.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—R^{β} or —R^{β}, and —R^{β} is a C₁-C₈ alkyl group optionally substituted with one or more (such as one, two, three, four, five, six, seven or eight) —OH or —OAc groups, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a C₁-C₈ alkyl group optionally substituted with one or more (such as one, two, three, four, five, six, seven or eight) hydroxyl groups, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a C₁-C₈ alkyl group optionally substituted with one or more (such as one, two, three, four, five, six, seven or eight) hydroxyl groups, and —R^{3′} is H or C₁-C₄ alkyl (preferably methyl). Typically in these embodiments, —R^{α}— is an unsubstituted C₁-C₆ alkylene group, or an unsubstituted C₁-C₄ alkylene group, or an unsubstituted C₁-C₂ alkylene group.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is selected from —R^{α}—H or —R^{α}—OH; —R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is selected from —R^{α}—H or —R^{α}—OH; —R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and —R^{3′} is H or C₁-C₄ alkyl (preferably methyl). In one embodiment, —R¹ is —C(O)—N(R³)(R^{3′}); wherein —R³ is selected from —R^{α}—H or —R^{α}—OH; —R^{α}— is selected from a C₁-C₁₂ alkylene group, wherein one or more carbon atoms in the backbone of the alkylene group may optionally be replaced by one or more heteroatoms O or S; and —R^{3′} is H or C₁-C₄ alkyl (preferably methyl).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{β}; —R^{β} is a C₁-C₁₂ alkyl or C₂-C₁₂ alkenyl group optionally substituted with one or more (such as one, two, three, four or five) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is —R^{β}; —R^{β} is a C₁-C₁₂ alkyl group optionally substituted with one or more (such as one, two, three, four or five) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl). In one embodiment, —R¹ is —C(O)—N(R³)(R^{3′}); wherein —R³ is —R^{β}; —R^{β} is a C₁-C₈ alkyl group optionally substituted with one or more (such as one, two or three) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{Z} is the side chain of a natural amino acid; and —R^{3′} is H or C₁-C₄ alkyl (preferably methyl).

In one embodiment of the first or second aspect of the present invention, —R¹ is selected from —CO—(NR^{zz}—CHR^{z}—CO)ᵥ—N(R^{zz})₂ and —CO—(NR^{zz}—CHR^{z}—CO)ᵥ—OR^{zz}; wherein each —R^{z} is independently selected from the side chains of natural amino acids; each —R^{zz} is independently selected from hydrogen and C₁-C₄ alkyl (preferably methyl); and v is 1, 2, 3, 4, 5, 6, 7 or 8.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{β}; —R^{β} is selected from a C₁-C₂₀ alkyl group, wherein the alkyl group may optionally be substituted with one, two, three or four halo groups, and wherein one, two, three, four, five or six carbon atoms in the backbone of the alkyl group may optionally be replaced by a heteroatom or group independently selected from O, S, NH or NMe; and —R^{3′} is H or C₁-C₄ alkyl (preferably methyl).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); —R^{3′} is H or C₁-C₄ alkyl (preferably methyl); and —R² or —R³ is selected from —R^{α}—[N(R⁵)₃]Y, —R^{α}—[P(R⁵)₃]Y, or —R^{α}—[R⁸]Y. In one embodiment, —R¹ is selected from —R², —C(O)—OR³, —C(O)—SR³, —C(O)—N(R³)(R^{3′}) or —C(S)—N(R³)(R^{3′}); —R^{3′} is H or C₁-C₄ alkyl (preferably methyl); —R² or —R³ is selected from —R^{α}—[N(R⁵)₃]Y, —R^{α}—[P(R⁵)₃]Y, or —R^{α}—[R⁸]Y; each —R⁵ is independently selected from C₁-C₄ alkyl or phenyl wherein the phenyl is optionally substituted with one, two or three C₁-C₄ alkyl or C₁-C₄ alkoxy groups; —R⁸ is —[NC₅H₅] optionally substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). In one embodiment, —R¹ is —C(O)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). Typically in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R¹ is —C(O)—OR³, wherein —R³ is selected from hydrogen, C₁-C₄ alkyl (preferably methyl) or a cation (such as a lithium, sodium, potassium, magnesium, calcium, ammonium, amine (such as choline or meglumine), or amino acid (such as arginine) cation). In one embodiment, —R¹ is —C(O)—OR³, wherein —R³ is selected from C₁-C₄ alkyl (preferably methyl) or a cation (such as a lithium, sodium, potassium, magnesium, calcium, ammonium, amine (such as choline or meglumine), or amino acid (such as arginine) cation).

In one embodiment of the first or second aspect of the present invention, —R¹ is —C(O)—N(R³)₂. In one embodiment, —R¹ is —C(O)—N(C₁-C₄ alkyl)(R³) or —C(O)—NHR³. In one embodiment, —R¹ is —C(O)—N(CH₃)(R³) or —C(O)—NHR³. In one embodiment, —R¹ is —C(O)—N(C₁-C₄ alkyl)(R³). In one embodiment, —R¹ is —C(O)—N(CH₃)(R³).

In one embodiment of the first or second aspect of the present invention, —R¹ is selected from —CH₂OR², —CH₂SR², —CH₂S(O)R², —CH₂S(O)₂R², —CH₂N(R²)₂, or —R². In one embodiment, —R¹ is selected from —CH₂OR², —CH₂SR², —CH₂N(R²)₂, or —R². In one embodiment, —R¹ is selected from —CH₂OR², —CH₂SR², or —CH₂N(R²)₂. In one embodiment, —R¹ is selected from —CH₂OR² or —CH₂SR². In one embodiment, —R¹ is —CH₂OR². In one embodiment, —R¹ is —R², and —R² is —R^{α}—X.

In one embodiment of the first or second aspect of the present invention, —R² is selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, —R² is selected from —R^{α}—OR^{β}, —R^{α}—SR^{β}, —R^{α}—S(O)R^{β} or —R^{α}—S(O)₂R^{β}. In one embodiment, —R² is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R² is selected from —R^{α}—OR^{β} or —R^{α}—SR^{β}. In one embodiment, —R² is selected from —R^{α}—OR^{β} or —R^{α}—SR^{β}, and —R^{β} is a saccharidyl group.

In one embodiment of the first or second aspect of the present invention, —R² is selected from —C(O)R⁴, —C(O)—OR⁴, —C(O)—SR⁴, —C(O)—N(R⁴)₂, —C(S)—OR⁴, —C(S)—SR⁴ or —C(S)—N(R⁴)₂. In one embodiment, —R² is selected from —C(O)R⁴, —C(O)—OR⁴, —C(O)—SR⁴, —C(O)—N(R⁴)₂ or —C(S)—N(R⁴)₂. In one embodiment, —R² is selected from —C(O)R⁴, —C(O)—OR⁴, —C(O)—SR⁴ or —C(O)—N(R⁴)₂.

In one embodiment of the first or second aspect of the present invention, —R² is —C(O)—N(R⁴)(R^{4′}), wherein —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{4′} is H or C₁-C₄ alkyl (preferably methyl). In one embodiment, —R² is —C(O)—N(R⁴)(R^{4′}), wherein —R⁴ is selected from —R^{α}—OR^{β} or —R^{α}—SR^{β}, and —R^{β} is a saccharidyl group, and —R^{4′} is H or C₁-C₄ alkyl (preferably methyl). In one embodiment, —R² is —C(O)—N(R⁴)(R^{4′}), wherein —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{4′} is C₁-C₄ alkyl (preferably methyl). In one embodiment, —R² is —C(O)—N(R⁴)(R^{4′}), wherein —R⁴ is selected from —R^{α}—OR^{β} or —R^{α}—SR^{β}, and —R^{β} is a saccharidyl group, and —R^{4′} is C₁-C₄ alkyl (preferably methyl).

An —R^{4′} group refers to an —R⁴ group attached to the same atom as another —R⁴ group. —R⁴ and —R^{4′} may be the same or different. Preferably —R⁴ and —R^{4′} are different.

In one embodiment of the first or second aspect of the present invention, —R² is —C(O)—N(R⁴)₂. In one embodiment, —R² is —C(O)—N(C₁-C₄ alkyl)(R⁴). In one embodiment, —R² is —C(O)—N(CH₃)(R⁴).

In one embodiment of the first or second aspect of the present invention, —R⁶ is selected from —OR², —N(R²)₂, —SR², —S(O)R² or —S(O)₂R². In one embodiment, —R⁶ is selected from —OR², —SR², —S(O)R² or —S(O)₂R². In one embodiment, —R⁶ is selected from —OR² or —SR². In one embodiment, —R⁶ is —OR².

In one embodiment of the first or second aspect of the present invention, —R⁶ is selected from —OR², —N(R²)₂, —SR², —S(O)R² or —S(O)₂R², and —R² is selected from —H, —C(O)R⁴, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, —R⁶ is selected from —OR², —SR², —S(O)R² or —S(O)₂R², and —R² is selected from —H, —C(O)R⁴, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, —R⁶ is selected from —OR² or —SR², and —R² is selected from —H, —C(O)R⁴, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is selected from —R^{α}—OR^{β}, —R^{α}—SR^{β}, —R^{α}—S(O)R^{β} or —R^{α}—S(O)₂R^{β}; and optionally —R^{β} is a saccharidyl group. In one embodiment, —R⁶ is selected from —OR², —SR², —S(O)R² or —S(O)₂R², and —R² is selected from —R^{α}—OR^{β}, —R^{α}—SR^{β}, —R^{α}—S(O)R^{β} or —R^{α}—S(O)₂R^{β}, and optionally —R^{β} is a saccharidyl group. In one embodiment, —R⁶ is selected from —OR², —SR², —S(O)R² or —S(O)₂R², and —R² is selected from —R^{α}—OR^{β} or —R^{α}—SR^{β}, and optionally —R^{β} is a saccharidyl group. In one embodiment, —R⁶ is selected from —OR² or —SR², and —R² is selected from —R^{α}—OR^{β}, —R^{α}—SR^{β}, —R^{α}—S(O)R^{β} or —R^{α}—S(O)₂R^{β}, and optionally —R^{β} is a saccharidyl group. In one embodiment, —R⁶ is selected from —OR² or —SR², and —R² is selected from —R^{α}—OR^{β} or —R^{α}—SR^{β}, and optionally —R^{β} is a saccharidyl group.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); and —R² is —C(O)R⁴. In one embodiment, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is —C(O)R⁴; —R⁴ is selected from —R^{α}—OR^{β}, —R^{α}—SR^{β}, —R^{α}—S(O)R^{β} or —R^{α}—S(O)₂R^{β}; and —R^{β} is a saccharidyl group. In one embodiment, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is —C(O)R⁴; —R⁴ is selected from —R^{α}—OR^{β} or —R^{α}—SR^{β}; and —R^{β} is a saccharidyl group.

In one embodiment of the first or second aspect of the present invention, —R⁶ is selected from —OR², —SR², —S(O)R² or —S(O)₂R², and —R² is —C(O)R⁴. In one embodiment, —R⁶ is selected from —OR², —SR², —S(O)R² or —S(O)₂R², and —R² is —C(O)R⁴, and —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁶ is selected from —OR², —SR², —S(O)R² or —S(O)₂R², and —R² is —C(O)R⁴, and —R⁴ is selected from —R^{α}—OR^{β} or —R^{α}—SR^{β}, and —R^{β} is a saccharidyl group.

In one embodiment of the first or second aspect of the present invention, —R⁶ is selected from —OR² or —SR², and —R² is —C(O)R⁴. In one embodiment, —R⁶ is selected from —OR² or —SR², and —R² is —C(O)R⁴, and —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁶ is selected from —OR² or —SR², and —R² is —C(O)R⁴, and —R⁴ is selected from —R^{α}—OR^{β} or —R^{α}—SR^{β}, and —R^{β} is a saccharidyl group.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is selected from —R^{β}, —R^{α}—OR^{β}, —R^{α}—SR^{β}, —R^{α}—S(O)R^{β} or —R^{α}—S(O)₂R^{β}; —R^{β} is a saccharidyl group; and —R^{α}— is selected from a C₁-C₁₂ alkylene group, wherein one, two, three or four carbon atoms in the backbone of the alkylene group may optionally be replaced by a heteroatom or group independently selected from O, S, NH or NMe. In one embodiment, —R⁶ is selected from —OR², —N(R²)(R^{2′}) or —SR²; —R^{2′} is selected from hydrogen or C₁-C₄ alkyl (preferably hydrogen or methyl); —R² is selected from —R^{β}, —R^{α}—OR^{β} or —R^{α}—SR^{β}; —R^{β} is a saccharidyl group; and —R^{α}— is selected from a C₁-C₁₂ alkylene group, wherein one, two, three or four carbon atoms in the backbone of the alkylene group may optionally be replaced by a heteroatom or group independently selected from O, S, NH or NMe.

In any of the embodiments in the five preceding paragraphs, the saccharidyl group may optionally be substituted, for example, with a protecting group such as acetyl or a natural amino acid such as valine. Amino acids can be attached to saccharidyl groups, for example, by forming an ester between a carboxylic acid group of the amino acid and a hydroxyl group of the saccharidyl group.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen, C₁-C₄ alkyl or —CO₂(C₁-C₄ alkyl); —R² is selected from —C(O)R⁴, —C(O)—OR⁴, —C(O)—N(R⁴)(R^{4′}), —R^{α}—[N(R⁵)₃]Y, —R^{α}—[P(R⁵)₃]Y, or —R^{α}—[R⁸]Y; —R^{4′} is selected from hydrogen or C₁-C₄ alkyl; and —R⁴ is selected from —R^{α}—[N(R⁵)₃]Y, —R^{c}—[P(R⁵)₃]Y, or —R^{α}—[R⁸]Y. In one embodiment, —R⁶ is selected from —OR², —N(R²)(R^{2′}), —SR², —S(O)R² or —S(O)₂R²; —R^{2′} is selected from hydrogen, C₁-C₄ alkyl or —CO₂(C₁-C₄ alkyl); —R² is selected from —C(O)R⁴, —C(O)—OR⁴, —C(O)—N(R⁴)(R^{4′}), —R^{α}—[N(R⁵)₃]Y, —R^{α}—[P(R⁵)₃]Y, or —R^{α}—[R⁸]Y; —R^{4′} is selected from hydrogen or C₁-C₄ alkyl; —R⁴ is selected from —R^{α}—[N(R⁵)₃]Y, —R^{α}—[P(R⁵)₃]Y, or —R^{α}—[R⁸]Y; each —R⁵ is independently selected from C₁-C₄ alkyl or phenyl wherein the phenyl is optionally substituted with one, two or three C₁-C₄ alkyl or C₁-C₄ alkoxy groups; —R⁸ is —[NC₅H₅] optionally substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide). In one embodiment, —R⁶ is selected from —OR² or —N(R²)(R^{2′}); —R^{2′} is selected from hydrogen, C₁-C₄ alkyl or —CO₂(C₁-C₄ alkyl); —R² is selected from —C(O)R⁴, —C(O)—OR⁴, —C(O)—N(R⁴)(R^{4′}), —R^{α}—[N(R⁵)₃]Y, —R^{α}—[P(R⁵)₃]Y, or —R^{α}—[R⁸]Y; —R^{4′} is selected from hydrogen or C₁-C₄ alkyl; —R⁴ is selected from —R^{α}—[N(R⁵)₃]Y, —R^{α}—[P(R⁵)₃]Y, or —R^{α}—[R⁸]Y; each —R⁵ is independently selected from C₁-C₄ alkyl or phenyl wherein the phenyl is optionally substituted with one, two or three C₁-C₄ alkyl or C₁-C₄ alkoxy groups; —R⁸ is —[NC₅H₅] optionally substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide).

In one embodiment of the first or second aspect of the present invention, —R⁶ is selected from —OR², —N(R²)₂, —SR², —S(O)R² or —S(O)₂R²; and —R² is selected from hydrogen, C₁-C₄ alkyl, —CO(C₁-C₄ alkyl) or —CO₂(C₁-C₄ alkyl). In one embodiment, —R⁶ is selected from —OR² or —N(R²)₂; and —R² is selected from hydrogen, C₁-C₄ alkyl, —CO(C₁-C₄ alkyl) or —CO₂(C₁-C₄ alkyl).

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl; —R² is selected from —R⁴, —C(O)R⁴, —C(O)—OR⁴ or —C(O)—N(R⁴)(R^{4′}); —R^{4′} is selected from hydrogen or C₁-C₄ alkyl; and —R⁴ is selected from a C₁-C₁₂ alkyl group, wherein the alkyl group may optionally be substituted with one, two, three or four halo groups, and wherein one, two, three or four carbon atoms in the backbone of the alkyl group may optionally be replaced by a heteroatom or group independently selected from O, S, NH or NMe. In one embodiment, —R⁶ is selected from —OR² or —N(R²)(R^{2′}); —R^{2′} is selected from hydrogen or C₁-C₄ alkyl; —R² is selected from —R⁴, —C(O)R⁴, —C(O)—OR⁴ or —C(O)—N(R⁴)(R^{4′}); —R^{4′} is selected from hydrogen or C₁-C₄ alkyl; and —R⁴ is selected from a C₁-C₁₂ alkyl group, wherein the alkyl group may optionally be substituted with one, two, three or four halo groups, and wherein one, two, three or four carbon atoms in the backbone of the alkyl group may optionally be replaced by a heteroatom or group independently selected from O, S, NH or NMe.

In the first or second aspect of the present invention, —R⁹ is hydrogen or methyl. In one embodiment, —R⁹ is methyl. In a preferred embodiment, —R⁹ is hydrogen.

In one embodiment of the first or second aspect of the present invention, —R⁷ is selected from —C(O)—OR³, —C(O)—SR³, —C(O)—N(R³)₂, —C(S)—OR³, —C(S)—SR³ or —C(S)—N(R³)₂, and each —R³ is C₁-C₄ alkyl, preferably each —R³ is methyl. In one embodiment, —R⁷ is selected from —C(O)—OR³, —C(O)—SR³ or —C(O)—N(R³)₂, and each —R³ is C₁-C₄ alkyl, preferably each —R³ is methyl. In one embodiment, —R⁷ is —C(O)—OR³, and —R³ is C₁-C₄ alkyl, preferably —R³ is methyl.

In one embodiment of the first or second aspect of the present invention, —R⁷ is —C(O)—OR³, wherein —R³ is selected from hydrogen, C₁-C₄ alkyl (preferably methyl) or a cation (such as a lithium, sodium, potassium, magnesium, calcium, ammonium, amine (such as choline or meglumine), or amino acid (such as arginine) cation).

In one embodiment of the first or second aspect of the present invention, —R⁷ is selected from —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R³ is —R^{β}; —R^{β} is selected from a C₁-C₂₀ alkyl group, wherein the alkyl group may optionally be substituted with one, two, three or four halo groups, and wherein one, two, three, four, five or six carbon atoms in the backbone of the alkyl group may optionally be replaced by a heteroatom or group independently selected from O, S, NH or NMe; and —R^{3′} is H or C₁-C₄ alkyl (preferably methyl).

In one embodiment of the first or second aspect of the present invention, —R⁷ is selected from —C(O)—OR³, —C(O)—SR³, —C(O)—N(R³)₂, —C(S)—OR³, —C(S)—SR³ or —C(S)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁷ is selected from —C(O)—OR³, —C(O)—SR³ or —C(O)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁷ is selected from —C(O)—OR³ or —C(O)—SR³, and —R³ is selected from —R^{α}—OR^{β} or —R^{α}—SR^{β}, and —R^{β} is a saccharidyl group. Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R⁷ is selected from —C(O)—OR³, —C(O)—SR³, —C(O)—N(R³)(R^{3′}) or —C(S)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R⁷ is selected from —C(O)—OR³, —C(O)—SR³ or —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R⁷ is selected from —C(O)—OR³, —C(O)—SR³ or —C(O)—N(R³)(R^{3′}), wherein —R³ is selected from —R^{α}—OR^{β} or —R^{α}—SR^{β}, and —R^{β} is a saccharidyl group, and —R^{3′} is H or C₁-C₄ alkyl (preferably methyl). Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

An —R^{3′} group refers to an —R³ group attached to the same atom as another —R³ group. —R³ and —R^{3′} may be the same or different. Preferably —R³ and —R^{3′} are different.

In one embodiment of the first or second aspect of the present invention, —R⁷ is —C(O)—N(R³)₂. In one embodiment, —R⁷ is —C(O)—N(C₁-C₄ alkyl)(R³) or —C(O)—NHR³. In one embodiment, —R⁷ is —C(O)—N(CH₃)(R³) or —C(O)—NHR³.

In one embodiment of the first or second aspect of the present invention, each —R^{α}— is independently a C₁-C₁₂ alkylene group, a —(CH₂CH₂O)ₘ— group, a —(CH₂CH₂S)ₘ— group, a —(CH₂CH₂O)ₘ—CH₂CH₂— group or a —(CH₂CH₂S)ₘ—CH₂CH₂— group, all optionally substituted, wherein m is 1, 2, 3 or 4. In one embodiment, each —R^{α}— is independently a C₁-C₁₂ alkylene group, a —(CH₂CH₂O)ₘ— group or a —(CH₂CH₂S)ₘ— group, all optionally substituted, wherein m is 1, 2, 3 or 4. In one embodiment, each —R^{α}— is independently a C₁-C₁₂ alkylene group or a —(CH₂CH₂O)ₘ— group, both optionally substituted, wherein m is 1, 2, 3 or 4. In one embodiment, each —R^{α}— is independently an optionally substituted —(CH₂CH₂O)ₘ— group, wherein m is 1, 2, 3 or 4.

In one embodiment of the first or second aspect of the present invention, each —R^{α}— is independently a C₁-C₈ alkylene group, or a C₁-C₆ alkylene group, or a C₂-C₄ alkylene group, all optionally substituted.

In one embodiment of the first or second aspect of the present invention, each —R^{α}— is independently unsubstituted or substituted with one or more substituents independently selected from halo, C₁-C₄ alkyl, or C₁-C₄ haloalkyl. In one embodiment, each —R^{α}— is independently unsubstituted or substituted with one or two substituents independently selected from halo, C₁-C₄ alkyl, or C₁-C₄ haloalkyl. In one embodiment, each —R^{α}— is unsubstituted.

In one embodiment of the first or second aspect of the present invention, each —R^{β} is independently a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In one embodiment of the first or second aspect of the present invention, at least one —R^{β} is independently a C₁-C₆ alkyl group, or a C₁-C₄ alkyl group, or a methyl group, all optionally substituted. In one embodiment, each —R^{β} is independently a C₁-C₆ alkyl group, or a C₁-C₄ alkyl group, or a methyl group, all optionally substituted.

In one embodiment of the first or second aspect of the present invention, at least one —R^{β} is independently a saccharidyl group. In one embodiment, each —R^{β} is independently a saccharidyl group.

In one embodiment of the first or second aspect of the present invention, each —R^{β} is independently unsubstituted or substituted with one or more substituents independently selected from halo, C₁-C₄ alkyl, or C₁-C₄ haloalkyl. In one embodiment, each —R^{β} is independently unsubstituted or substituted with one or two substituents independently selected from halo, C₁-C₄ alkyl, or C₁-C₄ haloalkyl. In one embodiment, each —R^{β} is unsubstituted.

In one embodiment of the first or second aspect of the present invention, each —R³ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, each —R³ is independently selected from —R^{α}—OR^{β}, —R^{α}—SR^{β}, —R^{α}—S(O)R^{β} or —R^{α}—S(O)₂R^{β}. In one embodiment, each —R³ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, each —R³ is independently selected from —R^{α}—OR^{β} or —R^{α}—SR^{β}. In one embodiment, each —R³ is independently selected from —R^{α}—OR^{β} or —R^{α}—SR^{β}, and —R^{β} is a saccharidyl group.

In one embodiment of the first or second aspect of the present invention, each —R⁴ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, each —R⁴ is independently selected from —R^{α}—OR^{β}, —R^{α}—SR^{β}, —R^{α}—S(O)R^{β} or —R^{α}—S(O)₂R^{β}. In one embodiment, each —R⁴ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, each —R⁴ is independently selected from —R^{α}—OR^{β} or —R^{α}—SR^{β}. In one embodiment, each —R⁴ is independently selected from —R^{α}—OR^{β} or —R^{α}—SR^{β}, and —R^{β} is a saccharidyl group.

In one embodiment of the first or second aspect of the present invention, at least one of —R², —R³ or —R⁴ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, at least one of —R², —R³ or —R⁴ is independently selected from —R^{α}—OR^{β} or —R^{α}—SR^{β}, and —R^{β} is a saccharidyl group.

For the purposes of the present invention, a “saccharidyl group” is any group comprising at least one monosaccharide subunit, wherein each monosaccharide subunit may optionally be substituted and/or modified. Typically, a saccharidyl group consist of one or more monosaccharide subunits, wherein each monosaccharide subunit may optionally be substituted and/or modified.

Typically, a carbon atom of a single monosaccharide subunit of each saccharidyl group is directly attached to the remainder of the compound, most typically via a single bond.

For the purposes of the present specification, where it is stated that a first atom or group is “directly attached” to a second atom or group it is to be understood that the first atom or group is covalently bonded to the second atom or group with no intervening atom(s) or group(s) being present. For example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

Typically, each saccharidyl group is derived from the corresponding saccharide by substitution of a hydroxyl group of the saccharide with the group defined by the remainder of the compound.

A single bond between an anomeric carbon of a monosaccharide subunit and a substituent is called a glycosidic bond. A glycosidic group is linked to the anomeric carbon of a monosaccharide subunit by a glycosidic bond. The bond between the saccharidyl group and the remainder of the compound may be a glycosidic or a non-glycosidic bond. Typically, the bond between the saccharidyl group and the remainder of the compound is a glycosidic bond, such that the saccharidyl group is a glycosyl group. Where the bond between the saccharidyl group and the remainder of the compound is a glycosidic bond, the glycosidic bond may be in the a or P configuration. Typically, such a glycosidic bond is in the P configuration.

For the purposes of the present invention, where a saccharidyl group “contains x monosaccharide subunits”, this means that the saccharidyl group has x monosaccharide subunits and no more. In contrast, where a saccharidyl group “comprises x monosaccharide subunits”, this means that the saccharidyl group has x or more monosaccharide subunits.

Each saccharidyl group may be independently selected from a monosaccharidyl, disaccharidyl, oligosaccharidyl or polysaccharidyl group. As will be understood, a monosaccharidyl group contains a single monosaccharide subunit. Similarly, a disaccharidyl group contains two monosaccharide subunits. As used herein, an “oligosaccharidyl group” contains from 2 to 9 monosaccharide subunits. Examples of oligosaccharidyl groups include trisaccharidyl, tetrasaccharidyl, pentasaccharidyl, hexasaccharidyl, heptasaccharidyl, octasaccharidyl and nonasaccharidyl groups. As used herein, a “polysaccharidyl group” contains 10 or more monosaccharide subunits (such as 10-50, or 10-30, or 10-20, or 10-15 monosaccharide subunits).

Each monosaccharide subunit within a disaccharidyl, oligosaccharidyl or polysaccharidyl group may be the same or different. Each monosaccharide subunit within a disaccharidyl, oligosaccharidyl or polysaccharidyl group may be connected to another monosaccharide subunit within the group via a glycosidic or a non-glycosidic bond. Typically each monosaccharide subunit within a disaccharidyl, oligosaccharidyl or polysaccharidyl group is connected to another monosaccharide subunit within the group via a glycosidic bond, which may be in the a or P configuration.

Each oligosaccharidyl or polysaccharidyl group may be a linear, branched or macrocyclic oligosaccharidyl or polysaccharidyl group. Typically, each oligosaccharidyl or polysaccharidyl group is a linear or branched oligosaccharidyl or polysaccharidyl group.

In one embodiment, at least one —R^{β} is a monosaccharidyl or disaccharidyl group.

In a further embodiment, at least one —R^{β} is a monosaccharidyl group. For example, at least one —R^{β} may be a glycosyl group containing a single monosaccharide subunit, wherein the monosaccharide subunit may optionally be substituted and/or modified. Typically at least one —R^{β} is a glycosyl group containing a single monosaccharide subunit, wherein the monosaccharide subunit may optionally be substituted. More typically, at least one —R^{β} is a glycosyl group containing a single monosaccharide subunit, wherein the monosaccharide subunit is unsubstituted.

In one embodiment, at least one —R^{β} is an aldosyl group, wherein the aldosyl group may optionally be substituted and/or modified. For example, at least one —R^{β} may be selected from a glycerosyl, aldotetrosyl (such as erythrosyl or threosyl), aldopentosyl (such as ribosyl, arabinosyl, xylosyl or lyxosyl) or aldohexosyl (such as allosyl, altrosyl, glucosyl, mannosyl, gulosyl, idosyl, galactosyl or talosyl) group, any of which may optionally be substituted and/or modified.

In another embodiment, at least one —R^{β} is a ketosyl group, wherein the ketosyl group may optionally be substituted and/or modified. For example, at least one —R^{β} may be selected from an erythrulosyl, ketopentosyl (such as ribulosyl or xylulosyl) or ketohexosyl (such as psicosyl, fructosyl, sorbosyl or tagatosyl) group, any of which may optionally be substituted and/or modified.

Each monosaccharide subunit may be present in a ring-closed (cyclic) or open-chain (acyclic) form. Typically, each monosaccharide subunit in at least one —R^{β} is present in a ring-closed (cyclic) form. For example, at least one —R^{β} may be a glycosyl group containing a single ring-closed monosaccharide subunit, wherein the monosaccharide subunit may optionally be substituted and/or modified. Typically in such a scenario, at least one —R^{β} is a pyranosyl or furanosyl group, such as an aldopyranosyl, aldofuranosyl, ketopyranosyl or ketofuranosyl group, any of which may optionally be substituted and/or modified. More typically, at least one —R^{β} is a pyranosyl group, such as an aldopyranosyl or ketopyranosyl group, any of which may optionally be substituted and/or modified.

In one embodiment, at least one —R^{β} is selected from a ribopyranosyl, arabinopyranosyl, xylopyranosyl, lyxopyranosyl, allopyranosyl, altropyranosyl, glucopyranosyl, mannopyranosyl, gulopyranosyl, idopyranosyl, galactopyranosyl or talopyranosyl group, any of which may optionally be substituted and/or modified.

In a further embodiment, at least one —R^{β} is a glucosyl group, such as a glucopyranosyl group, wherein the glucosyl or the glucopyranosyl group may optionally be substituted and/or modified. Typically, at least one —R^{β} is a glucosyl group, wherein the glucosyl group is optionally substituted. More typically, at least one —R^{β} is an unsubstituted glucosyl group.

Each monosaccharide subunit may be present in the D- or L-configuration. Typically, each monosaccharide subunit is present in the configuration in which it most commonly occurs in nature.

In one embodiment, at least one —R^{β} is a D-glucosyl group, such as a D-glucopyranosyl group, wherein the D-glucosyl or the D-glucopyranosyl group may optionally be substituted and/or modified. Typically, at least one —R^{β} is a D-glucosyl group, wherein the D-glucosyl group is optionally substituted. More typically, at least one —R^{β} is an unsubstituted D-glucosyl group.

For the purposes of the present invention, in a substituted monosaccharidyl group or monosaccharide subunit:
(a) one or more of the hydroxyl groups of the monosaccharidyl group or monosaccharide subunit are each independently replaced with —H, —F, —Cl, —Br, —I, —CF₃, —CCl₃, —CBr₃, —CI₃, —SH, —NH₂, —N₃, —NH═NH₂, —CN, —NO₂, —COOH, —R^{b}, —O—R^{b}, —S—R^{b}, —R^{a}—O—R^{b}, —R^{a}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{a}—SO—R^{b}, —R^{a}—SO₂—R^{b}, —R^{a}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃+, —R^{a}—N(R^{b})₂, —R^{a}—N(R^{b})₃+, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{a}—CO—N(R^{b})₂; and/or
(b) one, two or three hydrogen atoms directly attached to a carbon atom of the monosaccharidyl group or monosaccharide subunit are each independently replaced with —F, —Cl, —Br, —I, —CF₃, —CCl₃, —CBr₃, —CI₃, —OH, —SH, —NH₂, —N₃, —NH═NH₂, —CN, —NO₂, —COOH, —R^{b}, —O—R^{b}, —S—R^{b}, —R^{a}—O—R^{b}, —R^{a}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{α}—SO—R^{b}, —R^{a}—SO₂—R^{b}, —R^{α}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃+, —R^{a}—N(R^{b})₂, —R^{a}—N(R^{b})₃+, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{a}—CO—N(R^{b})₂; and/or
(c) one or more of the hydroxyl groups of the monosaccharidyl group or monosaccharide subunit, together with the hydrogen attached to the same carbon atom as the hydroxyl group, are each independently replaced with ═O, ═S, ═NR^{b}, or ═N(R^{b})₂⁺; and/or
(d) any two hydroxyl groups of the monosaccharidyl group or monosaccharide subunit are together replaced with —O—R^{c}—, —S—R^{c}—, —SO—R^{c}—, —SO₂—R^{c}—, or —NR^{b}—R^{c}—;
wherein:
   each —R^{a}— is independently a substituted or unsubstituted alkylene, alkenylene or alkynylene group which optionally includes one or more heteroatoms each independently selected from O, N and S in its carbon skeleton and preferably comprises 1-10 carbon atoms;
   each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms; and
   each —R^{c}— is independently a chemical bond, or a substituted or unsubstituted alkylene, alkenylene or alkynylene group which optionally includes one or more heteroatoms each independently selected from O, N and S in its carbon skeleton and preferably comprises 1-10 carbon atoms;
   provided that the monosaccharidyl group or monosaccharide subunit comprises at least one, preferably at least two or at least three, —OH, —O—R^{b}, —O—SO—R^{b}, —O—SO₂—R^{b}, —O—SO₂—OR^{b}, —O—SO—N(R^{b})₂, —O—SO₂—N(R^{b})₂, —OP(R^{b})₂, —OPO(R^{b})₂, —OSi(R^{b})₃, —O—CO—R^{b}, —O—CO—OR^{b}, —O—CO—N(R^{b})₂, or —O—R^{c}—.

Typically, in a substituted monosaccharidyl group or monosaccharide subunit:
(a) one or more of the hydroxyl groups of the monosaccharidyl group or monosaccharide subunit are each independently replaced with —H, —F, —CF₃, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(b) one or two of the hydrogen atoms directly attached to a carbon atom of the monosaccharidyl group or monosaccharide subunit are each independently replaced with —F, —CF₃, —OH, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(c) one hydroxyl group of the monosaccharidyl group or monosaccharide subunit, together with the hydrogen attached to the same carbon atom as the hydroxyl group, is replaced with ═O; and/or
(d) any two hydroxyl groups of the monosaccharidyl group or monosaccharide subunit are together replaced with —O—R^{c}— or —NR^{b}—R^{c}—;
wherein:
   each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms; and
   each —R^{c}— is independently a substituted or unsubstituted alkylene, alkenylene or alkynylene group which optionally includes one, two or three heteroatoms each independently selected from O and N in its carbon skeleton and comprises 1-8 carbon atoms;
   provided that the monosaccharidyl group or monosaccharide subunit comprises at least two, preferably at least three, —OH, —O—R^{b}, —OPO(R^{b})₂, —OSi(R^{b})₃, —O—CO—R^{b}, —O—CO—OR^{b}, —O—CO—N(R^{b})₂, or —O—R^{c}—.

In one embodiment, —R^{β} is a saccharidyl group and one or more of the hydroxyl groups of the saccharidyl group are each independently replaced with —O—CO—R^{b}, wherein each —R^{b} is independently C₁-C₄ alkyl, preferably methyl. In one embodiment, —R^{β} is a saccharidyl group and all of the hydroxyl groups of the saccharidyl group are each independently replaced with —O—CO—R^{b}, wherein each —R^{b} is independently C₁-C₄ alkyl, preferably methyl.

In a modified monosaccharidyl group or monosaccharide subunit:
(a) the ring of the modified monosaccharidyl group or monosaccharide subunit, or what would be the ring in the ring-closed form of the modified monosaccharidyl group or monosaccharide subunit, is partially unsaturated; and/or
(b) the ring oxygen of the modified monosaccharidyl group or monosaccharide subunit, or what would be the ring oxygen in the ring-closed form of the modified monosaccharidyl group or monosaccharide subunit, is replaced with —S— or —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms.

Alternately, where the modified monosaccharide subunit forms part of a disaccharidyl, oligosaccharidyl or polysaccharidyl group, —R^{d} may be a further monosaccharide subunit or subunits forming part of the disaccharidyl, oligosaccharidyl or polysaccharidyl group, wherein any such further monosaccharide subunit or subunits may optionally be substituted and/or modified.

Typically, in a modified monosaccharidyl group or monosaccharide subunit:
(a) the ring of the modified monosaccharidyl group or monosaccharide subunit, or what would be the ring in the ring-closed form of the modified monosaccharidyl group or monosaccharide subunit, contains a single C═C; and/or
(b) the ring oxygen of the modified monosaccharidyl group or monosaccharide subunit, or what would be the ring oxygen in the ring-closed form of the modified monosaccharidyl group or monosaccharide subunit, is replaced with —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms.

Typical examples of substituted and/or modified monosaccharide subunits include those corresponding to:
(i) deoxy sugars, such as deoxyribose, fucose, fuculose and rhamnose, wherein a hydroxyl group of the monosaccharidyl group or monosaccharide subunit has been replaced by —H;
(ii) amino sugars, such as glucosamine and galactosamine, wherein a hydroxyl group of the monosaccharidyl group or monosaccharide subunit has been replaced by —NH₂, most typically at the 2-position; and
(iii) sugar acids, containing a —COOH group, such as aldonic acids (e.g. gluconic acid), ulosonic acids, uronic acids (e.g. glucuronic acid) and aldaric acids (e.g. gularic or galactaric acid).

In one embodiment of the first or second aspect of the present invention, at least one —R^{β} is a monosaccharidyl group selected from:

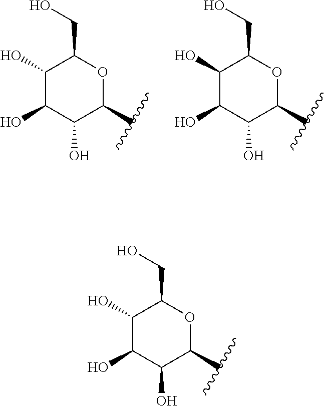 [SMILES: *[C@@H]1O[C@H](CO)[C@@H](O)[C@H](O)[C@H]1O ; *[C@@H]1O[C@H](CO)[C@H](O)[C@H](O)[C@H]1O ; *[C@@H]1O[C@H](CO)[C@@H](O)[C@H](O)[C@@H]1O]

Preferably in the compound or complex according to the first or second aspect of the present invention, at least one —R^{β} is:

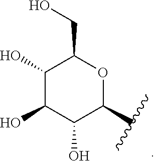 [SMILES: *[C@@H]1O[C@H](CO)[C@@H](O)[C@H](O)[C@H]1O]

In one embodiment of the first or second aspect of the present invention, at least one of —R², —R³ or —R⁴ is independently selected from —R^{α}—OR^{β}, —R^{α}—SR^{β}, —R^{α}—S(O)R^{β} or —R^{α}—S(O)₂R^{β} (preferably from —R^{α}—OR^{β} or —R^{α}—SR^{β}), and —R^{β} is selected from:

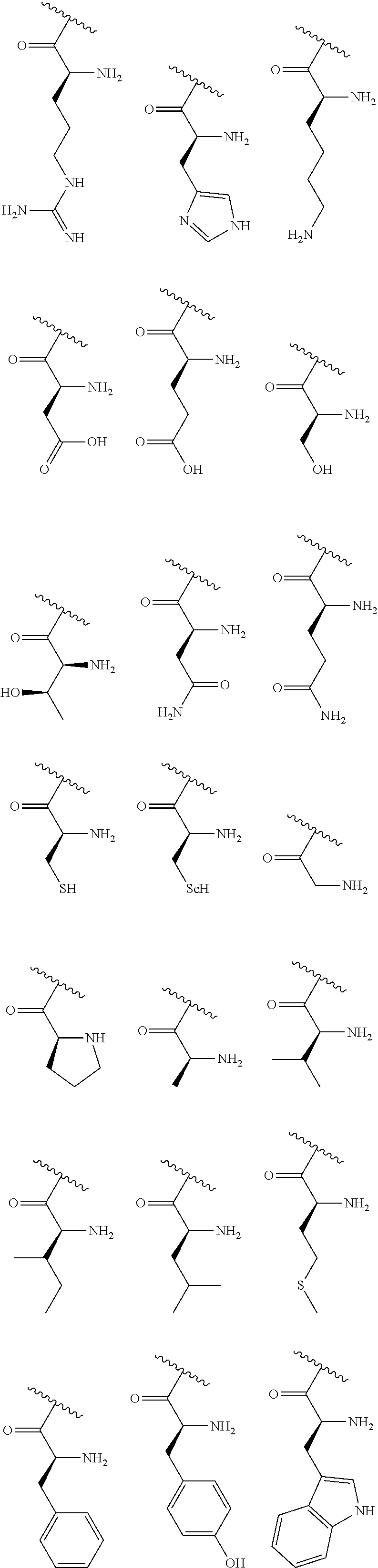

In one embodiment of the first or second aspect of the present invention, at least one of —R², —R³ or —R⁴ is independently selected from —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})], or —R^{α}—[R^{8′}]. In one embodiment, at least one of —R², —R³ or —R⁴ is independently selected from —R^{α}—[N(R⁵)₃]Y, —R^{α}—[P(R⁵)₃]Y, or —R^{α}—[R⁸]Y. In one embodiment, at least one of —R², —R³ or —R⁴ is independently selected from:

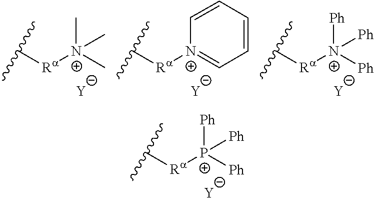

In the first or second aspect of the present invention, each —R⁵ may be the same or different. In a preferred embodiment, each —R⁵ is the same.

In one embodiment of the first or second aspect of the present invention, each —R⁵ is independently unsubstituted or substituted with one or two substituents. In one embodiment, each —R⁵ is unsubstituted.

In one embodiment of the first or second aspect of the present invention, —R⁸ is unsubstituted or substituted with one or two substituents. In one embodiment, —R⁸ is unsubstituted.

In one embodiment, —R⁸ is not substituted at the 4-position of the pyridine ring with a halo group. In one embodiment, —R⁸ is unsubstituted at the 4-position of the pyridine ring. In one embodiment, —R⁸ is unsubstituted.

In one embodiment of the first or second aspect of the present invention, each of —R¹, —R⁶ and —R⁷ independently comprises from 1 to 100 atoms other than hydrogen, preferably from 1 to 80 atoms other than hydrogen, preferably from 1 to 60 atoms other than hydrogen, preferably from 1 to 50 atoms other than hydrogen, and preferably from 1 to 45 atoms other than hydrogen.

In a particularly preferred embodiment, the first or second aspect of the present invention provides a compound of formula (I) or a complex of formula (II):

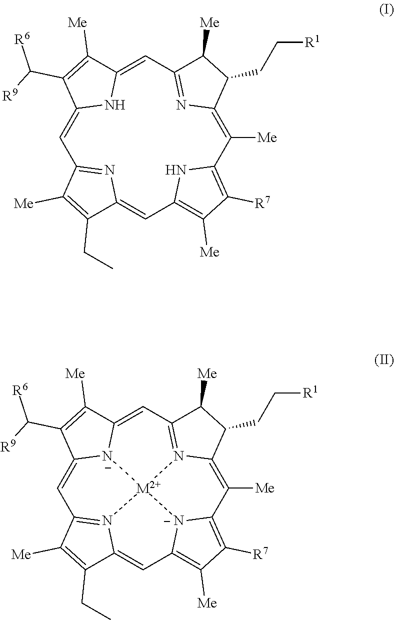

or a pharmaceutically acceptable salt thereof, wherein:
—R¹ is selected from:
(a) —C(O)—OR³, —C(O)—SR³ or —C(O)—N(R³)₂, and —R³, each independently, is —R^{β}, and —R^{β} is a C₁-C₄ alkyl group, more preferably, —R¹ is —C(O)—OR³ and —R³ is —R^{β}, and —R^{β} is a C₁-C₄ alkyl group; or
(b) —C(O)—OR³, —C(O)—SR³ or —C(O)—N(R³)(R^{3′}), —R³ is selected from —R^{α}—OR^{β} or —R^{α}—SR^{β} and —R^{β} is a saccharidyl group, and —R^{3′} is H or C₁-C₄ alkyl;
—R⁶ is selected from —OR² or —SR², and —R² is selected from —R^{α}—OR^{β} or —R^{α}—SR^{β}, and —R^{β} is a saccharidyl group;
—R⁷ is selected from:
(a) —C(O)—OR³, —C(O)—SR³ or —C(O)—N(R³)₂, and —R³, each independently, is C₁-C₄ alkyl, preferably —R⁷ is —C(O)—OR³ and —R³ is C₁-C₄ alkyl; or
(b) —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R⁹ is hydrogen or methyl (preferably —R⁹ is hydrogen); and
M²⁺ is a metal cation.

In a particularly preferred embodiment, the first or second aspect of the present invention provides a compound of formula (I) or a complex of formula (II):

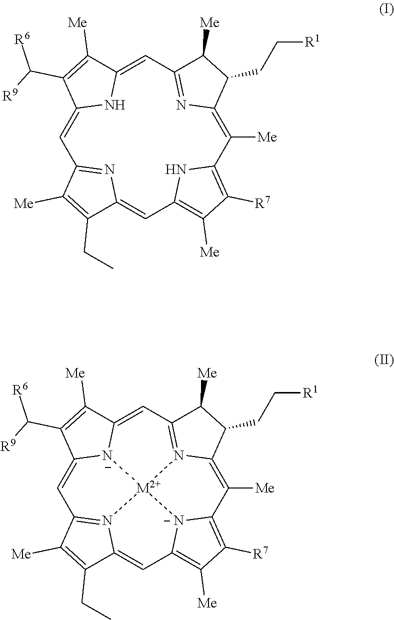

or a pharmaceutically acceptable salt thereof, wherein:
—R¹ is selected from —C(O)—OR³, —C(O)—SR³ or —C(O)—N(R³)₂, and —R³, each independently, is —R^{β}, and —R^{β} is a C₁-C₄ alkyl group, more preferably, —R¹ is —C(O)—OR³ and R³ is —R^{β}, and —R^{β} is a C₁-C₄ alkyl group;
—R⁶ is selected from —OR² or —SR², and —R² is selected from —R^{α}—OR^{β} or —R^{α}—SR^{β}, and —R^{β} is a saccharidyl group;
—R⁷ is selected from:
(a) —C(O)—OR³, —C(O)—SR³ or —C(O)—N(R³)₂, and —R³, each independently, is C₁-C₄ alkyl, preferably —R⁷ is —C(O)—OR³ and —R³ is C₁-C₄ alkyl; or
(b) —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R⁹ is hydrogen or methyl (preferably —R⁹ is hydrogen); and
M²⁺ is a metal cation.

In a particularly preferred embodiment, the first or second aspect of the present invention provides a compound of formula (I) or a complex of formula (II):

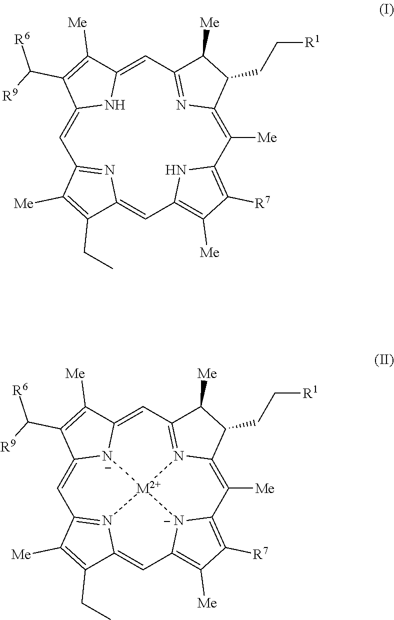

or a pharmaceutically acceptable salt thereof, wherein:
—R¹ is selected from:
(a) —C(O)—OR³, —C(O)—SR³ or —C(O)—N(R³)₂, and —R³, each independently, is —R^{β}, and —R^{β} is a C₁-C₄ alkyl group, more preferably, —R¹ is —C(O)—OR³ and —R³ is —R^{β}, and —R^{β} is a C₁-C₄ alkyl group; or
(b) —C(O)—OR³, —C(O)—SR³ or —C(O)—N(R³)(R^{3′}), —R³ is selected from —R^{α}—OR^{β} or —R^{α}—SR^{β} and —R^{β} is a saccharidyl group, and —R^{3′} is H or C₁-C₄ alkyl;
—R⁶ is selected from —OR² or —SR², and —R² is selected from —R^{α}—OR^{β} or —R^{α}—SR^{β}, and —R^{β} is a saccharidyl group;
—R⁷ is selected from —C(O)—OR³, —C(O)—SR³ or —C(O)—N(R³)₂, and —R³, each independently, is C₁-C₄ alkyl, preferably —R⁷ is —C(O)—OR³ and —R³ is C₁-C₄ alkyl;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R⁹ is hydrogen or methyl (preferably —R⁹ is hydrogen); and
M²⁺ is a metal cation.

In a particularly preferred embodiment, the first or second aspect of the present invention provides a compound of formula (I) or a complex of formula (II):

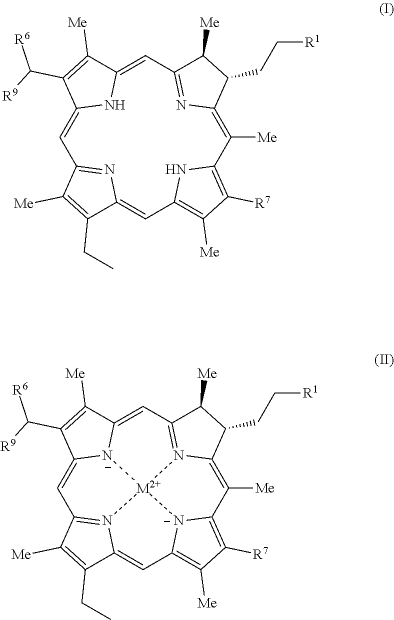

or a pharmaceutically acceptable salt thereof, wherein:
—R¹ is selected from —CH₂OR², —CH₂SR², —CH₂S(O)R², —CH₂S(O)₂R², —CH₂N(R²)(R^{2′}), —R², —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR³, —C(O)—SR³, —C(O)—N(R³)(R^{3′}), —C(S)—OR³, —C(S)—SR³ or —C(S)—N(R³)(R^{3′}); more preferably —R¹ is —C(O)—N(R³)(R^{3′})];
—R², each independently, is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
wherein at least one of —R², —R³ and —R⁴ is selected from —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R^{2′}, —R^{3′} and —R^{4′}, each independently, is selected from hydrogen or C₁-C₆ alkyl [preferably —R^{2′}, —R^{3′} and —R^{4′}, each independently, is selected from hydrogen or C₁-C₃ alkyl; more preferably —R^{2′}, —R^{3′} and —R^{4′}, each independently, is selected from hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X [preferably —R⁶ is selected from —OR², —SR², —S(O)R², —S(O)₂R², or —X];
—R⁷ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R⁷ is —C(O)—N(R³)(R^{3′})];
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is hydrogen or methyl [preferably —R⁹ is hydrogen];
Q is O, S, NH or NMe [preferably Q is O];
X is a halo group;
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In a particularly preferred embodiment, the first or second aspect of the present invention provides a compound of formula (I) or a complex of formula (II):

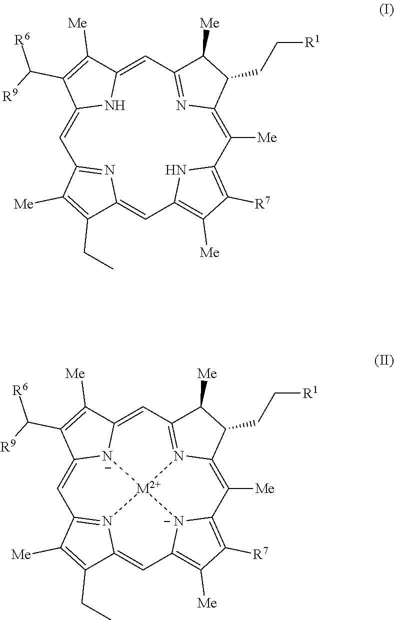

or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R¹ is —C(O)—N(R³)(R^{3′})];
—R² is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
wherein at least one of —R², —R³ and —R⁴ is selected from —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R^{3′} and —R^{4′}, each independently, is selected from hydrogen or C₁-C₃ alkyl [preferably —R^{3′} and —R^{4′}, each independently, is selected from hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₃ alkyl or phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{5′} is selected from C₁-C₃ alkyl substituted with —CO₂ or phenyl substituted with —CO₂, wherein the phenyl may optionally be further substituted with one, two, three or four substituents independently selected from C₁-C₆ alkyl, —O(C₁-C₆ alkyl), —CO₂H, —CO₂Z, —CO₂NH₂, —O—(CH₂CH₂)ₙ—H or —O—(CH₂CH₂)ₙ—CH₃;
—R⁶ is selected from —OR², —N(R²)₂, —SR², —S(O)R², —S(O)₂R², or —X [preferably —R⁶ is selected from —OR², —SR², —S(O)R², —S(O)₂R², or —X];
—R⁷ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R⁷ is —C(O)—N(R³)(R^{3′})];
—R⁸ is —[NC₅H₅] optionally substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{8′} is —[NC₅H₅] substituted with —CO₂ and optionally further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁹ is hydrogen or methyl [preferably —R⁹ is hydrogen];
Q is O, S, NH or NMe [preferably Q is O];
X is a halo group;
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In these two preferred embodiments of the preceding paragraphs, each —R⁵ may be the same or different; preferably each —R⁵ is the same.

In another preferred embodiment of the first or second aspect of the present invention, the compound is a compound of formula (IA), (IB), (IC), (ID), (IE), (IF), (IG), (IH), (IJ), (IK), (IL), (IM), (IN), (IO), (IP), (IQ), (IR), (IS), (IT), (IU) or (IV):

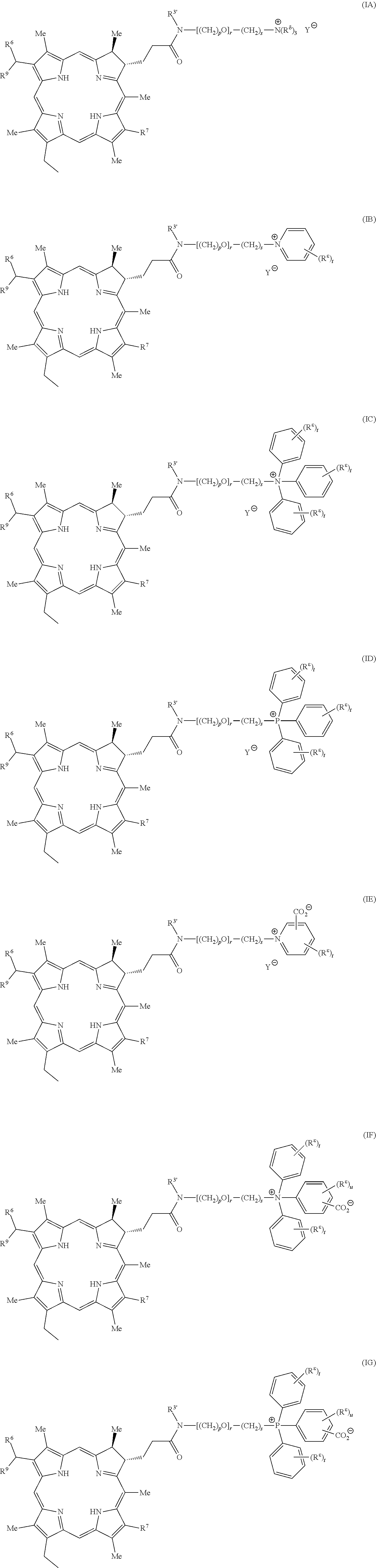

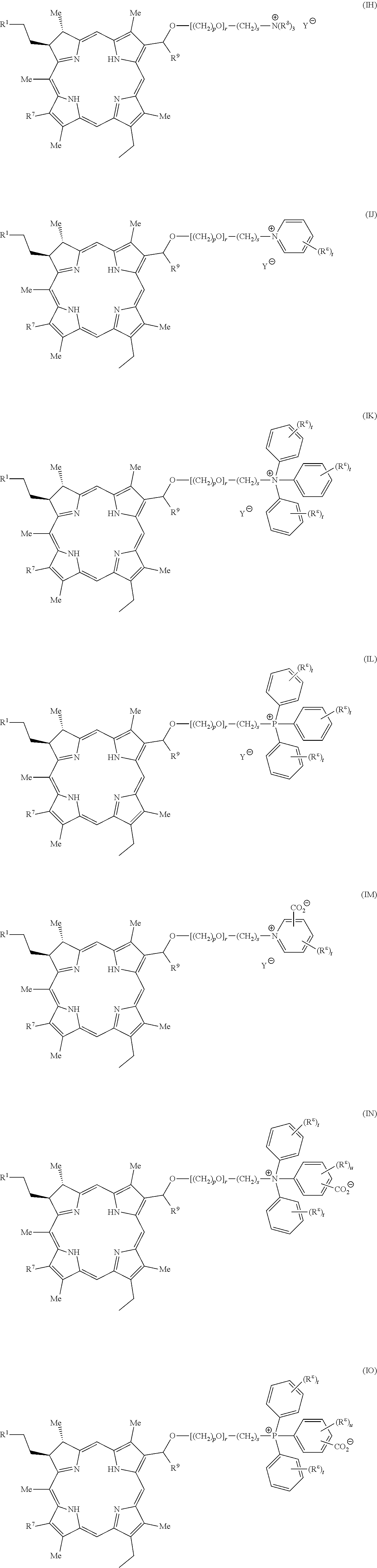

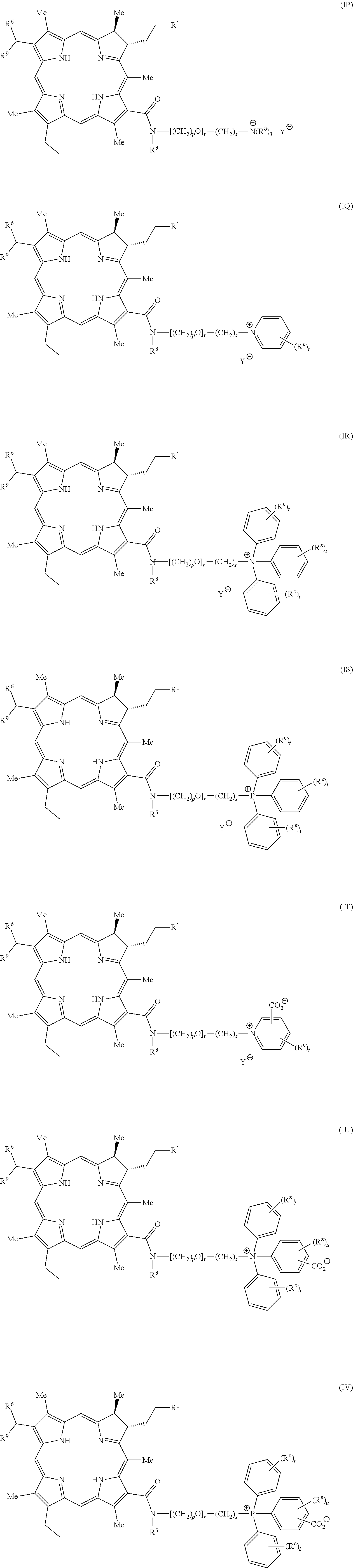

or a metal cation complex thereof, or a pharmaceutically acceptable salt thereof,
wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′});
—R² is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, or —R^{α}—X;
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, or —R^{α}—X;
—R^{3′} and —R^{4′}, each independently, is selected from hydrogen or C₁-C₃ alkyl [preferably —R^{3′} and —R^{4′}, each independently, is selected from hydrogen or methyl];
—R⁶ is selected from —OR², —N(R²)₂, —SR², —S(O)R², —S(O)₂R², or —X [preferably —R⁶ is selected from —OR², —SR², —S(O)R², —S(O)₂R², or —X];
—R⁷ is selected from —C(O)—OR³, —C(O)—SR³, —C(O)—N(R³)(R^{3′}), —C(S)—OR³, —C(S)—SR³ or —C(S)—N(R³)(R^{3′});
—R⁹ is hydrogen or methyl [preferably —R⁹ is hydrogen];
—R^{α}—, each independently, is selected from a C₁-C₁₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R^{δ} is selected from C₁-C₃ alkyl;
—R^{ε} is selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
X is a halo group;
Y is a counter anion;
Z is a counter cation;
n is 1, 2, 3 or 4;
p is 0, 1, 2, 3 or 4;
r is 0, 1, 2, 3, 4, 5 or 6;
s is 0, 1, 2, 3, 4, 5, 6, 7, 8, 9, 10, 11 or 12;
t is 0, 1, 2, 3, 4 or 5; and
u is 0, 1, 2, 3 and 4.

The compounds of formula (IA), (IB), (IC), (ID), (IE), (IF), (IG), (IH), (IJ), (IK), (IL), (IM), (IN), (IO), (IP), (IQ), (IR), (IS), (IT), (IU), (IV) and complexes and salts thereof according to the first and second aspect of the present invention comprise a moiety —[(CH₂)ₚO]ᵣ—(CH₂)ₛ, wherein:
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In one embodiment, p is 2, 3 or 4; r is 1; and s is 2, 3 or 4. In a preferred embodiment, p is 3; r is 1; and s is 3; such that —[(CH₂)ₚO]ᵣ—(CH₂)ₛ is —(CH₂)₃—O—(CH₂)₃—.

In another embodiment, p is 2 or 3; r is 2 or 3; and s is 2 or 3. In a preferred embodiment, p is 2; r is 2; and s is 2; such that —[(CH₂)ₚO]ᵣ—(CH₂)ₛ— is —(CH₂CH₂O)₂—(CH₂)₂—.

In yet another embodiment, r is 0; and s is 1, 2, 3, 4, 5, 6, 7, 8, 9, 10, 11 or 12; such that —[(CH₂)ₚO]ᵣ(CH₂)ₛ— is —(CH₂)₁₋₁₂—.

In another preferred embodiment, the first or second aspect of the present invention provides a compound of formula (I′) or a complex of formula (II′):

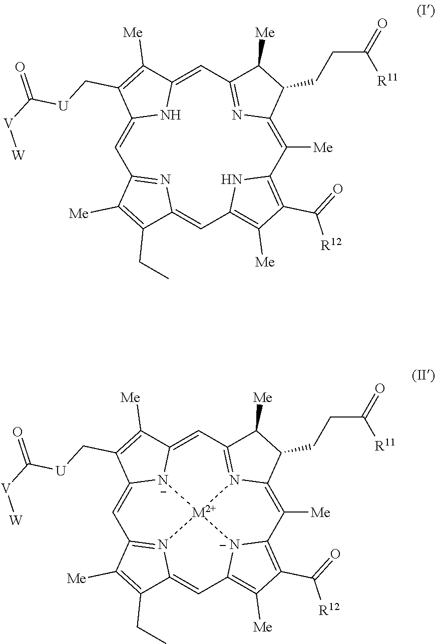

or a pharmaceutically acceptable salt thereof, wherein:
—U— is —O—, —N(R^{u})— or —S—;
—V— is —CH₂—, —O—, —N(R^{v})— or —S—;
—W— is —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R¹¹ and —R¹², each independently, is selected from —OH or —O—(C₁-C₄ alkyl);
—R^{α}— is selected from a C₁-C₁₂ alkylene group, wherein the alkylene group may optionally be substituted with one or more (such as one, two, three or four) C₁-C₄ alkyl, C₁-C₄ haloalkyl or halo groups, and wherein one or more (such as one, two, three or four) carbon atoms in the backbone of the alkylene group may optionally be replaced by a heteroatom or group independently selected from O, S, NH or NMe;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁸ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂)₁—H or —O—(CH₂CH₂O)₁—CH₃ groups;
—R^{8′} is —[NC₅H₅] substituted with —CO₂ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{u} is hydrogen or C₁-C₄ alkyl;
—R^{v} is hydrogen or C₁-C₄ alkyl;
n is 1, 2, 3, 4, 5 or 6;
Y is a counter anion;
Z is a counter cation; and
M²⁺ is a metal cation.

In another preferred embodiment, the first or second aspect of the present invention provides a compound of formula (I′) or a complex of formula (II′):

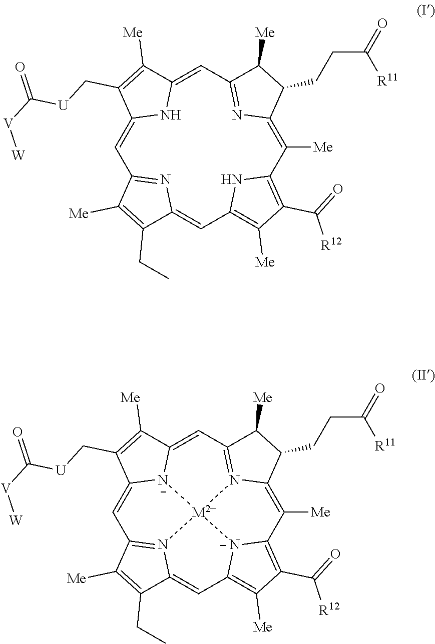

or a pharmaceutically acceptable salt thereof, wherein:
—U— is —O—, —N(R^{u})— or —S—;
—V— is —CH₂—, —O—, —N(R^{v})— or —S—;
—W— is —R^{α}—[N(R⁵)₃]Y, —R^{α}—[P(R⁵)₃]Y or —R^{α}—[R⁸]Y
—R¹¹ and —R¹², each independently, is selected from —OH or —O—(C₁-C₄ alkyl);
—R^{α}— is selected from a C₁-C₁₂ alkylene group (preferably a C₁-C₉ alkylene group, preferably a C₂-C₆ alkylene group), wherein one or more (such as one, two, three or four, preferably one or two) carbon atoms in the backbone of the alkylene group may optionally be replaced by a heteroatom or group independently selected from O, S, NH or NMe (preferably O, NH or NMe, preferably O);
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl) or —O(C₁-C₄ haloalkyl);
—R⁸ is —[NC₅H₅] optionally substituted with one or more C₁-C₄ alkyl, C₁-C₄ haloalkyl, —O(C₁-C₄ alkyl) or —O(C₁-C₄ haloalkyl);
—R^{u} is hydrogen or C₁-C₄ alkyl;
—R^{v} is hydrogen or C₁-C₄ alkyl;
n is 1, 2, 3, 4, 5 or 6;
Y is a counter anion; and
M²⁺ is a metal cation.

Preferably in the compound or complex according to the first or second aspect of the present invention, the compound or complex is:

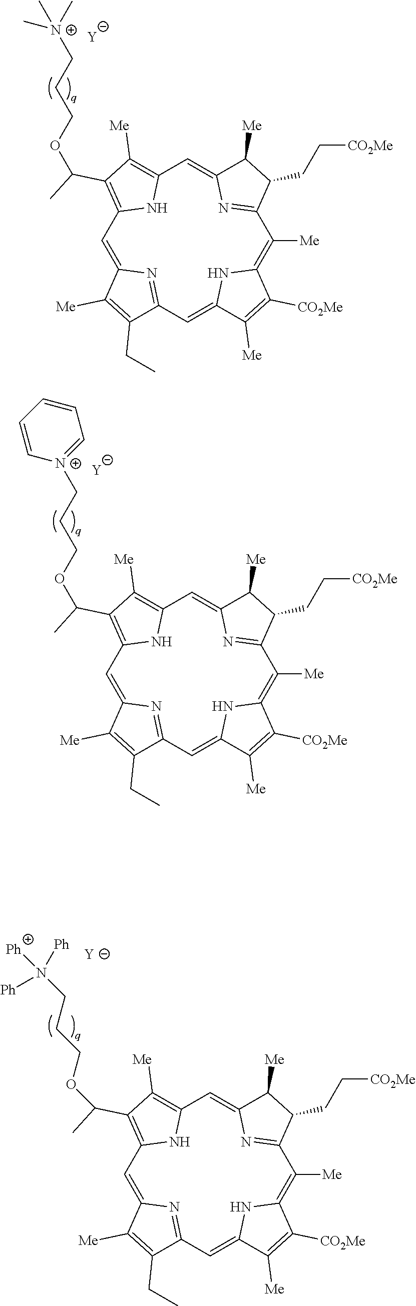

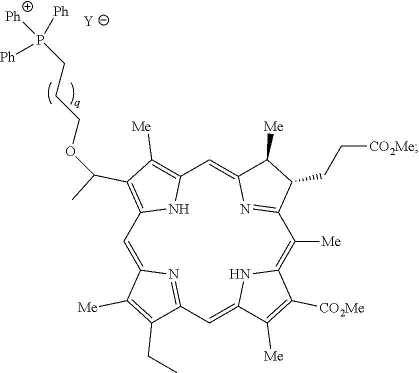

wherein Y is a counter anion, and q is 0, 1, 2, 3 or 4 (preferably q is 1);
or a metal cation complex thereof, or a pharmaceutically acceptable salt thereof.

Preferably in the compound or complex according to the first or second aspect of the present invention, the compound or complex is:

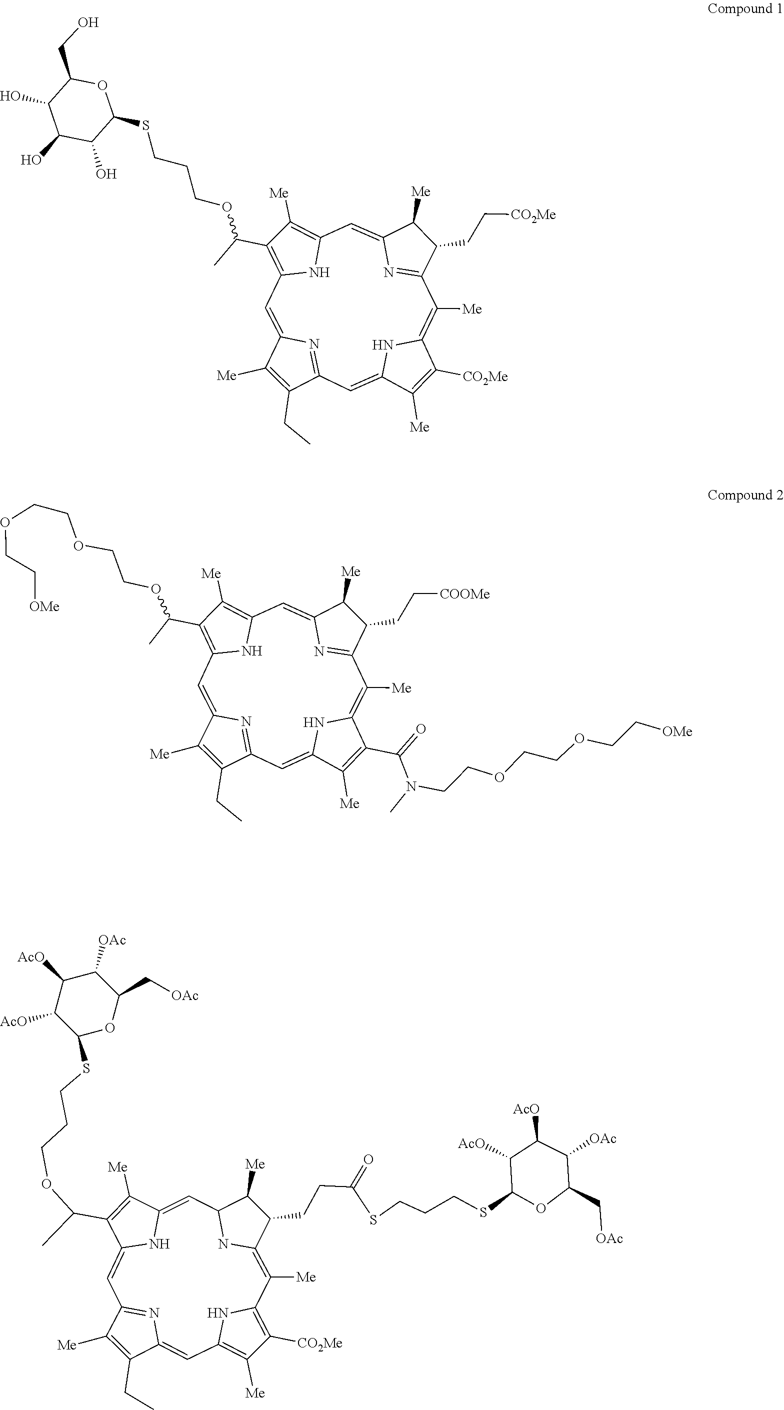

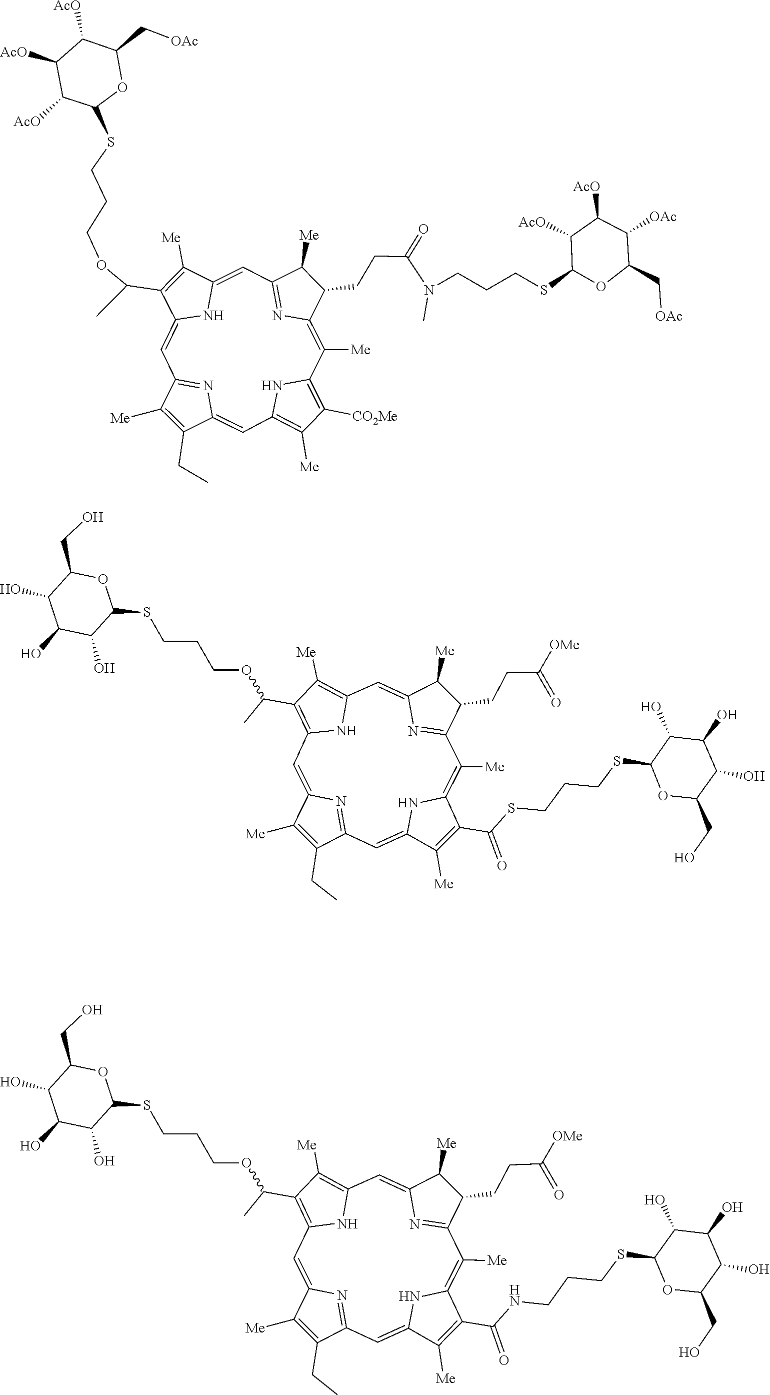

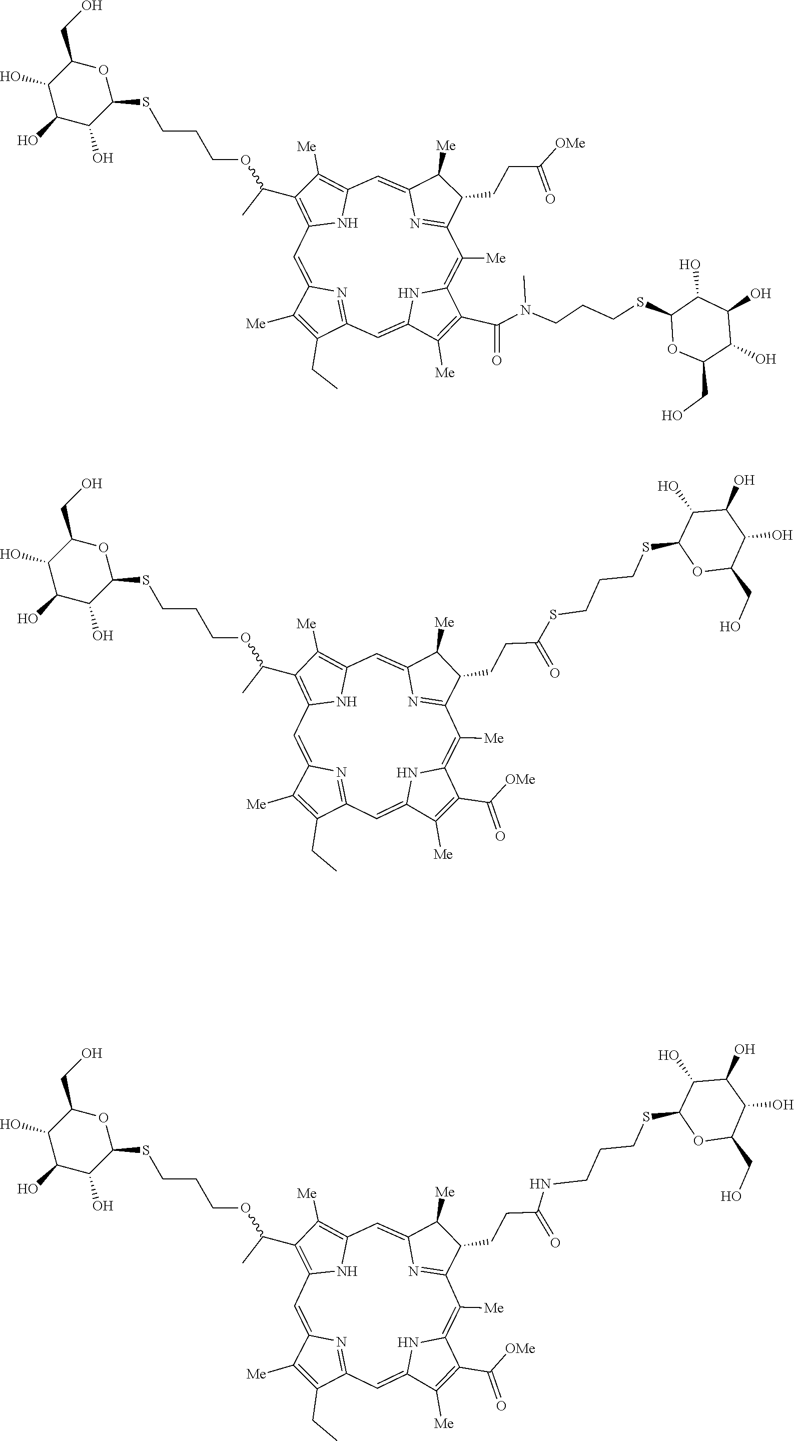

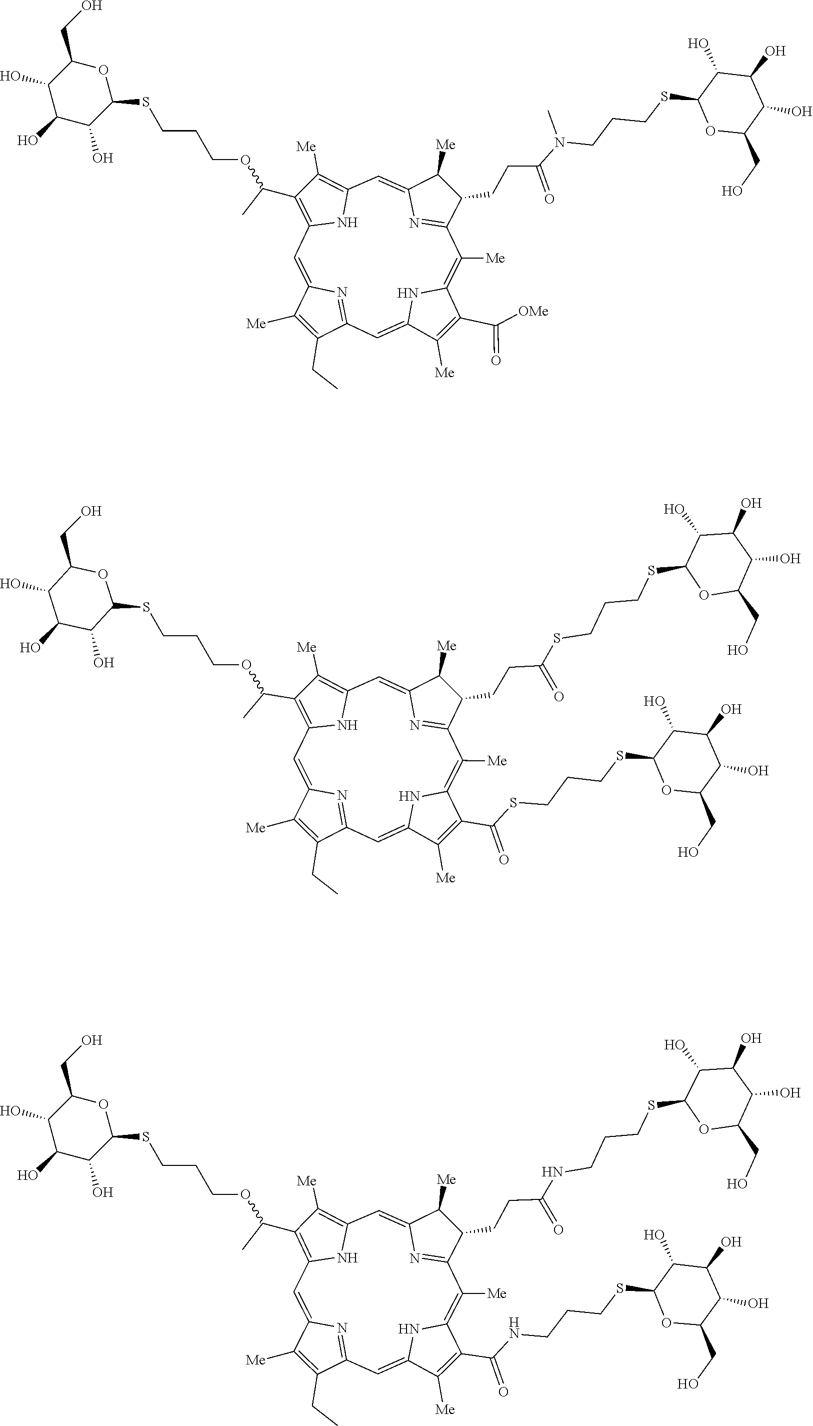

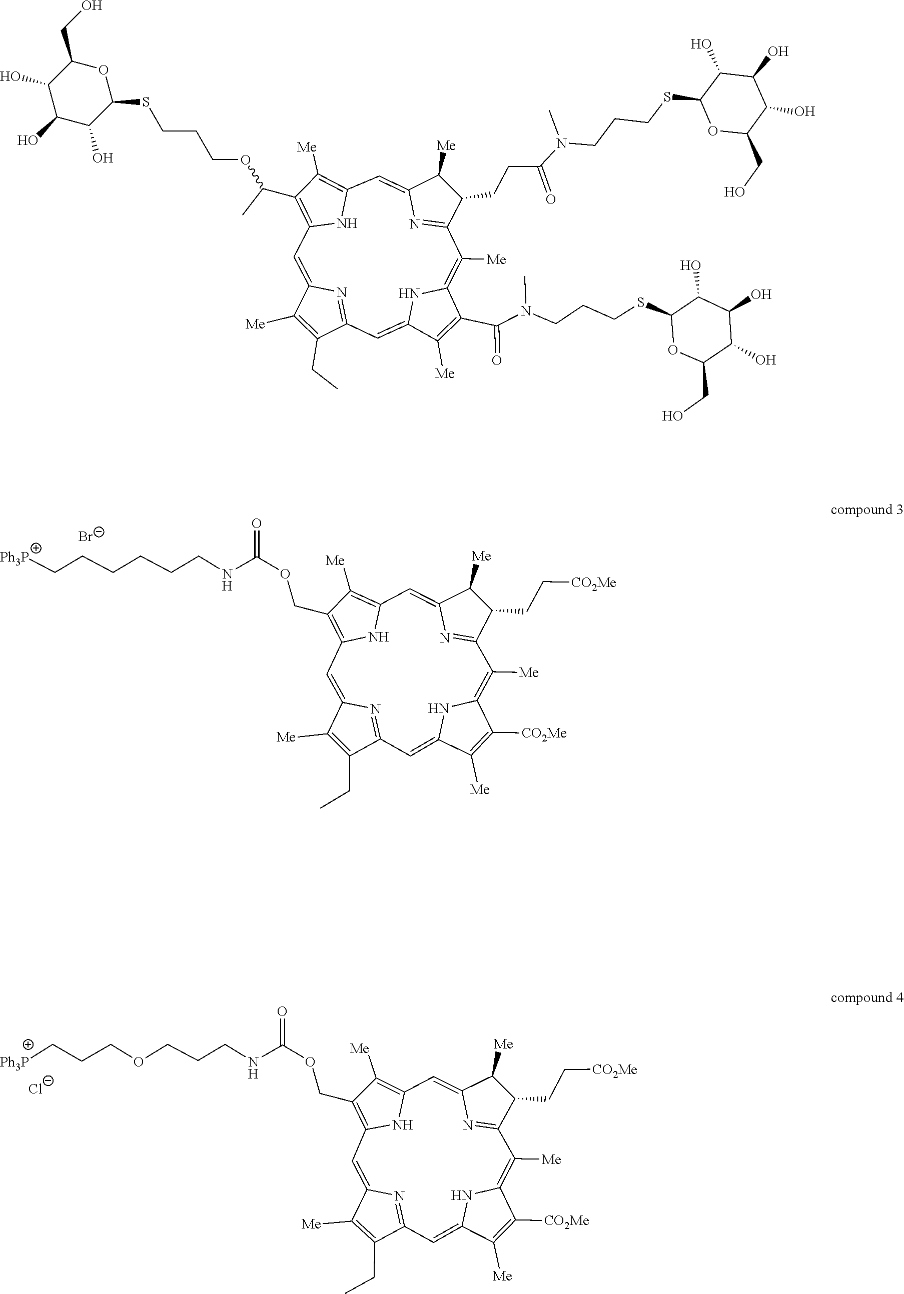

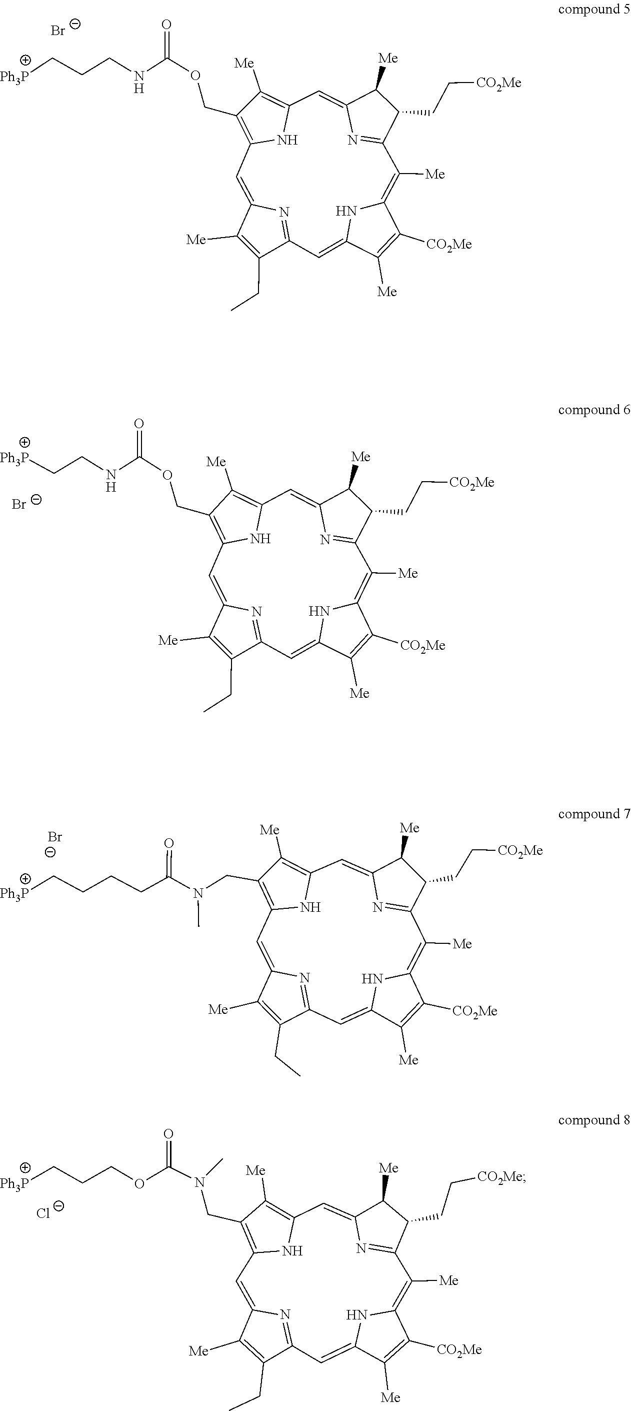

or a metal cation complex thereof, or a pharmaceutically acceptable salt thereof.

In one embodiment, the compound or complex according to the first or second aspect of the invention is in the form of a pharmaceutically acceptable salt. In one embodiment, the compound or complex is in the form of an inorganic salt such as a lithium, sodium, potassium, magnesium, calcium or ammonium salt. In one embodiment, the compound or complex is in the form of a sodium or potassium salt. In one embodiment, the compound is in the form of a sodium salt. In another embodiment, the compound or complex is in the form of an organic salt such as an amine salt (for example a choline or meglumine salt) or an amino acid salt (for example an arginine salt).

The compound or complex according to the first or second aspect of the invention has at least two chiral centres. The compound or complex of the first or second aspect of the invention is preferably substantially enantiomerically pure, which means that the compound or complex comprises less than 10% of other stereoisomers, preferably less than 5%, preferably less than 3%, preferably less than 2%, preferably less than 1%, preferably less than 0.5%, all by weight, as measured by XRPD or SFC.

Preferably, the compound or complex according to the first or second aspect of the invention has a HPLC purity of more than 97%, more preferably more than 98%, more preferably more than 99%, more preferably more than 99.5%, more preferably more than 99.8%, and most preferably more than 99.9%. As used herein the percentage HPLC purity is measured by the area normalisation method.

A third aspect of the invention provides a composition comprising a compound or complex according to the first or second aspect of the invention and a pharmaceutically acceptable carrier or diluent.

In one embodiment, the composition according to the third aspect of the invention further comprises polyvinylpyrrolidone (PVP). In one embodiment, the composition comprises 0.01-10% w/w PVP as percentage of the total weight of the composition, preferably 0.1-5% w/w PVP as a percentage of the total weight of the composition, preferably 0.5-5% w/w PVP as a percentage of the total weight of the composition. In one embodiment, the PVP is K30.

In one embodiment, the composition according to the third aspect of the invention further comprises dimethylsulfoxide (DMSO). In one embodiment, the composition comprises 0.01-99% w/w DMSO as percentage of the total weight of the composition, preferably 40-99% w/w DMSO as a percentage of the total weight of the composition, preferably 65-99% w/w DMSO as a percentage of the total weight of the composition.

In one embodiment, the composition according to the third aspect of the invention further comprises an immune checkpoint inhibitor. In one embodiment, the immune checkpoint inhibitor is an inhibitor of PD-1 (programmed cell death protein 1), PD-L1 (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably the compound or complex according to the first or second aspect of the present invention and the pharmaceutical composition according to the third aspect of the present invention are suitable for use in photodynamic therapy or cytoluminescent therapy.

Preferably the compound or complex according to the first or second aspect of the present invention and the pharmaceutical composition according to the third aspect of the present invention are suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the treatment of a disease characterised by benign or malignant cellular hyperproliferation or by areas of neovascularisation.

Preferably the compound or complex according to the first or second aspect of the present invention and the pharmaceutical composition according to the third aspect of the present invention are suitable for the treatment of a benign or malignant tumour.

Preferably the compound or complex according to the first or second aspect of the present invention and the pharmaceutical composition according to the third aspect of the present invention are suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, so breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for use in photodynamic diagnosis.

Preferably the compound or complex according to the first or second aspect of the present invention and the pharmaceutical composition according to the third aspect of the present invention are suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the detection of an area that is affected by benign or malignant cellular hyperproliferation or by neovascularisation.

Preferably the compound or complex according to the first or second aspect of the present invention and the pharmaceutical composition according to the third aspect of the present invention are suitable for the detection of a benign or malignant tumour.

Preferably the compound or complex according to the first or second aspect of the present invention and the pharmaceutical composition according to the third aspect of the present invention are suitable for the detection of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the fluorescent or phosphorescent detection of the diseases listed above, preferably for the fluorescent or phosphorescent detection and quantification of the said diseases.

Preferably the compound or complex according to the first or second aspect of the present invention and the pharmaceutical composition according to the third aspect of the present invention are adapted for administration simultaneous with or prior to administration of irradiation or sound, preferably for administration prior to administration of irradiation.

If the compound or complex according to the first or second aspect of the present invention or the pharmaceutical composition according to the third aspect of the present invention are for use in photodynamic therapy or cytoluminescent therapy, then they are preferably adapted for administration 5 to 100 hours before the irradiation, preferably 6 to 72 hours before the irradiation, preferably 24 to 48 hours before the irradiation.

If the compound or complex according to the first or second aspect of the present invention or the pharmaceutical composition according to the third aspect of the present invention are for use in photodynamic diagnosis, then they are preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

The pharmaceutical composition according to the third aspect of the present invention may be in a form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration. The pharmaceutical composition may also be in a form suitable for administration by enema or for administration by injection into a tumour. Preferably the pharmaceutical composition is in a form suitable for oral, parenteral (such as intravenous, intraperitoneal, and intratumoral) or airway administration, preferably in a form suitable for oral or parenteral administration, preferably in a form suitable for oral administration.

In one preferred embodiment, the pharmaceutical composition is in a form suitable for oral administration. Preferably the pharmaceutical composition is provided in the form of a tablet, capsule, hard or soft gelatine capsule, caplet, troche or lozenge, as a powder or granules, or as an aqueous solution, suspension or dispersion. More preferably the pharmaceutical composition is provided in the form of an aqueous solution, suspension or dispersion for oral administration, or alternatively in the form of a freeze-dried powder which can be mixed with water before administration to provide an aqueous solution, suspension or dispersion for oral administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for parenteral administration. Preferably the pharmaceutical composition is in a form suitable for intravenous administration. Preferably the pharmaceutical composition is provided in the form of an aqueous solution for parenteral administration, or alternatively in the form of a freeze-dried powder which can be mixed with water before administration to provide an aqueous solution for parenteral administration.

Preferably the pharmaceutical composition is an aqueous solution or suspension having a pH of from 6 to 8.5. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for airway administration. Preferably the pharmaceutical composition is provided in the form of an aqueous solution, suspension or dispersion for airway administration, or alternatively in the form of a freeze-dried powder which can be mixed with water before administration to provide an aqueous solution, suspension or dispersion for airway administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

A fourth aspect of the present invention provides use of a compound or complex according to the first or second aspect of the present invention in the manufacture of a medicament for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a phototherapeutic agent for use in photodynamic therapy or cytoluminescent therapy. Preferably the phototherapeutic agent is suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the medicament or the phototherapeutic agent of the fourth aspect of the present invention is suitable for the treatment of a disease characterised by benign or malignant cellular hyperproliferation or by areas of neovascularisation.

Preferably the medicament or the phototherapeutic agent of the fourth aspect of the present invention is suitable for the treatment of a benign or malignant tumour.

Preferably the medicament or the phototherapeutic agent of the fourth aspect of the so present invention is suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a photodiagnostic agent for use in photodynamic diagnosis.

Preferably the photodiagnostic agent of the fourth aspect of the present invention is suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the detection of an area that is affected by benign or malignant cellular hyperproliferation or by neovascularisation.

Preferably the photodiagnostic agent of the fourth aspect of the present invention is suitable for the detection of a benign or malignant tumour.

Preferably the photodiagnostic agent of the fourth aspect of the present invention is suitable for the detection of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the fluorescent or phosphorescent detection of the said diseases, preferably the fluorescent or phosphorescent detection and quantification of the said diseases.

Preferably the medicament, the phototherapeutic agent or the photodiagnostic agent is adapted for administration simultaneous with or prior to administration of irradiation or sound, preferably for administration prior to administration of irradiation.

If the medicament or the phototherapeutic agent is for use in photodynamic therapy or cytoluminescent therapy, then it is preferably adapted for administration 5 to 100 hours before the irradiation, preferably 6 to 72 hours before the irradiation, preferably 24 to 48 hours before the irradiation.

If the photodiagnostic agent is for use in photodynamic diagnosis, then it is preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

A fifth aspect of the present invention provides a method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of a compound or complex according to the first or second aspect of the present invention to a human or animal in need thereof.

The fifth aspect of the present invention also provides a method of photodynamic therapy or cytoluminescent therapy of a human or animal disease, the method comprising administering a therapeutically effective amount of a compound or complex according to the first or second aspect of the present invention to a human or animal in need thereof. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the method of the fifth aspect of the present invention is a method of treating benign or malignant cellular hyperproliferation or areas of neovascularisation.

Preferably the method of the fifth aspect of the present invention is a method of treating a benign or malignant tumour.

Preferably the method of the fifth aspect of the present invention is a method of treating early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fifth aspect of the present invention also provides a method of photodynamic diagnosis of a human or animal disease, the method comprising administering a diagnostically effective amount of a compound or complex according to the first or second aspect of the present invention to a human or animal. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the human or animal disease is characterised by benign or malignant cellular hyperproliferation or by areas of neovascularisation. Preferably the human or animal disease is a benign or malignant tumour. Preferably the human or animal disease is early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the method of photodynamic diagnosis is suitable for the fluorescent or phosphorescent detection of the said diseases, preferably for the fluorescent or phosphorescent detection and quantification of the said diseases.

In any of the methods of the fifth aspect of the present invention, the human or animal is preferably further subjected to irradiation or sound simultaneous with or after the administration of the compound or complex according to the first or second aspect of the invention. Preferably the human or animal is subjected to irradiation after the administration of the compound or complex according to the first or second aspect of the invention.

If the method is a method of photodynamic therapy or cytoluminescent therapy, then the human or animal is preferably subjected to irradiation 5 to 100 hours after administration of the compound or complex according to the first or second aspect of the invention, preferably 6 to 72 hours after administration, preferably 24 to 48 hours after administration.

If the method is a method of photodynamic diagnosis, then the human or animal is so preferably subjected to irradiation 3 to 60 hours after administration of the compound or complex according to the first or second aspect of the invention, preferably 8 to 40 hours after administration.

Preferably the irradiation is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

In any of the methods of the fifth aspect of the present invention, preferably the human or animal is a human.

A sixth aspect of the present invention provides a pharmaceutical combination or kit comprising:
(a) a compound or complex according to the first or second aspect of the present invention; and
(b) an immune checkpoint inhibitor.

In one embodiment, the immune checkpoint inhibitor is an inhibitor of PD-1 (programmed cell death protein 1), PD-L1 (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably, the combination or kit of the sixth aspect is for use in the treatment of a so disease, disorder or condition, wherein the disease, disorder or condition is responsive to PD-1, PD-L1 or CTLA4 inhibition. Preferably, the combination or kit of the sixth aspect is for use in the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect also provides a use of the combination or kit of the sixth aspect of the invention in the manufacture of a medicament for the treatment of a disease, disorder or condition which is responsive to PD-1, PD-L1 or CTLA4 inhibition. The sixth aspect also provides a use of the combination or kit of the sixth aspect of the invention in the manufacture of a medicament for the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect of the invention also provides a method of treating a disease, disorder or condition which is responsive to PD-1, PD-L1 or CTLA4 inhibition, the method comprising administering a therapeutically effective amount of the combination or kit of the sixth aspect of the present invention to a human or animal in need thereof. The sixth aspect of the invention also provides a method of treating cancer, the method comprising administering a therapeutically effective amount of the combination or kit of the sixth aspect of the present invention to a human or animal in need thereof. In one embodiment, the cancer is melanoma, lung cancer (e.g. non small cell lung cancer), kidney cancer, bladder cancer, head and neck cancer, or Hodgkin's lymphoma.

For the combination or kit of the sixth aspect of the invention, the compound or complex according to the first or second aspect of the invention, and the immune checkpoint inhibitor may be provided together in one pharmaceutical composition or separately in two pharmaceutical compositions. If provided in two pharmaceutical compositions, these may be administered at the same time or at different times.

Preferably the combination or kit of the sixth aspect is adapted for administration simultaneous with or prior to administration of irradiation or sound, preferably for administration prior to administration of irradiation. In one embodiment, the combination or kit of the sixth aspect is adapted for administration 5 to 100 hours before the irradiation, preferably 6 to 72 hours before the irradiation, preferably 24 to 48 hours before the irradiation.

Preferably the irradiation used in the photodynamic therapy or cytoluminescent therapy is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### Synthetic Experimental Details

### Synthesis Example 1—methyl (7S,8S)-18-ethyl-7-(3-methoxy-3-oxopropyl)-2,5,8,12,17-pentamethyl-13-(1-(3-(((2S,3R,4S,5S,6R)-3,4,5-trihydroxy-6-(hydroxymethyl)tetrahydro-2H-pyran-2-yl)thio)propoxy)ethyl)-7H,8H-porphyrin-3-carboxylate (compound 1)

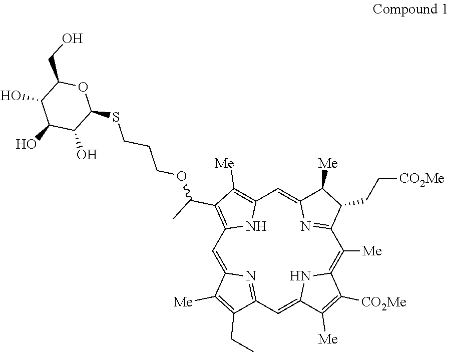 [SMILES: CCC1=C(C)c2cc3[nH]c(cc4nc(c(C)c5[nH]c(cc1n2)c(C)c5C(=O)OC)[C@@H](CCC(=O)OC)[C@@H]4C)c(C)c3C(C)OCCCS[C@@H]1O[C@H](CO)[C@@H](O)[C@H](O)[C@H]1O]

### Synthesis of (2R,3R,4S,5R,6S)-2-(acetoxymethyl)-6-mercaptotetrahydro-2H-pyran-3,4,5-triyl triacetate

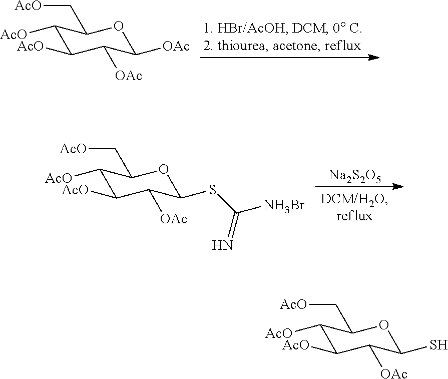

Step 1: A single-neck 100 mL RBF fitted with a 50 mL dropping funnel and nitrogen inlet was charged with 1,2,3,4,6-penta-O-acetyl-β-D-glucose (2.00 g, 5.12 mmol, 1 eq), a stirrer bar and dry DCM (20 mL). The resultant solution was cooled while stirring (420 rpm) under N₂ in an ice-water bath for 10 minutes, before charging the dropping funnel with 33% w/w HBr/AcOH (8.2 mL, 47 mmol, 9.1 eq), and adding it dropwise to the solution over the course of 5 minutes. Following complete addition, the reaction mixture was stirred while being kept in the ice-water bath for 1 hour. The mixture was diluted with Et₂O (100 mL) and washed with H₂O (3×100 mL), then saturated aqueous NaHCO₃ (2×100 mL), before being dried (MgSO₄) and concentrated to give crude 2,3,4,6-tetra-O-acetyl-α-D-glucopyranosyl bromide as a yellow syrup that was used without further purification (2.08 g, 99%).

¹H NMR (400 MHz, CDCl₃) δ 6.61 (d, J=4.1 Hz, 1H), 5.56 (dd, J=9.7, 9.7 Hz, 1H), 5.16 (dd, J=10.1, 9.3 Hz, 1H), 4.83 (dd, J=10.0, 4.1 Hz, 1H), 4.37-4.22 (m, 2H), 4.17-4.08 (m, 1H), 2.10 (s, 3H), 2.10 (s, 3H), 2.05 (s, 3H), 2.03 (s, 3H).

Step 2: The crude glucopyranosyl bromide was dissolved in acetone (20 mL) and added to a 100 mL RBF containing thiourea (0.507 g, 6.66 mmol, 1.3 eq) and 3A molecular sieves (2.0 g). The resultant mixture was refluxed (external temperature=80° C.) while stirring (420 rpm) under N₂ overnight. The product precipitated as a colourless solid. The reaction mixture was diluted with ether (20 mL) and filtered on a glass sinter funnel and washed briefly with ether. The product was separated from the molecular sieves by washing it from the sinter with MeOH and concentrating the filtrate to give pure 2,3,4,6-tetra-O-acetyl-β-D-glucospyranoyl-1-S-isothiouronium bromide as a colourless solid (1.51 g, 60%).

¹H NMR (400 MHz, DMSO) δ 9.16 (br s, 4H), 5.69 (d, J=10.0 Hz, 1H), 5.32 (dd, J=9.4, 9.4 Hz, 1H), 5.16-5.05 (m, 2H), 4.25-4.14 (m, 2H), 4.13-4.03 (m, 1H), 2.06 (s, 3H), 2.02 (s, 3H), 2.00 (s, 3H), 1.97 (s, 3H).

Step 3: To a 100 mL RBF containing 2,3,4,6-tetra-O-acetyl-β-D-glucospyranoyl-1-S-isothiouronium bromide (1.51 g, 3.10 mmol, 1 eq) was added DCM (20 mL) (only partially dissolved), then a solution of Na₂S₂O₅ (2.00 g, 10.5 mmol, 3.4 eq) in dihydrogen monoxide (20 mL). The heterogeneous mixture was refluxed (external temperature=70° C.) for 1 hour, during which time all material dissolved. A concentrated aliquot of the organic layer showed clean conversion to the thiol. The organic phase was separated and washed with H₂O (20 mL), then brine (20 mL), before being dried (MgSO₄) and concentrated by rotary evaporation to give (2R,3R,4S,5R,6S)-2-(acetoxymethyl)-6-mercaptotetrahydro-2H-pyran-3,4,5-triyl triacetate as a colourless oil that solidified upon standing (1.09 g, 96%).

¹H NMR (400 MHz, CDCl₃) δ 5.19 (dd, J=9.9, 9.9 Hz, 1H), 5.09 (dd, J=9.9, 9.9 Hz, 1H), 4.97 (dd, J=9.9, 9.9 Hz, 1H), 4.54 (dd, J=9.9, 9.9 Hz, 1H), 4.25 (dd, J=12.5, 4.8 Hz, 1H), 4.12 (dd, J=12.5, 2.3 Hz, 1H), 3.72 (ddd, J=9.9, 4.8, 2.3 Hz, 1H), 2.31 (d, J=9.9 Hz, 1H), 2.09 (s, 3H), 2.08 (s, 3H), 2.02 (s, 3H), 2.01 (s, 3H).

### Synthesis of methyl (7S,8S)-18-ethyl-7-(3-methoxy-3-oxopropyl)-2,5,8,12,17-pentamethyl-13-vinyl-7H,8H-porphyrin-3-carboxylate

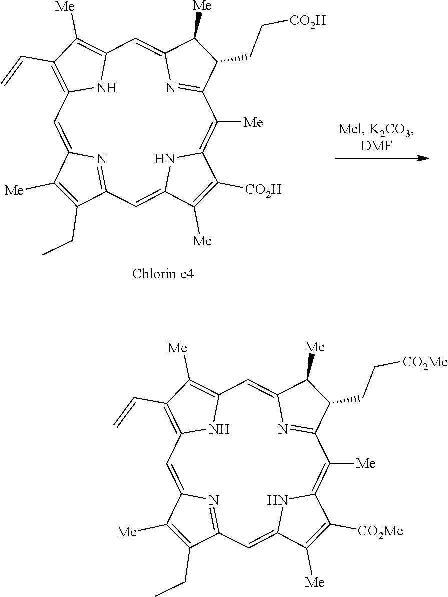 [SMILES: C=Cc1c(C)c2cc3nc(c(C)c4[nH]c(cc5nc(cc1[nH]2)C(C)=C5CC)c(C)c4C(=O)O)[C@@H](CCC(=O)O)[C@@H]3C ; "Chlorin e4" ; C=Cc1c(C)c2cc3nc(c(C)c4[nH]c(cc5nc(cc1[nH]2)C(C)=C5CC)c(C)c4C(=O)OC)[C@@H](CCC(=O)OC)[C@@H]3C]

A single-neck 250 mL RBF was charged with chlorin e4 (5.0 g, 9.05 mmol, 1 eq), potassium carbonate (2.75 g, 19.9 mmol, 2.2 eq) and DMF (100 mL). The resultant solution was stirred (350 rpm) under N₂ before addition of methyl iodide in one portion (1.24 mL, 19.9 mmol, 2.2 eq). The reaction was then stirred overnight monitoring via HPLC. The solvent was removed under reduced pressure and the residual blue solid was then purified via repeated column chromatography (5% MeOH/DCM) to provide the product as a metallic blue solid (3.61 g, 68.8%).

¹H NMR (400 MHz, CDCl₃) δ 9.67 (s, 1H), 9.55 (s, 1H), 8.73 (s, 1H), 8.07 (dd, J=17.9, 11.6 Hz, 1H), 6.34 (dd, J=17.8, 1.5 Hz, 1H), 6.13 (dd, J=11.6, 1.5 Hz, 1H), 4.50-4.40 (m, 2H), 4.30 (s, 3H), 3.81 (s, 3H), 3.79 (q, J=7.6 Hz, 2H), 3.61 (s, 3H), 3.58 (s, 3H), 3.47 (s, 3H), 3.30 (s, 3H), 2.62-2.54 (m, 1H), 2.49-2.39 (m, 1H), 2.26-2.18 (m, 1H), 1.98-1.88 (m, 1H), 1.75 (d, J=7.3 Hz, 3H), 1.71 (t, J=7.6 Hz, 3H), −1.49 (brs, 1H), −1.68 (brs, 1H).

### Synthesis of chlorin e4 3-bromopropyl ether

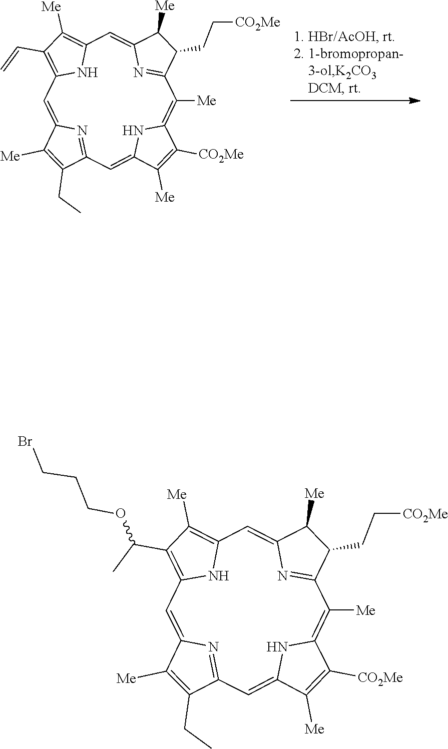 [SMILES: C=Cc1c(C)c2cc3nc(c(C)c4[nH]c(cc5nc(cc1[nH]2)C(C)=C5CC)c(C)c4C(=O)OC)[C@@H](CCC(=O)OC)[C@@H]3C ; CCC1=C(C)c2cc3[nH]c(cc4nc(c(C)c5[nH]c(cc1n2)c(C)c5C(=O)OC)[C@@H](CCC(=O)OC)[C@@H]4C)c(C)c3C(C)OCCCBr]

To a 100 mL RBF containing chlorin e4 dimethyl ester (0.480 g, 0.847 mmol, 1 eq) and a stirrer bar was added HBr/AcOH (33% w/w, 6 mL), and the dark blue mixture stirred (420 rpm) at 30° C. (external temperature) for 2 hours under N₂. ¹H NMR analysis of a concentrated aliquot at this point showed clean conversion to the bromide. A stream of N₂ was passed over the sample for a few minutes to remove some of the HBr before concentrating the bulk by rotary evaporation. The mixture was then further dried under high vacuum (0.2 mbar) at an external temperature of 40° C. for 30 minutes. The residue was reconstituted in DCM (20 mL) before K₂CO₃ (1.17 g, 8.47 mmol, 10 eq), then 3-bromo-propan-1-ol (1.44 mL, 16.9 mmol, 20 eq) were added. The system was flushed with N₂ and the dark solution was stirred overnight at 30° C. (external temperature). A concentrated aliquot of the reaction mixture (high vacuum pump used) showed clean conversion to the ether. The reaction mixture was transferred to a separatory funnel and washed with H₂O (20 mL), then brine (20 mL), before being dried (Na₂SO₄) and concentrated by rotary evaporation to give the crude ether as a dark green oil. The residue was further dried under high vacuum for 30 minutes at an external temperature of 40° C. to try and distil out some of the excess 3-bromo-propan-1-ol, giving a final crude mass of 1.93 g. The residue was purified by column chromatography (25% EtOAc/hexanes, loaded as a solution in the eluent, R_{f}=0.4, visualised by UV) to give chlorin e4 3-bromopropyl ether as a dark green film (302 mg, 51%).

### Synthesis of methyl (7S,8S)-18-ethyl-7-(3-methoxy-3-oxopropyl-2,5,8,12,17-pentamethyl-13-(1-(3-(((2S,3R,4S,5S,6R)-3,4,5-trihydroxy-6-(hydroxymethyl)tetrahydro-2H-pyran-2-yl)thio)propoxy)ethyl)-7H,8H-porphyrin-3-carboxylate (compound 1)

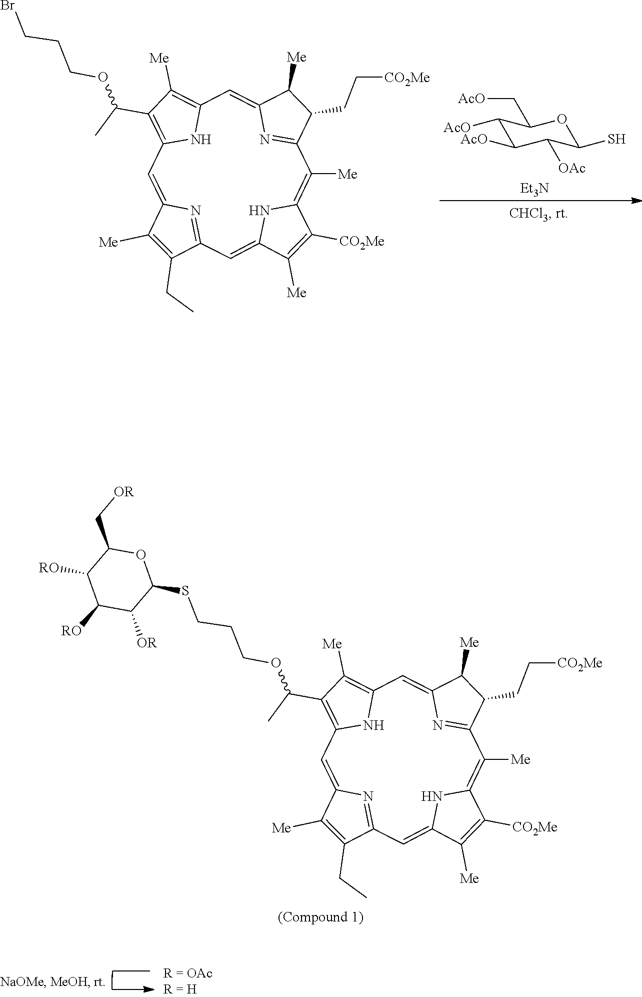

Step 1: To a 50 mL RBF containing chlorin e4 3-bromopropyl ether (0.214 g, 0.297 mmol, 1 eq) was added CHCl₃ (3 mL), (2R,3R,4S,5R,6S)-2-(acetoxymethyl)-6-mercaptotetrahydro-2H-pyran-3,4,5-triyl triacetate (92% w/w, 0.389 g, 0.981 mmol, 3.3 eq), then triethylamine (0.24 mL, 1.8 mmol, 5.9 eq). The dark green mixture was stirred (420 rpm) under N₂ at 30° C. (external temperature) and reaction progress was monitored by TLC (25% EtOAc/hexanes). After stirring overnight, the reaction mixture was washed with H₂O (10 mL). The organic phase was collected and dried (Na₂SO₄) before concentrating by rotary evaporation to give the crude coupled product as a dark green film (677 mg). The residue was purified by column chromatography (0-10% MeOH/DCM) to give chlorin e4 dimethyl ester propyloxythio-D-glucose peracetate (261 mg).

Step 2: To a solution of chlorin e4 dimethyl ester propyloxythio-D-glucose peracetate (0.261 g, 0.260 mmol, 1 eq) in a mixture of DCM (4 mL) and MeOH (4 mL) was added a 4.6 M solution of NaOMe in MeOH (0.57 mL, 2.60 mmol, 10 eq) dropwise. The dark green solution was stirred at ambient temperature for 20 minutes, whereupon reaction completion was verified by TLC (3% MeOH/DCM). The reaction was quenched with AcOH (10 drops) and concentrated by rotary evaporation. The residue was partitioned between DCM (10 mL) and H₂O (10 mL), and the organic phase collected and washed with brine (10 mL). The organic phase was dried (Na₂SO₄) and concentrated by rotary evaporation to give the crude deacetylated product as a dark green film (267 mg). The residue was purified by column chromatography (5-15% MeOH/DCM) to give compound 1 as a dark green foam (92 mg, 37%—over 2 steps).

### Synthesis Example 2—Synthesis of methyl 3-((7S,8S)-18-ethyl-3-((2-(2-(2-methoxyethoxy)ethoxy)ethyl)(methyl)carbamoyl)-2,5,8,12,17-pentamethyl-13-(2,5,8,11-tetraoxatridecan-12-yl)-7H,8H-porphyrin-7-yl)propanoate (compound 2)

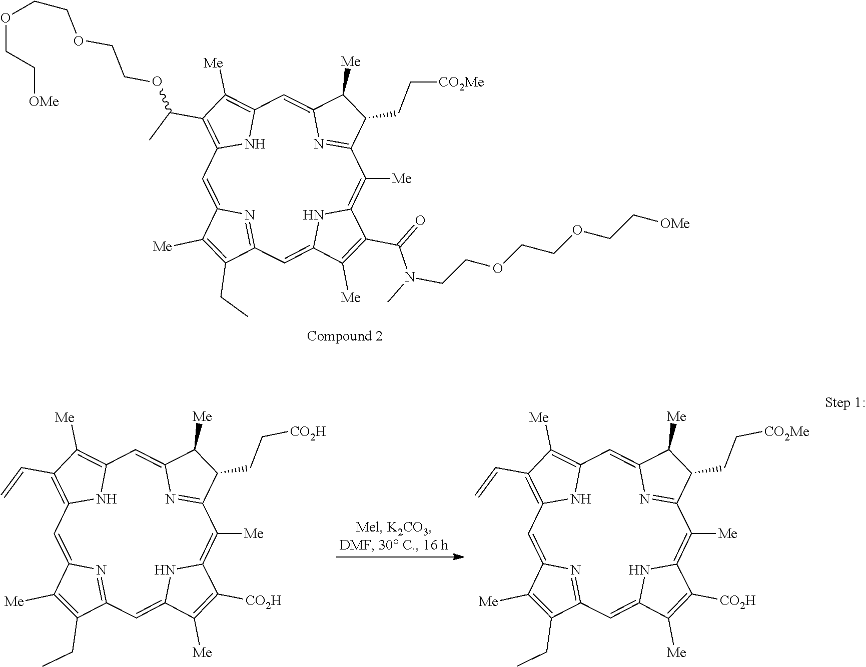 [SMILES: CCC1=C(C)c2cc3[nH]c(cc4nc(c(C)c5[nH]c(cc1n2)c(C)c5C(=O)N(C)CCOCCOCCOC)[C@@H](CCC(=O)OC)[C@@H]4C)c(C)c3C(C)OCCOCCOCCOC ; C=Cc1c(C)c2cc3nc(c(C)c4[nH]c(cc5nc(cc1[nH]2)C(C)=C5CC)c(C)c4C(=O)O)[C@@H](CCC(=O)O)[C@@H]3C ; C=Cc1c(C)c2cc3nc(c(C)c4[nH]c(cc5nc(cc1[nH]2)C(C)=C5CC)c(C)c4C(=O)O)[C@@H](CCC(=O)OC)[C@@H]3C]

A single-neck 250 mL RBF was fitted with a short air condenser and charged with chlorin e4 (1.00 g, 1.81 mmol), potassium carbonate (0.26 g, 1.90 mmol, 1.05 eq) and DMF (130 mL). The resultant solution was stirred (420 rpm) under N₂ before addition of methyl iodide in one portion (0.10 mL, 1.63 mmol, 0.9 eq). The reaction was stirred at 30° C. for 16 hours, allowed to cool to room temperature and then concentrated under reduced pressure. The residual blue solid was analysed by HPLC which indicated that some starting material and dimethyl ester were present. The crude residue was heated to reflux in acetone (40 mL), allowed to cool and then refrigerated overnight. The precipitate was collected via vacuum filtration on filter paper to afford a metallic blue solid (578 mg, 56%). The material was adsorbed to silica gel and purified by column chromatography (1% to 30% MeOH in CH₂Cl₂) to provide the desired product as a metallic blue solid.

¹H NMR (400 MHz, d6-DMSO) 9.74 (s, 1H), 9.65 (s, 1H), 9.05 (s, 1H), 8.29 (dd, J=17.8, 11.6 Hz, 1H), 6.45 (dd, J=17.8, 1.6 Hz, 1H), 6.18 (dd, J=11.6, 1.4 Hz, 1H), 4.59 (q, J=7.1 Hz, 1H), 4.49 (d, J=10.1 Hz, 1H), 4.25 (s, 3H), 3.79 (q, J=7.5 Hz, 2H), 3.74 (s, 3H), 3.54 (s, 3H), 3.50 (s, 3H), 2.70-2.55 (m, 1H), 2.39-2.26 (m, 2H), 1.70 (d, J=7.2 Hz, 3H), 1.67 (t, J=7.6 Hz, 4H), −1.67 (s, 1H), −1.88 (s, 1H).

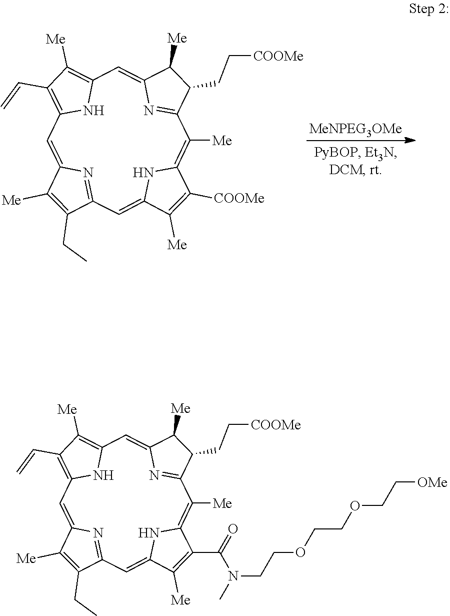 [SMILES: C=Cc1c(C)c2cc3nc(c(C)c4[nH]c(cc5nc(cc1[nH]2)C(C)=C5CC)c(C)c4C(=O)OC)[C@@H](CCC(=O)OC)[C@@H]3C ; C=Cc1c(C)c2cc3nc(c(C)c4[nH]c(cc5nc(cc1[nH]2)C(C)=C5CC)c(C)c4C(=O)N(C)CCOCCOCCOC)[C@@H](CCC(=O)OC)[C@@H]3C]

A 50 mL RBF fitted with a nitrogen inlet was charged with a small stirrer bar, 2-(2-(2-methoxyethoxy)ethoxy)-N-methylethan-1-amine (75 mg, 0.424 mmol, 1.2 eq), chlorin e4 monomethyl ester (87% w/w, 230 mg, 0.353 mmol, 1 eq), triethylamine (147 μL, 1.06 mmol, 3 eq) and DCM (5 mL). To the resultant dark green solution was added PyBOP (202 mg, 0.388 mmol, 1.1 eq), and the mixture was stirred under N₂ for 1 hour. TLC analysis (5% MeOH/DCM, R_{f} (starting material)=0.4, R_{f} (product)=0.45, visualised by UV) at this point indicated clean conversion. The reaction mixture was concentrated by rotary evaporation and the residue purified by Biotage autocolumn chromatography to give the desired product. The product was taken up in EtOAc (20 mL) and washed with H₂O (3×20 mL). The organic phase was dried (Na₂SO₄) and concentrated by rotary evaporation to give methyl 3-((7S,8S)-18-ethyl-3-((2-(2-(2-methoxyethoxy)ethoxy)ethyl)(methyl)carbamoyl)-2,5,8,12,17-pentamethyl-13-vinyl-7H,8H-porphyrin-7-yl)propanoate as a blue/brown film (233 mg, 91%).

¹H NMR (400 MHz, CDCl₃) δ 9.66 (s, 1H), 9.54 (s, 1H), 8.74 (s, 1H), 8.06 (dd, J=17.8, 11.5 Hz, 1H), 6.35 (dd, J=17.8, 1.5 Hz, 1H), 6.13 (dd, J=11.5, 1.6 Hz, 1H), 4.58 (dd, J=8.5, 2.5 Hz, 1H), 4.49 (app. q, J=7.3 Hz, 1H), 4.30 (s, 3H), 3.85 (s, 3H), 3.80 (app. q, J=7.6 Hz, 2H), 3.59 (s, 3H), 3.55-3.49 (m, 4H), 3.48 (s, 3H), 3.47-3.38 (m, 3H), 3.31 (s, 3H), 3.29-3.22 (m, 4H), 3.11-2.90 (m).

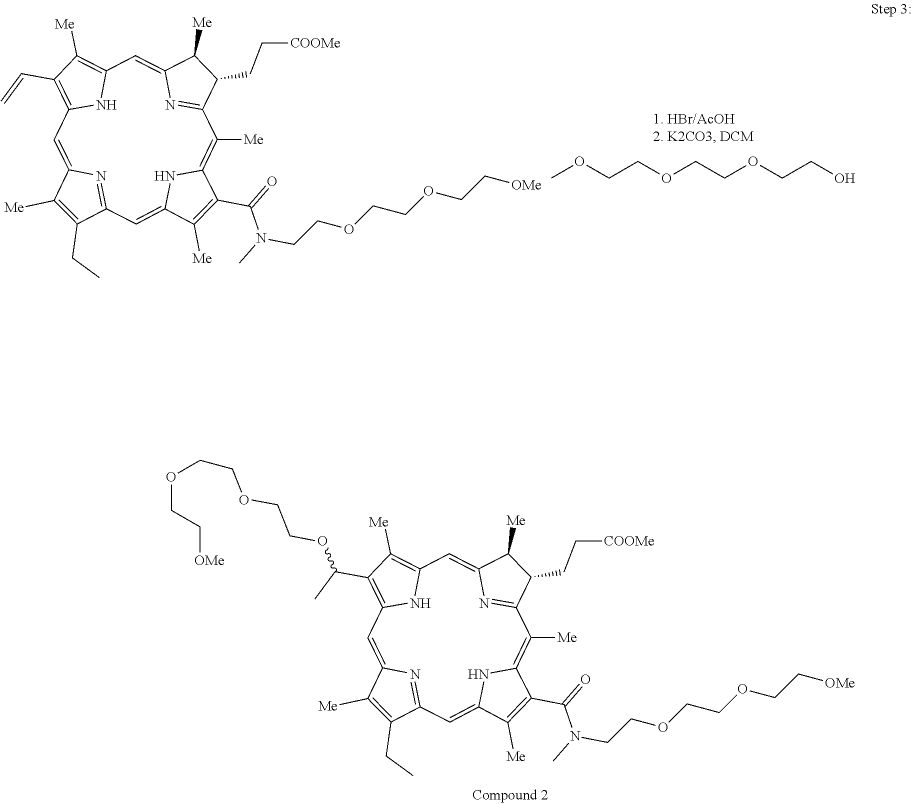

To a 50 mL RBF containing methyl 3-((7S,8S)-18-ethyl-3-((2-(2-(2-methoxyethoxy)ethoxy)ethyl)(methyl)carbamoyl)-2,5,8,12,17-pentamethyl-13-vinyl-7H,8H-porphyrin-7-yl)propanoate (0.50 g, 0.689 mmol, 1 eq) and a stirrer bar was added HBr/AcOH (33% w/w, 5 mL) and the dark blue mixture was stirred (350 rpm) at 30° C. for 2 hours under N₂. A stream of N₂ was passed over the sample for a few minutes to remove some of the HBr before concentrating the bulk by rotary evaporation. The mixture was then further dried under high vacuum (1.5 mbar) with heating (40° C.) for 30 minutes. The residue was reconstituted in DCM (20 mL) before K₂CO₃ (0.95 g, 6.89 mmol, 10 eq), then triethylene glycol monomethyl ether (1.13 g, 6.89 mmol, 10 eq) were added. The dark solution was stirred at 30° C. (external temperature) overnight. HPLC analysis showed 3 main peaks (including the starting material). The reaction mixture was transferred to a separatory funnel, diluted with DCM (20 mL) and washed with H₂O (50 mL), then brine (50 mL) before being dried (Na₂SO₄) and concentrated to give the crude coupled product as a dark green oil (0.80 g). The crude material was purified by silica column chromatography (1-2% MeOH/DCM). Fractions containing the product (R_{f} 0.60 in 5% MeOH/DCM) were combined and purified further using Biotage autocolumn chromatography. The crude product was loaded on as a solution in DCM that had been pre-filtered through a 0.45 μm syringe filter and eluted using 0-5% MeOH to give the product, compound 2, as a dark green viscous oil (260 mg, 42.6%) (HPLC purity: 93.1%).

### Synthesis Example 3—Synthesis of chlorin e4 dimethyl ester 13-(6-(triphenylphosphonium bromide)hexyl)carbamate (compound 3)

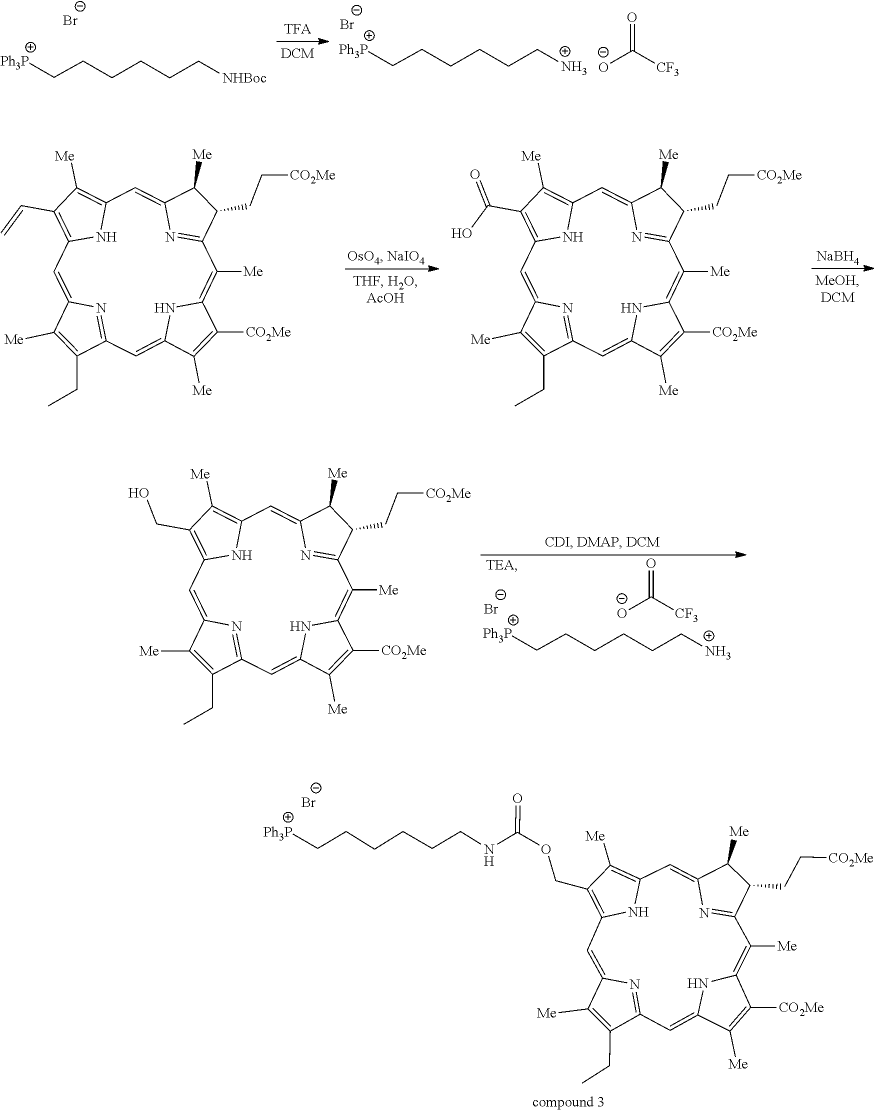

Step 1: To a 50 mL RBF was added (6-((tert-butoxycarbonyl)amino)hexyl) triphenylphosphonium bromide (1.00 g, 1.843 mmol, 9.7 eq), DCM (10 mL) and TFA (2 mL). The resultant solution was stirred (420 rpm) for 1 hour at ambient temperature, then concentrated on the rotary evaporator. The residue was resuspended and concentrated twice from chloroform (2×40 mL) to give 6-aminohexyltriphenylphosphonium bromide TFA as a viscous oil (1.814 g) that was dissolved in DCM (2 mL) for the subsequent coupling reaction.

Step 2: To a 100 mL RBF was added chlorin e4 dimethyl ester (1.98 g, 3.41 mmol, 1 eq), THF (75 mL), osmium tetroxide (8.7 mg, 0.0341 mmol, 0.01 eq), deionized water (6 mL), AcOH (6 mL) and sodium periodate (1.90 g, 8.87 mmol, 2.6 eq). The resultant mixture was stirred (420 rpm) under nitrogen in the dark at ambient temperature for 18 hours. The reaction mixture was concentrated using a rotary evaporator to remove the THF and then re-dissolved in DCM (100 mL), transferred to a separatory funnel and washed with brine (50 mL), saturated NaHCO₃ (50 mL) and water (80 mL) before being dried (Na₂SO₄) and concentrated by rotary evaporation to give a dark blue solid (2.26 g). The crude product was purified by silica gel column chromatography using an eluent gradient of 1.5-4% MeOH in DCM. The first major band (R_{f}=0.7 in 5% MeOH/DCM) was concentrated by rotary evaporation to give chlorin e4 13-formyl dimethyl ester as a dark blue solid (1.18 g, 59%).

¹H NMR (400 MHz, CDCl₃) δ 11.54 (s, 1H), 10.23 (s, 1H), 9.64 (s, 1H), 8.95 (s, 1H), 4.57-4.43 (m, 2H), 4.33 (s, 3H), 3.86 (s, 3H), 3.84-3.72 (m, 6H), 3.64 (s, 3H), 3.60 (s, 3H), 3.32 (s, 3H), 2.64 (dt, J=15.9, 8.0 Hz, 1H), 2.47 (dtd, J=14.1, 8.0, 2.9 Hz, 1H), 2.29 (ddd, J=15.8, 8.2, 4.9 Hz, 1H), 1.97-1.83 (m, 1H), 1.78 (d, J=7.2 Hz, 3H), 1.72 (t, J=7.6 Hz, 3H), 1.57 (s, 1H), −1.50 (s, 1H), −2.01 (s, 1H).

Step 3: To a 100 mL RBF was added chlorin e4 13-formyl dimethyl ester (851 mg, 1.46 mmol, 1 eq), MeOH (20 mL), DCM (10 mL) and sodium borohydride (110 mg, 2.92 mmol, 2 eq). The resultant mixture was stirred (400 rpm) under nitrogen ambient temperature for 1 hour. The reaction mixture was diluted with water (50 mL) and stirred for 10 minutes. The mixture was then diluted with DCM (20 mL) and brine (20 mL). The DCM layer was collected and the aqueous further extracted with DCM (20 mL). The combined DCM layers were washed with brine (30 mL), dried (Na₂SO₄) and concentrated by rotary evaporation to give a dark blue solid (902 mg) that was purified by chromatography. The crude product was dissolved in DCM and eluted using 2-5% MeOH/DCM. Fractions containing the product (major dark green spot, R_{f}=0.6 in 5% MeOH/DCM) were combined to give chlorin e4 13-hydroxymethyl dimethyl ester as a dark blue solid (601 mg, 70%).

¹H NMR (400 MHz, CDCl₃) δ 9.65 (s, 1H), 9.45 (s, 1H), 8.73 (s, 1H), 5.76 (s, 2H), 4.54-4.40 (m, 2H), 4.30 (s, 3H), 3.82 (s, 3H), 3.74 (q, J=7.4 Hz, 2H), 3.60 (s, 3H), 3.57 (s, 3H), 3.39 (s, 3H), 3.26 (s, 3H), 2.58 (dt, J=15.8, 8.0 Hz, 1H), 2.49-2.38 (m, 1H), 2.27-2.15 (m, 1H), 1.93 (dtd, J=14.1, 9.1, 4.9 Hz, 1H), 1.75 (d, J=7.2 Hz, 3H), 1.70 (t, J=7.6 Hz, 3H), −1.64 (s, 1H), −1.83 (s, 1H).

Step 4: To a 50 mL RBF was added chlorin e4 13-hydroxymethyl dimethyl ester (100 mg, 0.171 mmol, 1 eq), carbonyl diimidazole (55 mg, 0.342 mmol, 2 eq), DCM (5 mL) and DMAP (10 mg). The resultant mixture was stirred under nitrogen for 3 hours with monitoring by TLC. TEA (1.17 g, 11.6 mmol) was added followed by 6-aminohexyltriphenylphosphonium bromide (1.814 g, containing ˜1.01 g TFA, in DCM 2 mL) and stirring was continued for 4 days. The reaction mixture was diluted with DCM (20 mL), transferred to a separatory funnel and washed with 1 M HCl (2×25 mL) and pH=7 phosphate buffer (25 mL) before being dried (Na₂SO₄) and concentrated by rotary evaporation to give a dark green residue. The residue was purified by column chromatography (4×18 cm) using 5-8% MeOH/DCM, loaded as a solution in the eluent. Fractions containing the major dark green band (R_{f}=0.30 in 10% MeOH/DCM) were combined and concentrated by rotary evaporation to give compound 3 (106 mg, 59%).

¹H NMR (400 MHz, CDCl₃) δ 9.63 (s, 1H), 9.58 (s, 1H), 8.73 (s, 1H), 7.67-7.51 (m, 9H), 7.51-7.39 (m, 5H), 6.37 (s, 2H), 5.69 (t, J=5.9 Hz, 1H), 4.49-4.38 (m, 2H), 4.29 (s, 3H), 3.80 (s, 3H), 3.75 (q, J=7.9 Hz, 2H), 3.60 (s, 3H), 3.56 (s, 6H), 3.48 (s, 3H), 3.28 (s, 3H), 3.23 (q, J=6.4 Hz, 2H), 2.58 (dt, J=15.9, 8.0 Hz, 1H), 2.42 (ddd, J=14.1, 8.7, 6.0 Hz, 1H), 2.28-2.15 (m, 1H), 1.89 (dtd, J=14.0, 9.0, 4.9 Hz, 1H), 1.77-1.62 (m, 12H), 1.62-1.52 (m, 1H), 1.51-1.39 (m, 1H), 1.39-1.29 (m, 1H), −1.62 (s, 1H), −1.84 (s, 1H).

### Synthesis Example 4—Synthesis of chlorin e4 dimethyl ester 13-(N-(3-(3-triphenylphosphoniumpropoxy)propyl)chloride)carbamate (compound 4)

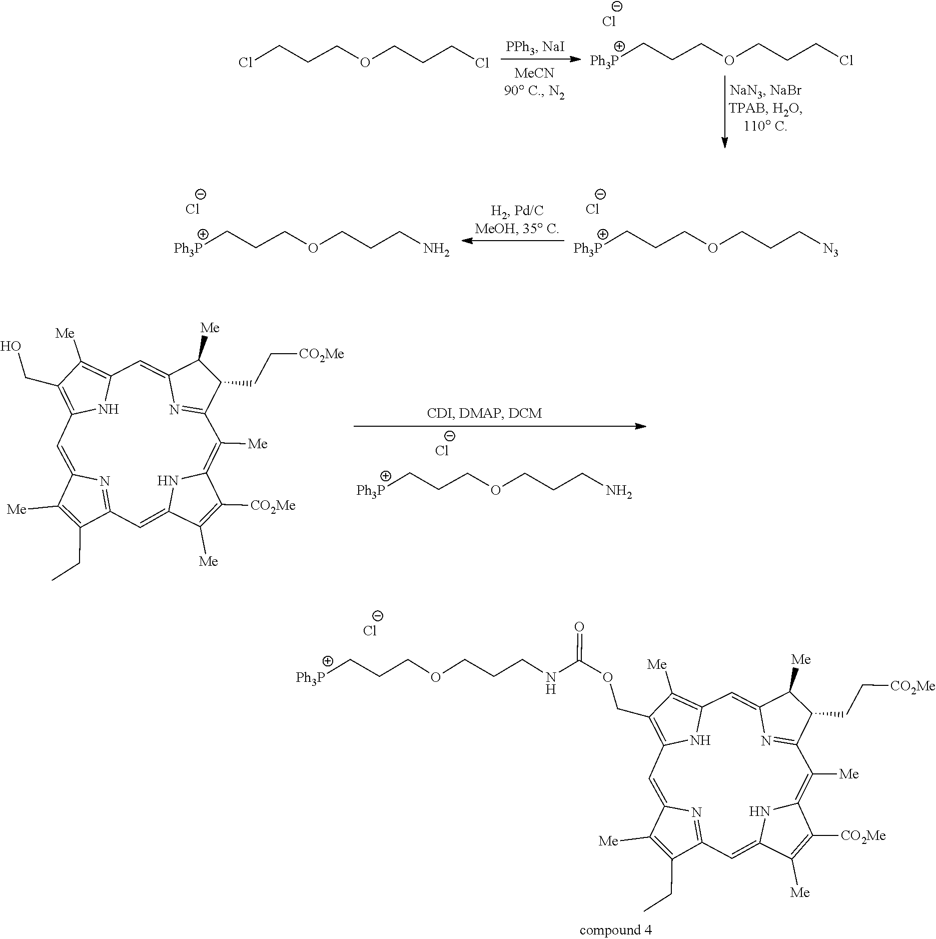

Step 1: A 500 mL 3-neck RBF, equipped with a 3 cm stirrer bar was charged with bis(3-chloropropyl)ether (20.00 g, 70.15 mmol, 1.66 eq), triphenyl phosphine (18.40 g, 70.15 mmol, 1 eq), sodium iodide (7.01 g, 46.77 mmol, 0.66 eq) and acetonitrile (340 mL). The RBF was set over an oil bath and fitted with an air condenser, where stirring (500 rpm) commenced under N₂ at an external temperature of 90° C. The mixture was left to stir for 72 hours. After this time, the reaction flask was cooled to room temperature and the suspension was filtered through a 2 cm plug of Celite®, washing through with acetonitrile (250 mL). The faint yellow solution was then evaporated to dryness to leave a dark yellow oil (44.40 g) which was subject to column chromatography (silica gel, 9×12 cm) using 6% MeOH in DCM as eluent. Fractions with R_{f}=0.35 as visualised by UV in 6% MeOH/DCM were combined and concentrated by rotary evaporation. The resulting residue was purified further by column chromatography (silica gel, 9×7 cm). DCM was used as eluent until no further triphenyl phosphine was present as observed by TLC, then the eluent was changed to 10% MeOH in DCM to remove the product from the column. Fractions with R_{f}=0.35 as visualised by UV in 6% MeOH/DCM were combined and concentrated by rotary evaporation to give (3-(3-chloropropoxy)propyl)triphenylphosphonium chloride as a faint red solid (18.74 g, 62%).

¹H NMR (400 MHz, CDCl₃) δ 7.87-7.76 (m, 9H), 7.75-7.63 (m, 6H), 3.93-3.80 (m, 2H), 3.75 (td, J=5-7, 1.3 Hz, 2H), 3.58 (q, J=6.3 Hz, 4H), 2.05-1.88 (m, 4H).

Step 2: To a 50 mL RBF was added (3-(3-chloropropoxy)propyl)triphenylphosphonium chloride (4.0 g, 9.23 mmol, 1 eq), NaN₃ (11.08 g, 1.2 eq), NaBr (38 mg, 0.04 eq), tetrapropylammonium bromide (49 mg, 0.02 eq) and water (10 mL). After connecting a water condenser, the flask was heated at 110° C. with stirring for 44 hours. Then the mixture was cooled and EtOAc (50 mL) was added. The mixture was transferred to a separating funnel and washed with water (3×30 mL) and brine (30 mL). The combined aqueous layers were extracted with DCM (3×20 mL). The combined organic layers were then dried (MgSO₄), filtered and concentrated to give (3-(3-azidopropoxy)propyl)triphenylphosphonium chloride as a pale yellow solid (3.20 g, 79%).

¹H NMR (400 MHz, CDCl₃) δ 7.86-7.77 (m, 9H), 7.73-7.66 (m, 6H), 3.90-3.80 (m, 2H), 3.75 (t, 2H), 3.53 (t, 2H), 3.33 (t, 2H), 1.99-1.89 (m, 2H), 1.82 (p, 2H).

Step 3: A 3-neck 100 mL RBF was charged with (3-(3-azidopropoxy)propyl)triphenylphosphonium chloride (1.00 g, 2.273 mmol, 1 eq), 10% Pd/C (20 mg), methanol (10 mL) and a stirrer bar. A hydrogen balloon was connected to the middle joint of the flask via a short length air condenser and the side-arm was connected to a 3-way tap. The setup was evacuated and then re-filled with nitrogen (3 times), evacuated and re-filled with hydrogen (2 times). The resulting solution was then stirred (550 rpm) under the hydrogen atmosphere for 2 hours at 35° C. Then the solution was filtered through Celite® (0.5×3 cm), washing with chloroform (2×10 mL) and the solvent then removed under reduced pressure to give (3-(3-aminopropoxy)propyl)triphenylphosphonium chloride as a viscous oil that solidified on standing (1.05 g, quantitative).

¹H NMR (400 MHz, CDCl₃) δ7.86-7.76 (m, 9H), 7.73-7.66 (m, 6H), 3.88-3.79 (m, 2H), 3.73 (t, 2H), 3.51 (t, 2H), 2.75 (t, 2H), 1.99-1.87 (m, 2H), 1.68 (p, 2H), 1.41 (brs, 2H).

Step 4: To a 50 mL RBF was added chlorin e4 13-hydroxymethyl dimethyl ester (100 mg, 0.171 mmol, 1 eq), carbonyl diimidazole (55 mg, 0.342 mmol, 2 eq), DCM (5 mL) and DMAP (10 mg). The resultant mixture was stirred under nitrogen for 3 hours with monitoring by TLC. (3-(3-Aminopropoxy)propyl)triphenylphosphonium chloride (354 mg, 0.855 mmol, 5 eq in DCM 2 mL) was added and stirring was continued for 20 hours. The reaction mixture was diluted with DCM (20 mL), transferred to a separatory funnel and washed with 1 M HCl (2×25 mL) and pH=7 phosphate buffer (25 mL) before being dried (Na₂SO₄) and concentrated by rotary evaporation to give a dark green residue. The residue was purified by column chromatography using 5% MeOH/DCM, loaded as a solution in the eluent. Fractions containing the major dark green spot (R_{f}=0.20 in 5% MeOH/DCM) were combined to give compound 4 (113 mg, 64%).

¹H NMR (400 MHz, CDCl₃) δ 9.66 (s, 1H), 9.55 (s, 1H), 8.74 (s, 1H), 7.66-7.51 (m, 6H), 7.51-7.36 (m, 10H), 6.35 (s, 2H), 5.46 (t, J=5.9 Hz, 1H), 4.50-4.40 (m, 2H), 4.30 (s, 3H), 3.81 (s, 3H), 3.77 (q, J=7.7 Hz, 2H), 3.71-3.62 (m, 4H), 3.60 (s, 3H), 3.58 (s, 3H), 3.51 (t, J=5.8 Hz, 1H), 3.45 (s, 3H), 3.38 (q, J=6.3 Hz, 2H), 3.27 (s, 3H), 2.59 (dt, J=15.8, 8.0 Hz, 1H), 2.42 (ddt, J=10.5, 7.8, 4.2 Hz, 1H), 2.28-2.16 (m, 1H), 1.90 (dtd, J=14.1, 9.0, 4.9 Hz, 1H), 1.81-1.64 (m, 11H), −1.63 (s, 1H), −1.86 (s, 1H).

### Synthesis Example 5—Synthesis of chlorin e4 dimethyl ester 13-(N-(3-triphenylphosphoniumpropyl)bromide)carbamate (compound 5)

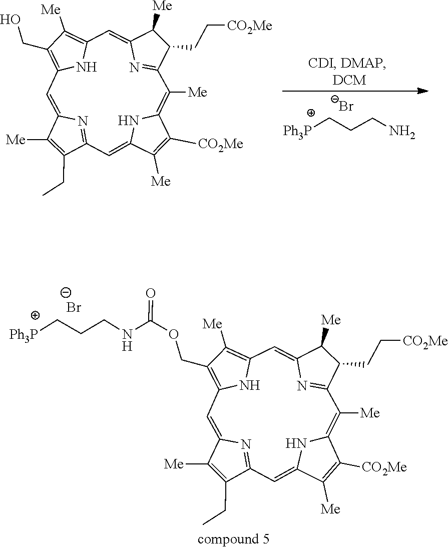

To a 25 mL RBF was added chlorin e4 13-hydroxymethyl dimethyl ester (120 mg, 0.205 mmol, 1 eq), carbonyl diimidazole (66 mg, 0.410 mmol, 2 eq), DCM (3 mL) and DMAP (5 mg). The resultant mixture was stirred under nitrogen for 3 hours with monitoring by TLC. A solution of (3-aminopropyl)triphenylphosphonium bromide (412 mg, 1.03 mmol, 5 eq) in DCM (5 mL) was added and stirring was continued for 16 hours. The reaction mixture was diluted with DCM (20 mL), transferred to a separatory funnel and washed with 1 M HCl (2×25 mL) and pH=7 phosphate buffer (25 mL) before being dried (Na₂SO₄) and concentrated by rotary evaporation to give a dark blue-green residue. The residue was purified by column chromatography using 5-10% MeOH/DCM, loaded as a solution in 5% MeOH/DCM. Fractions containing the major dark green spot (R_{f}=0.20 in 5% MeOH/DCM) were combined to give compound 5 as a dark blue solid (104 mg, 50%).

¹H NMR (400 MHz, CDCl₃) δ 9.75 (s, 1H), 9.67 (s, 1H), 8.77 (s, 1H), 7.80 (s, 1H), 7.50-7.38 (m, 6H), 7.23-7.10 (m, 10H), 6.95 (s, 1H), 6.69 (s, 1H), 6.39 (s, 2H), 4.46 (dq, J=14.5, 7.9, 7.2 Hz, 2H), 4.31 (s, 3H), 3.83 (s, 3H), 3.81-3.65 (m, 2H), 3.62 (s, 3H), 3.59 (s, 3H), 3.53 (s, 4H), 3.32 (s, 3H), 2.61 (dt, J=15.8, 8.0 Hz, 1H), 2.44 (q, J=8.6, 8.1 Hz, 1H), 2.25 (ddd, J=15.6, 8.5, 4.9 Hz, 1H), 1.97-1.85 (m, 1H), 1.80 (s, 3H), 1.75 (d, J=7.2 Hz, 2H), 1.68 (t, J=7.6 Hz, 4H), −1.63 (s, 1H), −1.76 (s, 1H).

### Synthesis Example 6—Synthesis of chlorin e4 dimethyl ester 13-(N-(2-triphenylphosphoniumethyl)bromide)carbamate (compound 6)

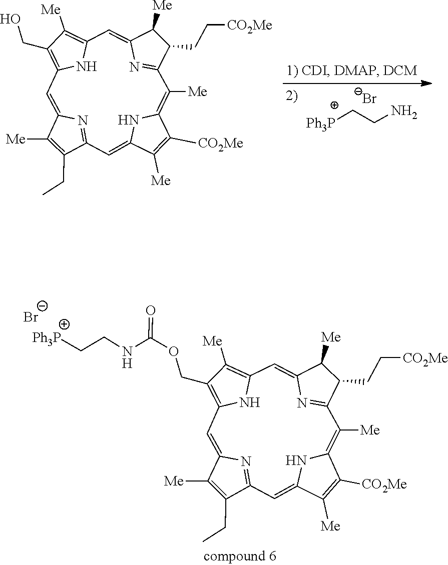

To a 25 mL RBF was added chlorin e4 13-hydroxymethyl dimethyl ester (120 mg, 0.205 mmol, 1 eq), carbonyl diimidazole (66 mg, 0.410 mmol, 2 eq), DCM (3 mL) and DMAP (5 mg). The resultant mixture was stirred under nitrogen for 3 hours and the reaction progress was monitored by TLC. A solution of 2-aminoethyltriphenylphosphonium bromide (398 mg, 1.03 mmol, 5 eq) in DCM (5 mL) was added and stirring was continued for 18 hours. The reaction mixture was diluted with DCM (20 mL), transferred to a separatory funnel and washed with 1 M HCl (2×25 mL) and pH=7 phosphate buffer (25 mL) before being dried (Na₂SO₄) and concentrated by rotary evaporation to give a dark blue-green residue. The residue was purified by column chromatography using 5% to 7.5% to 10% MeOH/DCM as eluent, loaded as a solution in 5% MeOH/DCM. Fractions containing the major dark green band (R_{f}=0.37 in 10% MeOH/DCM) were concentrated by rotary evaporation to give compound 6 as a dark blue solid (71 mg, 35%).

¹H NMR (400 MHz, CDCl₃) δ 9.66 (s, 1H), 9.61 (s, 1H), 8.76 (s, 1H), 7.73-7.57 (m, 7H), 7.44-7.35 (m, 8H), 6.25 (s, 2H), 4.53-4.40 (m, 2H), 4.29 (s, 3H), 3.89-3.70 (m, 7H), 3.60 (s, 3H), 3.58 (s, 3H), 3.47 (s, 3H), 3.32 (s, 3H), 2.59 (dt, J=15.8, 7.9 Hz, 1H), 2.51-2.37 (m, 1H), 2.23 (ddd, J=15.4, 8.3, 4.9 Hz, 1H), 1.92 (dtd, J=14.1, 9.0, 4.9 Hz, 1H), 1.83-1.64 (m, 8H), −1.64 (s, 1H), −1.82 (s, 1H).

### Synthesis Example 7—Synthesis of chlorin e4 13-(N-methyl-5-triphenylphosphonium bromide pentanamide) dimethyl ester (compound 7)

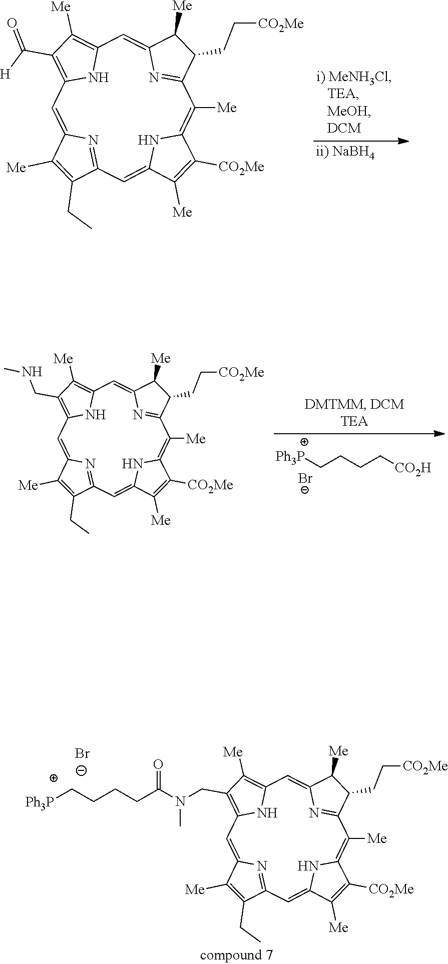

Step 1: To a 250 mL RBF was added chlorin e4 13-formyl dimethyl ester (1.0 g, 1 eq), DCM (20 mL), methanol (80 mL), TEA (289 mg, 3 eq) and methylamine hydrochloride (434 mg, 3 eq). The resultant mixture was stirred under nitrogen in the dark for 2 hours and then a further portion of methylamine hydrochloride (434 mg, 3 eq), TEA (289 mg, 3 eq) and 4 Å sieves (100 mg) were added and stirring continued for 4 hours. NaBH₄ (72 mg, 1.906 mmol, 10 eq) was added and stirring was continued for 12 hours. The reaction was acidified with 2 M HCl (10 mL) and stirred for 10 minutes. Phosphate buffer pH=7 (30 mL) was added and the mixture was extracted with DCM (3×50 mL). The combined organics were dried (Na₂SO₄) and concentrated by rotary evaporation to give a dark green residue which was subjected to silica gel column chromatography. The crude product was dissolved in DCM to load onto the column that had been pre-equilibrated with DCM. The column was eluted using a gradient of 3-7% MeOH/DCM. Fractions containing the product (major dark green spot, R_{f}=0.3 in 10% MeOH/DCM) were combined to give chlorin e4 13-N-methylamino dimethyl ester as a dark green solid (600 mg, 58%).

¹H NMR (400 MHz, CDCl₃) δ 9.51 (s, 1H), 9.42 (s, 1H), 8.68 (s, 1H), 4.82 (brm, 2H), 4.41 (m, 2H), 4.29 (s, 3H), 3.82 (s, 3H), 3.71 (q, 2H), 3.62-3.58 (m, 3H), 3.52 (s, 3H), 3.48 (s, 3H), 3.32 (s, 3H), 3.25 (s, 3H), 2.62 (s, 3H), 2.59-2.50 (m, 2H), 2.40-2.30 (m, 2H), 2.20-2.10 (m, 1H), 1.88-1.74 (m, 1H), 1.70-1.60 (m, 6H), −1.65 (brs, 1H), −1.90 (brs, 1H).

Step 2: To a 50 mL RBF was added chlorin e4 13-N-methylamino dimethyl ester (200 mg, 1 eq), (4-carboxybutyl)triphenylphosphonium bromide (296 mg, 2 eq), DCM (10 mL) and DMTMM (184 mg, 2 eq). The resultant mixture was stirred (250 rpm) under nitrogen at ambient temperature in the dark. After 10 minutes TEA (5 drops) was added and stirring continued for a further 2 hours. The reaction mixture was transferred to a separatory funnel, diluted with DCM (30 mL) and washed with water (10 mL). The aqueous layer was re-extracted with DCM (2×5 mL). The organic phase was dried (Na₂SO₄) and concentrated by rotary evaporation to give a blue-black film which was subjected to column chromatography. The crude product was dissolved in DCM and eluted using a gradient of 4-7% MeOH/DCM. Fractions containing the product (major dark green spot, R_{f}=0.3 in 7% MeOH/DCM) were combined and concentrated by rotary evaporation to give compound 7 (320 mg, 93%).

¹H NMR (400 MHz, CDCl₃) δ 9.71 (s, 1H), 9.62 (s, 1H), 8.70 (s, 1H), 7.75-7.67 (m, 6H), 7.55-7.45 (m, 9H), 5.85 (brm, 2H), 4.50-4.40 (m, 2H), 4.29 (s, 3H), 4.02-3.82 (m, 2H), 3.80-3.70 (m, 5H), 3.62 (s, 3H), 3.55 (s, 3H), 3.42 (s, 3H), 3.20 (s, 3H), 3.12 (s, 3H), 2.82 (t, 2H), 2.61-2.52 (m, 1H), 2.48-2.33 (m, 1H), 2.25-2.10 (m, 5H), 2.00-1.80 (m, 3H), 1.75-1.70 (m, 3H), 1.69-1.61 (m, 3H), −1.60 (brs, 1H), −1.80 (brs, 1H).

### Synthesis Example 8—Synthesis of chlorin e4 dimethyl ester 13-(N-methyl-(3-triphenylphosphoniumpropoxy)chloride) carbamate (compound 8)

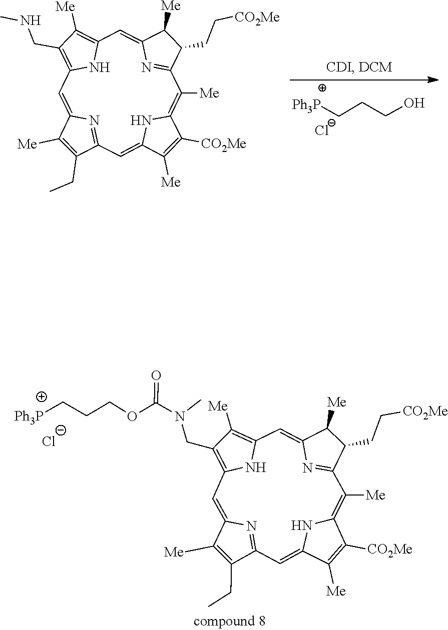

To a 25 mL RBF was added chlorin e4 13—N-methylamino dimethyl ester (100 mg, 0.167 mmol, 1 eq), carbonyl diimidazole (54 mg, 0.334 mmol, 1.5 eq) and DCM (4 mL). The resultant mixture was stirred (300 rpm) under nitrogen for 1 hour. (3-Hydroxypropyl)triphenylphosphonium chloride (119 mg, 0.334 mmol, 1.5 eq) in DCM (2 mL) was added and stirring was continued overnight in the dark at 23° C. Then the reaction mixture was diluted with DCM (15 mL), transferred to a separatory funnel and washed with water (15 mL), extracted with DCM (2×5 mL), dried (Na₂SO₄) and concentrated by rotary evaporation to give a dark green residue. The residue was subjected to column chromatography. The residue was dissolved in DCM to load onto the column which had been pre-equilibrated with DCM. The column was eluted using a gradient of 3-8% MeOH/DCM. Fractions containing the product (major dark green spot, R_{f}=0.25 in 7% MeOH/DCM) were combined to give compound 8 as a dark green solid (72 mg, 44%).

¹H NMR (400 MHz, CDCl₃) δ 9.73 (s, 1H), 9.55 (s, 1H), 8.72 (m, 1H), 7.75-7.60 (m, 6H), 7.42-7.30 (m, 9H), 5.82-6.68 (m, 2H), 4.62 (m, 1H), 4.41 (m, 2H), 3.85-3.70 (m, 5H), 3.60 (m, 6H), 3.45-3.35 (m, 3H), 3.20 (s, 3H), 2.62-2.46 (m, 2H), 2.25-2.00 (m, 4H), 1.92-1.84 (m, 4H), 1.80-1.65 (m, 8H), −1.55 (brs, 1H), −1.75 (brs, 1H).

### Biological Experimental Details

### Example 1—Determination of Solubility of Chlorin e4 Analogues

Absorbance maxima were used as a surrogate measure of solubility. The relevant chlorin e4 analogue was diluted to 50 μM in PBS (phosphate buffered saline) solutions containing decreasing amounts of DMSO from 100% to 0%.

Where required, polyvinylpyrrolidone (K30) was added to a final concentration of 1% w/v. Absorbance was measured using a Cytation 3 Multimode Plate Reader (Biotek) in spectral scanning mode, with spectra captured between 500-800 nm in 2 nm increments. Equivalent blank solutions were also measured and subtracted accordingly. Each spectrum was normalized to have a minimum signal of 0, and a maximum signal in pure DMSO solution (the most soluble state) of 100%.

### Example 2—Cytotoxicity, Phototoxicity and Therapeutic Index

### Preparation of Photosensitizer Stock Solutions

Photosensitizers (e.g. chlorin e4 analogue, chlorin e4 disodium (provided by Advanced Molecular Technologies, Scoresby) or Talaporfin sodium (purchased from Focus Bioscience cat #HY-16477-5MG)) were resuspended in 100% dimethylsulfoxide (DMSO) at a concentration of 5.5 mM. Samples were stored at 4° C. protected from light.

### Preparation of Photosensitizers for In Vitro Studies

For in vitro experiments, photosensitizers (stock solution 5.5 mM in 100% DMSO) were diluted 1:100 in concentrated excipient solution (final 55 μM photosensitizer in 10% w/v Kollidon-12, 42.4% w/v polysorbate 80, 0.6% w/v citric acid anhydrous, 40% w/v ethanol, 1.0% DMSO). Serial dilutions were prepared in cell culture media (Dulbecco's Modified Eagle Medium/Nutrient Mixture F-12 (DMEM/F-12)) supplemented with 10% v/v Fetal Bovine Serum, 100 U/mL penicillin, 100 μg/mL streptomycin and the same excipient solution at a constant 1:55 dilution.

### Cell Culture

Human ovarian cancer cell line SKOV3 (ATCC #HTB-77) was maintained in Dulbecco's Modified Eagle Medium/Nutrient Mixture F-12 (DMEM/F-12), supplemented with 10% v/v Fetal Bovine Serum, 100 U/mL penicillin and 100 μg/mL streptomycin. Monolayer cultures were grown in a humidified incubator at 37° C. with 5% CO₂. Once cells had reached ˜80% confluence, spent media was replaced with media containing photosensitizer at the required concentration and cells were incubated for the desired period of time to allow photosensitizer uptake.

### Statistical Analyses

All data were analysed using GraphPad PRISM v8.3.1 (549) (GraphPad Software, CA). Spectral absorbance and viability measurements were normalized in the range 0-100%, with a minimum of 0 and a maximum value determined from the dataset. Dose response was determined using a sigmoidal four-point non-linear regression with variable slope, and IC10 or IC90 calculated for each compound. All data are shown as mean±SD (where appropriate).

### Cytotoxicity

SKOV3 cells were seeded in 96-well black wall plates (Greiner #655090) at a cell density of 5000 cells in 100 μl culture medium per well. On reaching ˜60% confluence, media was aspirated and replaced with fresh media containing the relevant chlorin e4 analogue from 0-100 μM in DMSO. Cells were incubated for a further 24 hours, allowing uptake of chlorin e4 analogues.

To test for inherent cytotoxicity (i.e. “dark toxicity”) of the chlorin e4 analogues, the culture media was replaced after 24 hours with fresh media containing 10% (v/v) AlamarBlue Cell Viability Reagent (ThermoFisher) and cells incubated at 37° C. for 6 hours. Untreated cells were used as a control. Fluorescence (Ex 555 nm/Em 596 nm) was measured using a Cytation 3 Cell Imaging Multi-Mode Reader (Biotek), and cytotoxicity assessed according to the % viable cells remaining. All measurements were made in quadruplicate.

### Phototoxicity

SKOV3 cells were seeded in 96-well black wall plates (Greiner #655090) at a cell density of 5000 cells in 100 μl culture medium per well. On reaching ˜60% confluence, media was aspirated and replaced with fresh media containing the relevant chlorin e4 analogue from 0-100 μM in DMSO. Cells were incubated for a further 24 hours, allowing uptake of chlorin e4 analogues.

To test for phototoxicity, cells incubated with chlorin e4 analogues (0-10 μM in DMSO) had culture media replaced after 24 hours (as above) and were then exposed to a 660 nm laser (Invion) or light-emitting diode (LED) panel (Invion) with optical power density at 50 mW/cm² for 5 mins (total 15 J/cm²). Laser and LED exposure induce an equivalent response in relation to phototoxicity. Following activation, cells were cultured for a further 24 hours. Media was then replaced with fresh media containing AlamarBlue, and % viable cells remaining assessed as above. Controls included cells treated with chlorin e4 analogues but not activated by laser light; cells without chlorin e4 analogue treatment but with laser light; and untreated controls. All measurements were made in quadruplicate.

### Toxicity Profile for Chlorin e4 Analogues

The phototoxicity and inherent cytotoxicity (i.e. “dark toxicity”) of chlorin e4 analogues were assessed as previously using SKOV3 ovarian cancer cells. For comparative purposes, chlorin e4 analogues were compared against chlorin e4 disodium and Talaporfin sodium, a clinically approved photosensitizer used in the photodynamic treatment of lung cancers. Phototoxicity IC90 values and dark toxicity IC10 values were calculated using a log[inhibitor]-vs normalized response dose curve with variable slope, using the formula Y=100/(1+(IC90/X)\hat{ }HillSlope (phototoxicity IC90)) or Y=100/(1+(IC10/X)\hat{ }HillSlope (dark toxicity IC10)).

Phototoxicity and dark toxicity values are provided in Table 1. All chlorin e4 analogues had phototoxicity IC90 values in the nM range; with an IC90 for four compounds below 5 nM (Table 1). These were substantially better than chlorin e4 disodium (IC90 21.32 μM) or Talaporfin sodium (IC90 22.83 μM); indeed, the best-performing compound (compound 6) achieved 4 orders of magnitude greater phototoxicity compared to Talaporfin sodium. Thus, chlorin e4 analogues achieved an up to ˜13,000-fold increase in phototoxicity compared to Talaporfin sodium, a clinically approved photosensitizer.

Substantial variation in the dark toxicity of the chlorin e4 analogues of the present invention was observed (Table 1). The greater phototoxicity afforded by the chlorin e4 analogues of the present invention, however, is expected to offset any dark toxicity issue through a decreased dose requirement in use.

### Therapeutic Index for Chlorin e4 Analogues

To evaluate the therapeutic potential of chlorin e4 analogues, the therapeutic index (TI) was calculated. TI provides a quantitative measurement to describe relative drug safety, by comparing the drug concentration required for desirable effects versus the concentration resulting in undesirable off-target toxicity. TI was calculated using phototoxicity IC90 vs dark toxicity IC10.

TI values are provided in Table 1. Talaporfin sodium had a low therapeutic index (TI=0.49) with chlorin e4 disodium only marginally better (TI=1.89), indicating that whilst their relative cytotoxicity is low, the potential therapeutic window for their use is small. The chlorin e4 analogues of the present invention had comparatively significantly improved TIs with substantially greater phototoxicity (Table 1).

Thus, the chlorin e4 analogues of the present invention have a desirable therapeutic index that is better than a clinically applied photosensitizer. Moreover the greater phototoxicity of the chlorin e4 analogues suggests their potential use at a greatly reduced dose in vivo. The chlorin e4 analogues therefore have an acceptable therapeutic profile for clinical application.

TABLE 1
Toxicity profile and therapeutic index for chlorin e4 analogues:

| | Phototoxicity | Dark toxicity | Therapeutic |
|---|---|---|---|
| Species | μM (IC90) | μM (IC10) | Index |
| Compound 1 | 0.06 | 8.64 | 144.00 |
| Compound 3 | 0.0041 * | 0.838 | 204.39 |
| Compound 4 | 0.0024 * | 3.881 | 1,617.08 |
| Compound 5 | 0.0029 * | 3.046 | 1,050.34 |
| Compound 6 | 0.0018 * | 2.281 | 1,267.22 |
| Chlorin e4 13-hydroxymethyl | 0.3248 * | 42.830 | 131.87 |
| dimethyl ester | | | |
| Chlorin e4 disodium | 21.32 | 40.23 | 1.89 |
| Talaporfin sodium | 22.83 | 11.10 | 0.49 |

* denotes that the phototoxicity was measured by LED

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I) or a complex of formula (II): 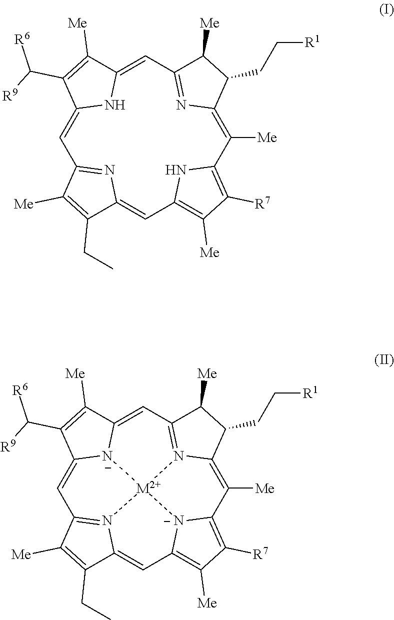
or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]y, —R^{α}—[P(R⁵)₃]Y, —R^{α}—[R⁸]y, —R^{α}—[N(R⁵)₂(R^{5′})], —R^{α}—[P(R⁵)₂(R^{5′})] or —R^{α}—[R^{8′}];
—R^{α}—, each independently, is selected from a C₁-C₄₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂⁻, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X;
—R⁷ is selected from —C(O)—OR³, —C(O)—SR³, —C(O)—N(R³)₂, —C(S)—OR³, —C(S)—SR³ or —C(S)—N(R³)₂;
—R⁸ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂⁻ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is hydrogen or methyl;
n is 1, 2, 3, 4, 5 or 6;
X is a halo group;
Y is a counter anion;
Z is a counter cation; and
M²⁺ is a metal cation.

2. The compound or complex according to claim 1, wherein each —R^{α}— is independently selected from C₁-C₆ alkylene.

3. The compound or complex according to any preceding claim, wherein at least one of —R², —R³ and —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group.

4. The compound or complex according to claim 3, wherein —R^{β} is a saccharidyl group selected from:

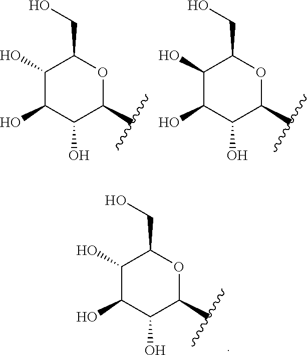

5. The compound or complex according to claim 4, wherein the saccharidyl group is:

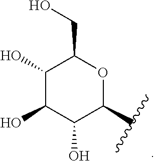 [SMILES: *[C@@H]1O[C@H](CO)[C@@H](O)[C@H](O)[C@H]1O]

6. The compound or complex according to claim 3, wherein —R^{β} is a saccharidyl group selected from: 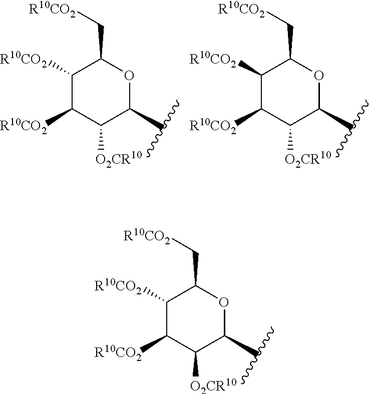
wherein —R¹⁰ is selected from C₁-C₄ alkyl.

7. The compound or complex according to claim 6, wherein —R¹⁰ is methyl.

8. The compound or complex according to any preceding claim, wherein —R¹ is —C(O)—OR³, R³ is —R^{β}, and —R^{β} is a C₁-C₄ alkyl group.

9. The compound or complex according to any one of claims 1-7, wherein —R¹ is selected from —C(O)—OR³, —C(O)—SR³ or —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl.

10. The compound or complex according to any preceding claim, wherein —R⁶ is selected from —OR² or —SR², and —R² is selected from —R^{α}—OR^{β}, —R^{α}—SR^{β}, —R^{α}—S(O)R^{β} or —R^{α}—S(O)₂R^{β}, and —R^{β} is a saccharidyl group.

11. The compound or complex according to any preceding claim, wherein —R⁷ is —C(O)—OR³ and —R³ is C₁-C₄ alkyl.

12. The compound or complex according to any one of claims 1-10, wherein —R⁷ is selected from —C(O)—OR³, —C(O)—SR³ or —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl.

13. The compound or complex according to claim 1, wherein the compound or complex is: 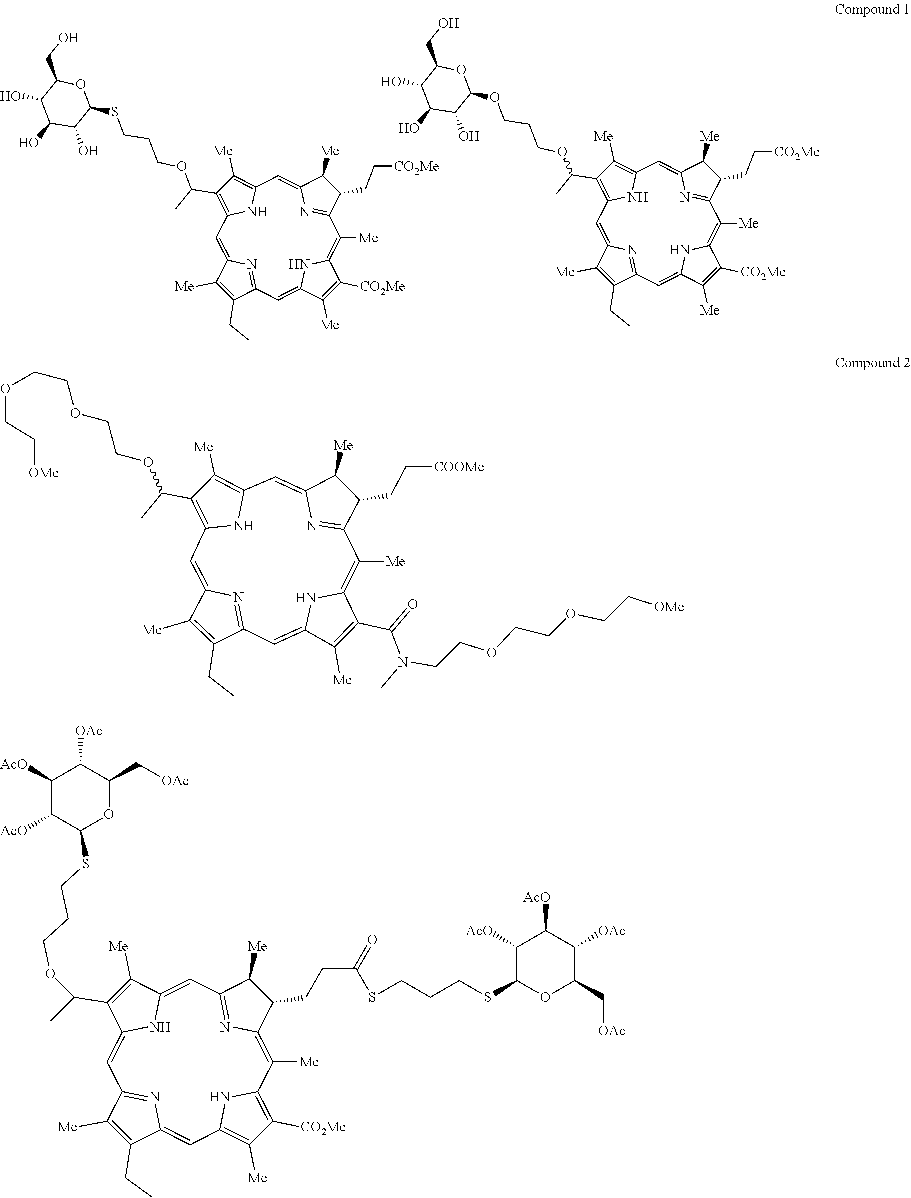 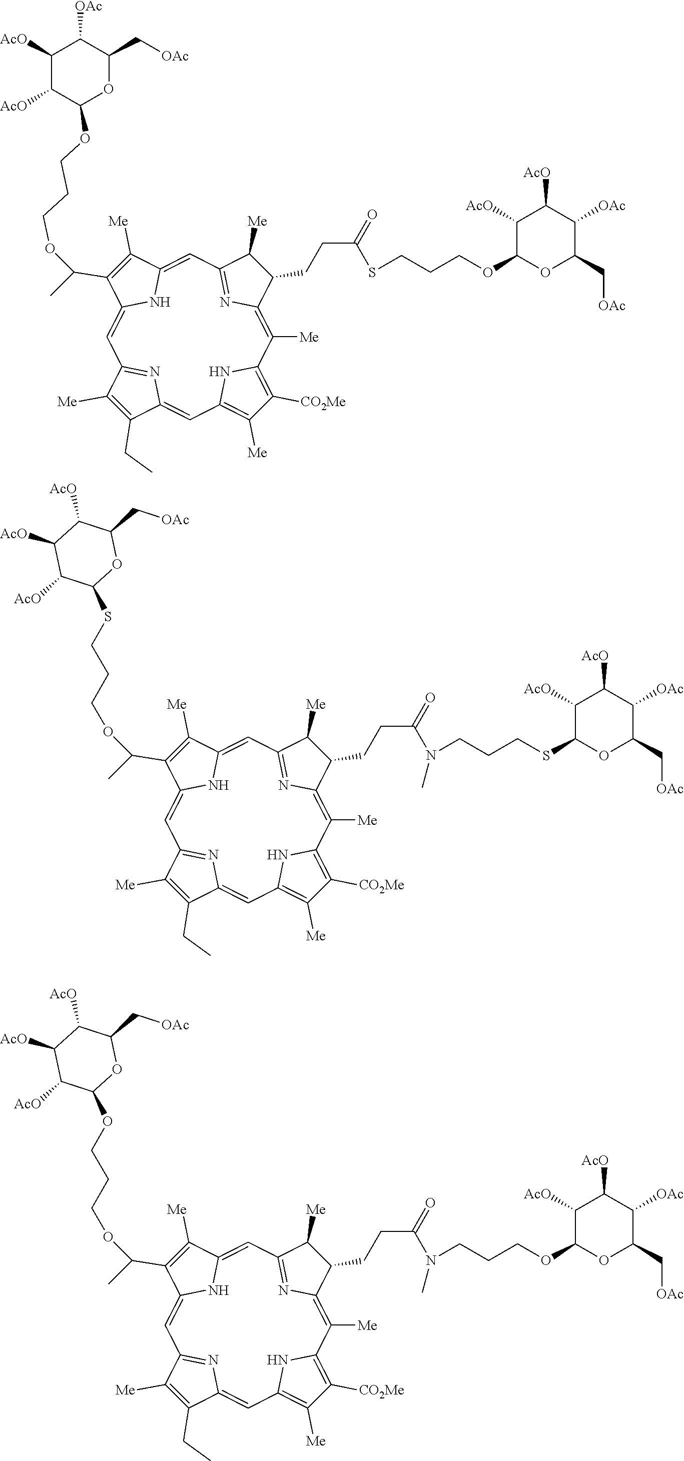 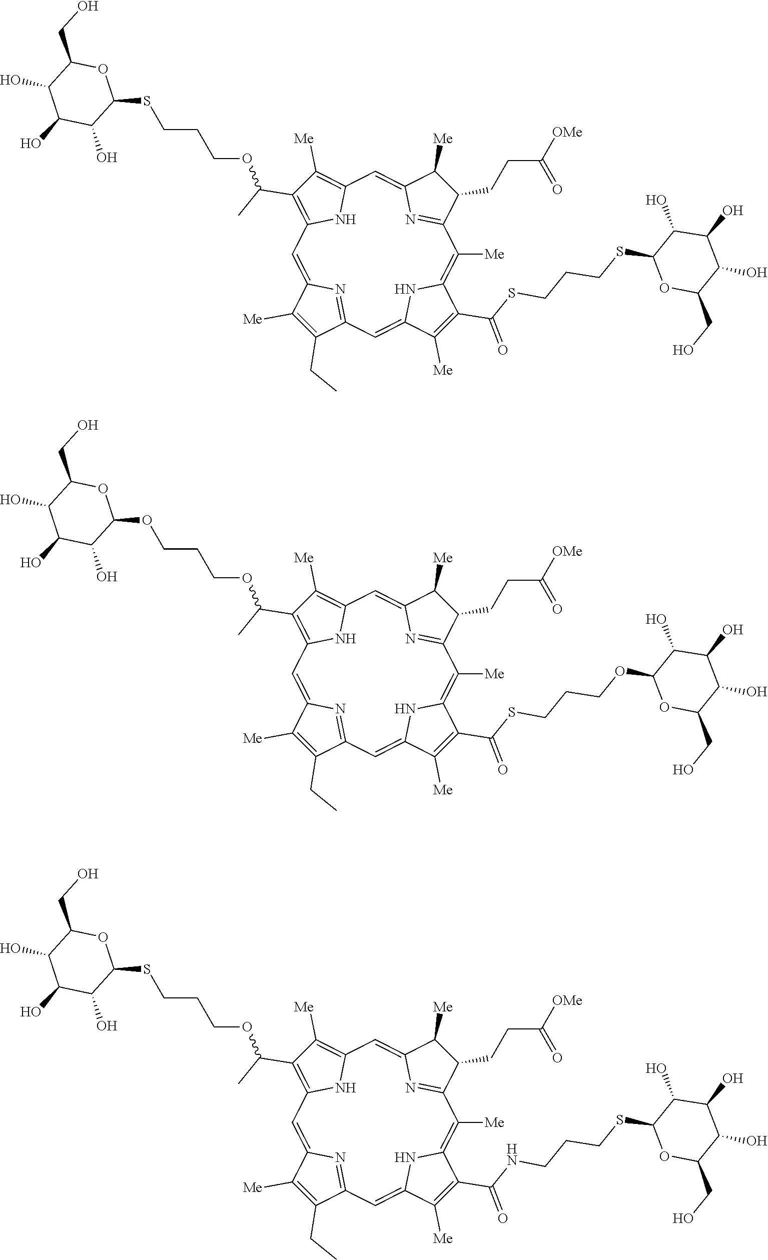 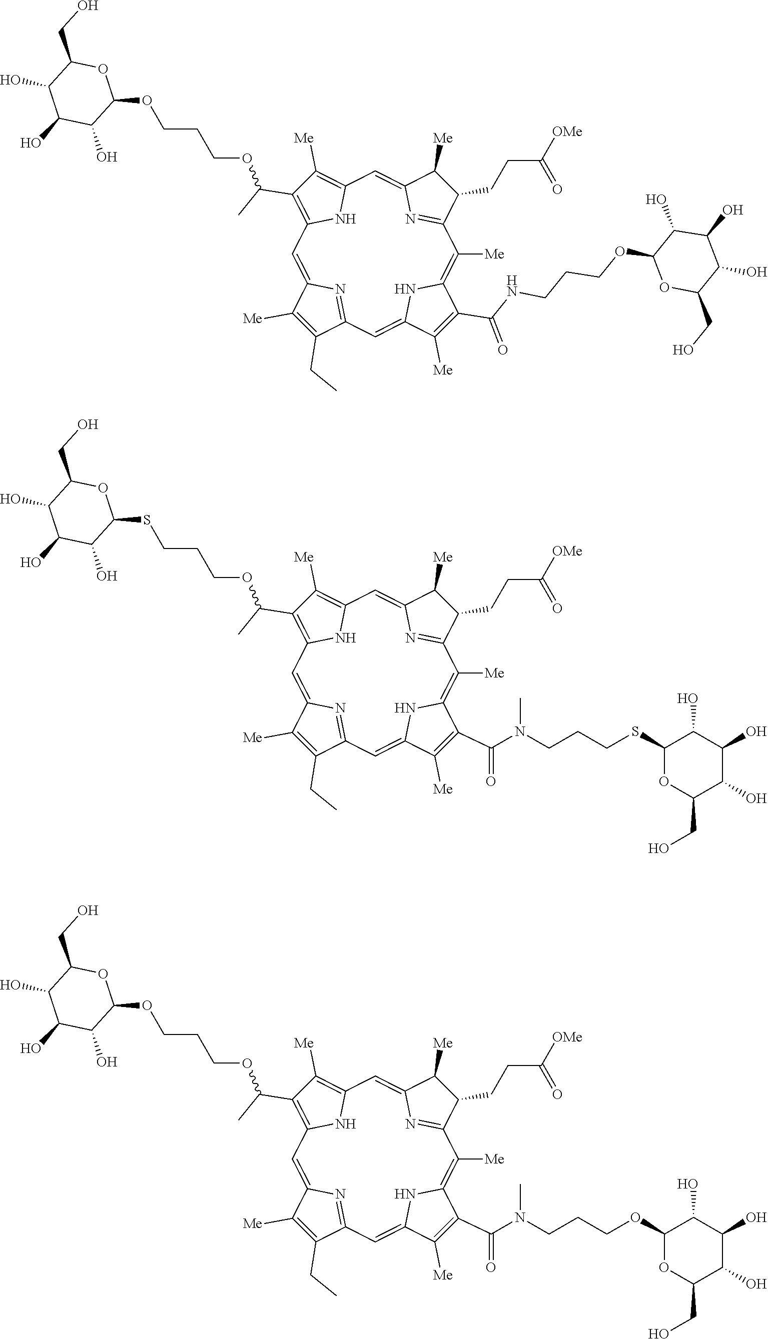 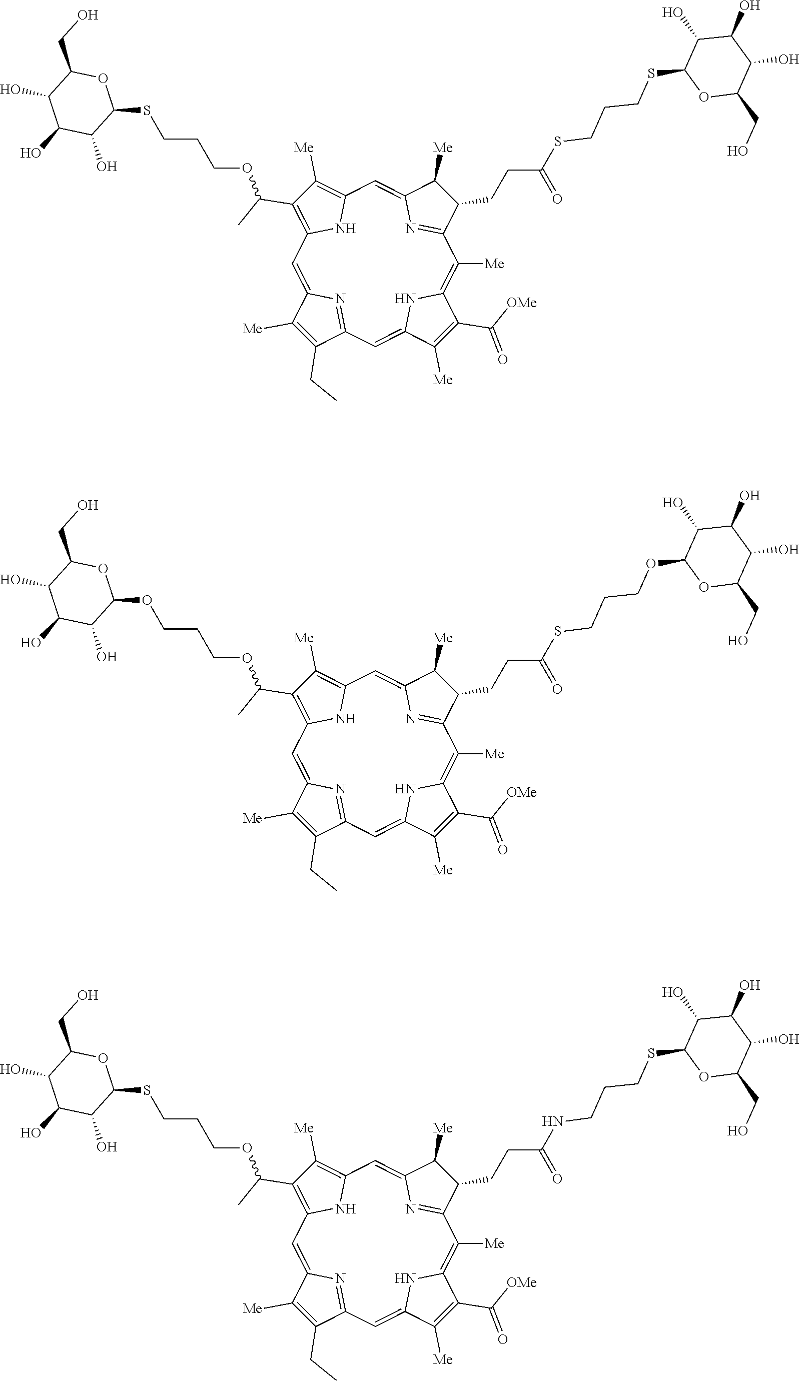 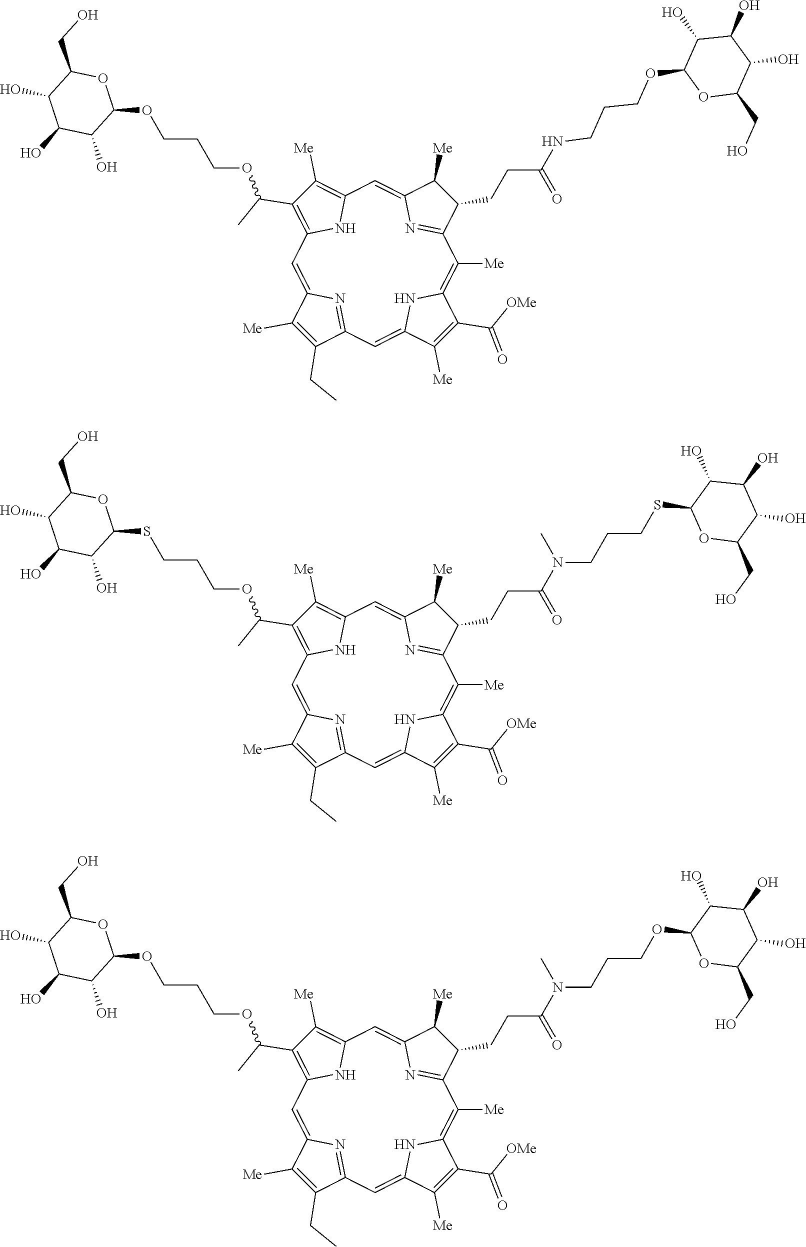 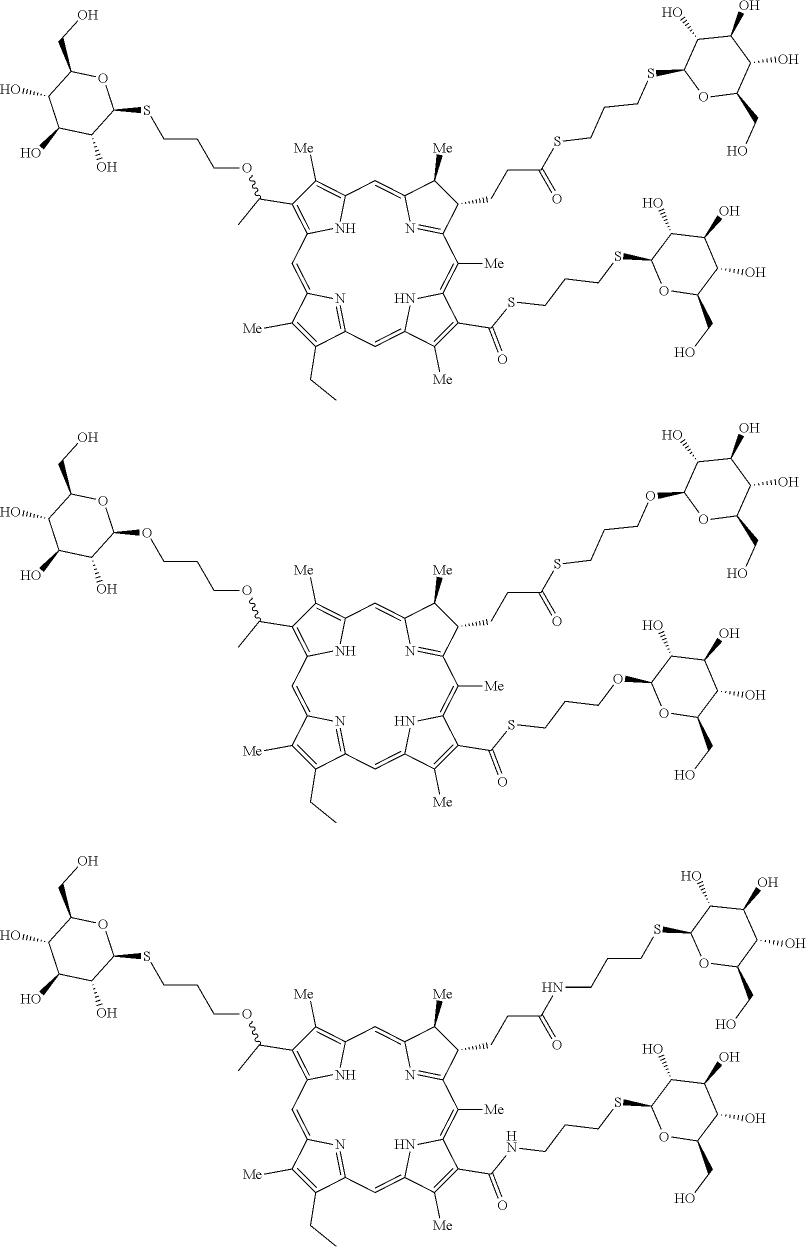 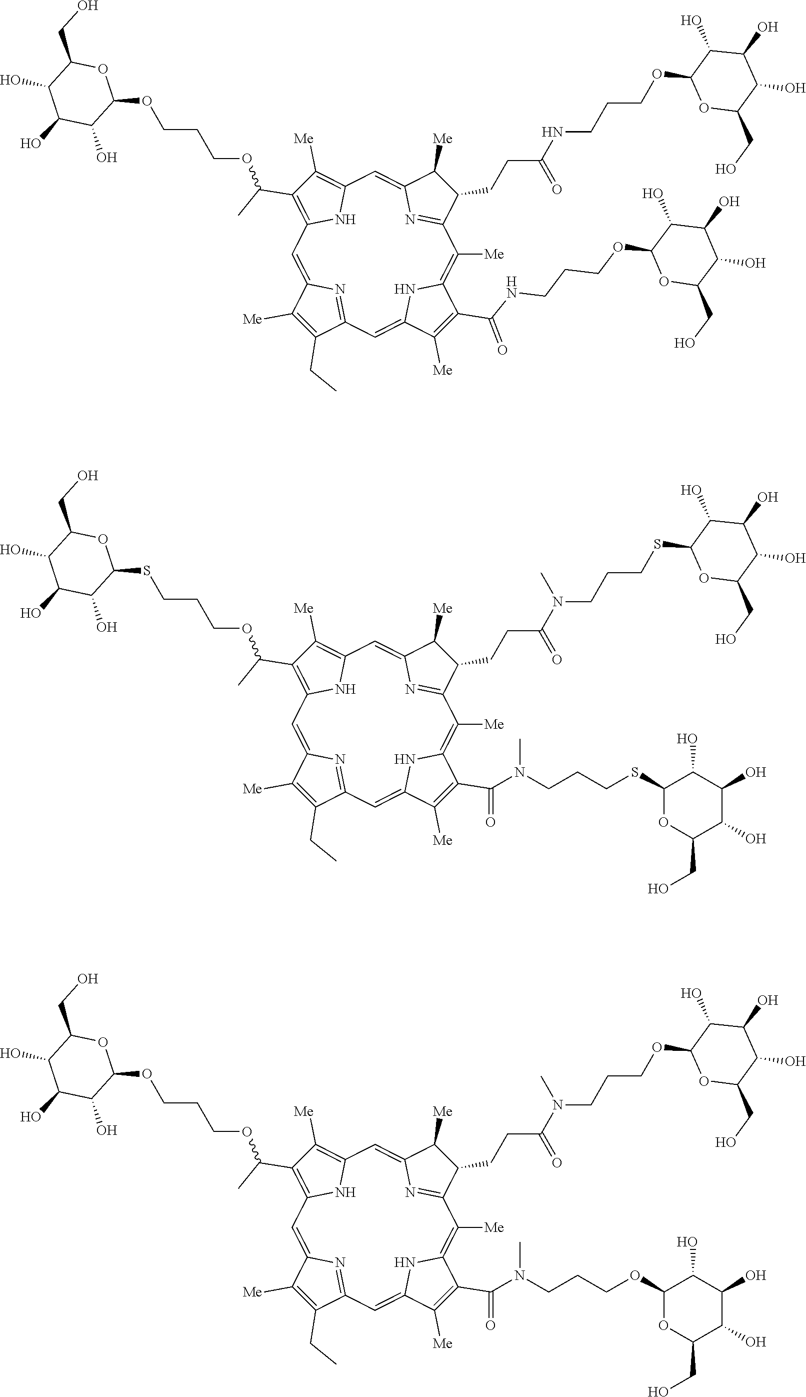 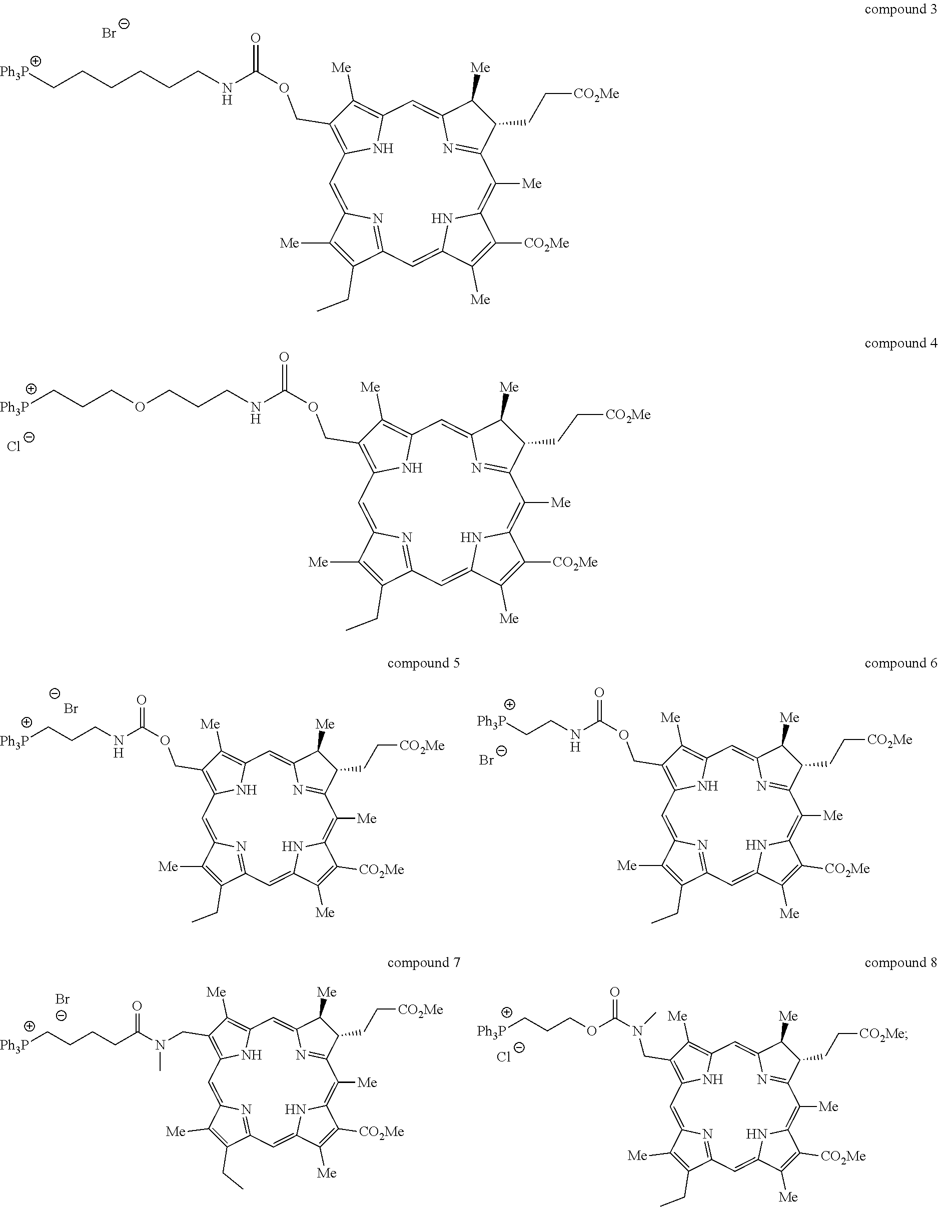
or a metal cation complex thereof, or a pharmaceutically acceptable salt thereof.

14. The compound or complex according to any preceding claim, for use in medicine.

15. The compound or complex according to any preceding claim, for use in photodynamic therapy or cytoluminescent therapy.

16. The compound or complex according to any preceding claim, for use in the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

17. The compound or complex according to any preceding claim, for use in the treatment of a disease characterised by benign or malignant cellular hyperproliferation or by areas of neovascularisation.

18. The compound or complex according to any preceding claim, for use in the treatment of a benign or malignant tumour.

19. The compound or complex according to any preceding claim, for use in the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

20. The compound or complex according to any preceding claim, for use in photodynamic diagnosis.

21. The compound or complex according to any preceding claim, wherein the compound is adapted for administration prior to administration of irradiation.

22. The compound or complex according to claim 21, wherein the irradiation is electromagnetic radiation with a wavelength in the range of from 500 nm to 1000 nm.

23. A pharmaceutical composition comprising a compound or complex according to any preceding claim and a pharmaceutically acceptable carrier or diluent.

24. The pharmaceutical composition according to claim 23, further comprising polyvinylpyrrolidone.

25. The pharmaceutical composition according to claim 23 or 24, further comprising an immune checkpoint inhibitor.

26. The pharmaceutical composition according to claim 25, wherein the immune checkpoint inhibitor is selected from Pembrolizumab, Nivolumab, Cemiplimab, Atezolizumab, Avelumab, Durvalumab or Ipilimumab.

27. The pharmaceutical composition according to any one of claims 23-26, for use in photodynamic therapy or cytoluminescent therapy.

28. The pharmaceutical composition according to any one of claims 23-27, for use in the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

29. The pharmaceutical composition according to any one of claims 23-28, for use in the treatment of a disease characterised by benign or malignant cellular hyperproliferation or by areas of neovascularisation.

30. The pharmaceutical composition according to any one of claims 23-29, for use in the treatment of a benign or malignant tumour.

31. The pharmaceutical composition according to any one of claims 23-30, for use in the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

32. The pharmaceutical composition according to claim 23 or 24, for use in photodynamic diagnosis.

33. The pharmaceutical composition according to any one of claims 23-32, wherein the pharmaceutical composition is adapted for administration prior to administration of irradiation.

34. The pharmaceutical composition according to claim 33, wherein the irradiation is electromagnetic radiation with a wavelength in the range of from 500 nm to 1000 nm.

35. The pharmaceutical composition according to any one of claims 23-34, wherein the pharmaceutical composition is in a form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration.

36. The pharmaceutical composition according to claim 35, wherein the pharmaceutical composition is in a form suitable for oral or parenteral administration.

37. Use of a compound or complex according to any one of claims 1-22, in the manufacture of a medicament for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

38. Use of a compound or complex according to any one of claims 1-22, in the manufacture of a phototherapeutic agent for use in photodynamic therapy or cytoluminescent therapy.

39. The use according to claim 38, wherein the phototherapeutic agent is for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

40. The use according to any one of claims 37-39, wherein the medicament or the phototherapeutic agent is for the treatment of a disease characterised by benign or malignant cellular hyperproliferation or by areas of neovascularisation.

41. The use according to any one of claims 37-40, wherein the medicament or the phototherapeutic agent is for the treatment of a benign or malignant tumour.

42. The use according to any one of claims 37-41, wherein the medicament or the phototherapeutic agent is for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

43. Use of a compound or complex according to any one of claims 1-22, in the manufacture of a photodiagnostic agent for use in photodynamic diagnosis.

44. The use according to any one of claims 37-43, wherein the medicament, the phototherapeutic agent or the photodiagnostic agent is adapted for administration prior to administration of irradiation.

45. The use according to claim 44, wherein the irradiation is electromagnetic radiation with a wavelength in the range of from 500 nm to 1000 nm.

46. A method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of a compound or complex according to any one of claims 1-22 to a human or animal in need thereof.

47. A method of photodynamic therapy or cytoluminescent therapy of a human or animal disease, the method comprising administering a therapeutically effective amount of a compound or complex according to any one of claims 1-22 to a human or animal in need thereof.

48. The method according to claim 47, wherein the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

49. The method according to any one of claims 46-48, wherein the human or animal disease is characterised by benign or malignant cellular hyperproliferation or by areas of neovascularisation.

50. The method according to any one of claims 46-49, wherein the human or animal disease is a benign or malignant tumour.

51. The method according to any one of claims 46-50, wherein the human or animal disease is early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, so larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

52. A method of photodynamic diagnosis of a human or animal disease, the method comprising administering a diagnostically effective amount of a compound or complex according to any one of claims 1-22 to a human or animal.

53. The method according to any one of claims 46-52, wherein the human or animal is subjected to irradiation after the administration of the compound or complex according to any one of claims 1-22.

54. The method according to claim 53, wherein the irradiation is electromagnetic radiation with a wavelength in the range of from 500 nm to 1000 nm.

55. A pharmaceutical combination or kit comprising:
(a) a compound or complex according to any one of claims 1-22; and
(b) a co-agent which is an immune checkpoint inhibitor.

56. The pharmaceutical combination or kit according to claim 55, wherein the immune checkpoint inhibitor is selected from Pembrolizumab, Nivolumab, Cemiplimab, Atezolizumab, Avelumab, Durvalumab or Ipilimumab.
